(19) United States
(12) Patent Application Publication
(10) Pub. No.: US 2018/0051340 A1
(43) Pub. Date: Feb. 22, 2018

(54) METHODS AND USES FOR DIAGNOSIS AND TREATMENT OF PROSTATE CANCER

(71) Applicant: BRITISH COLUMBIA CANCER AGENCY BRANCH, Vancouver (CA)

(72) Inventors: Cheryl D. HELGASON, Vancouver (CA); Francesco CREA, Vancouver (CA); Yuzhuo WANG, Vancouver (CA); Kim N. CHI, Vancouver (CA); Akira WATAHIKI, Nishinomiya (JP); Hui Hsuan LIU, Vancouver (CA); Abhijit PAROLIA, Allahabad U.P. (IN)

(21) Appl. No.: 14/901,618

(22) PCT Filed: Jun. 27, 2014
(86) PCT No.: PCT/CA2014/000538

§ 371 Date: Dec. 28, 2015

Related U.S. Application Data
(60) Provisional application No. 61/841,225, filed on Jun. 28, 2013.

(51) Int. Cl. C12Q 1/68; C12N 15/113; A61K 31/713

(52) U.S. Cl. CPC C12Q 1/6886; C12N 15/113; C12Q 2600/158; C12N 2310/14; A61K 31/713

(57) ABSTRACT

In an aspect, the invention provides methods and uses of PCAT18 for diagnosing, prognosing, and treatment monitoring of prostate cancer (PCa) in a subject. In another aspect, methods of treating PCa in a subject by administering an inhibiting agent of PCAT18 are provided. Uses of PCAT18 in treating PCa in a subject, and pharmaceutical compositions comprising a therapeutic agent effective to reduce the amount of PCAT18 in cancerous prostate cells and a pharmaceutically acceptable carrier, are also provided. The transcript of PCAT18 is a long non-coding RNA (lncRNA), whose expression is significantly altered in biological samples obtained from subjects with PCa or at risk of developing PCa compared to normal individuals. Expression of PCAT18 is specific to prostate tissue and is elevated in both cancerous prostate tissue and blood plasma of subjects with PCa relative to subjects without PCa or patients with other forms of cancer.

### FIELD OF THE INVENTION

The present invention relates to a novel biomarker for prostate cancer. In particular, the present invention relates to methods and uses of a novel long non-coding RNA (lncRNA), termed PCAT18, for the early detection, diagnosis, prognosis, classification, treatment monitoring, or treatment of prostate cancer (PCa).

### BACKGROUND OF THE INVENTION

The vast majority of prostate cancer (PCa)-related deaths are attributed to the progression from localized, indolent disease to metastatic castration-resistant PCa (mCRPC) (1). Despite enormous research efforts, risk stratification of PCa patients at diagnosis is still based on T stage, Gleason grade and plasma PSA levels, a method that overlooks many potentially aggressive cases (2) and can have false positives. For example, testing plasma PSA levels has a high false positive rate with only approximately 25% of men with elevated PSA levels actually having PCa. More importantly, rising PSA levels are not an accurate early indicator of disease progression. According to a recent meta-analysis, PSA screening does find additional cases of prostate cancer, but most studies do not show a corresponding effect on PCa-specific mortality. Of the patients with PCa, only a limited number will have disease progression or will die from their disease while a substantial proportion of men with clinically insignificant disease are being over treated. In other words, their disease will never cause morbidity or mortality. The use of the PSA test, therefore, is controversial. For example, the US Preventative Services Task Force does not recommend PSA screening and PSA screening is not routinely provided in Canada.

Human transcriptome analysis has recently revealed that most RNA molecules produced in human cells are not translated, and thus protein-coding genes account for only a small percentage of all RNAs (3). These non-coding transcripts include the well-so known ribosomal-, transfer- and micro-RNAs (rRNA, tRNA, miRNA respectively). MiRNA profiling in tumor specimens and patient-derived biological fluids is emerging as a powerful tool to differentiate localized and metastatic PCa (4). A less investigated class of non-coding RNAs is represented by long non-coding RNAs (lncRNAs), i.e. transcripts longer than 200 bp with no protein-coding function (5). Recent evidence indicates that lncRNAs may be an overlooked source of cancer biomarkers and therapeutic targets. The term lncRNA has been used as a catch-all definition, including poly-adenylated and non-poly-adenylated sequences, as well as intergenic and intronic transcripts. Estimates suggest the number of human lncRNAs rivals the count of protein-coding genes, ranging from 10,000 to 20,000 (6). Despite these large numbers, only a handful of lncRNAs have been characterized. Notably, most characterized lncRNAs display deregulated expression in cancer cells, where they play oncogenic or tumor suppressive functions (6). A striking feature of some lncRNAs is their tissue-specificity which prompted some authors to propose them as novel biomarkers (6). Two previously characterized lncRNAs (PCGEM1 and PCA3) are specifically expressed in PCa compared to an array of normal and neoplastic tissues (7, 8). PCA3 is present in urine samples from PCa patients and is able to detect the disease with 77.5% sensitivity and 57.1% specificity (9). PCA3 levels, however, are not able to discriminate between indolent and clinically aggressive PCa (9). The clinical utility of PCGEM1 has also not been determined. Accordingly, it is unclear whether PCA3 or PCGEM1 is a viable therapeutic target.

A new diagnostic, prognostic and therapeutic biomarker is, therefore, needed for early recognition, detection, diagnosis and effective management of PCa. In particular, such a biomarker should be able to distinguish between localized, indolent PCa and clinically aggressive PCa and detectable in a subject's blood, urine, saliva, plasma or tissue. It would be especially useful to have a biomarker that can identify those subjects whose prostate cancers are at an elevated risk for progression or transformation to life-threatening androgen-resistant or metastatic disease.

### SUMMARY OF THE INVENTION

The present invention relates generally to methods and uses of diagnosing, determining risk of developing, prognosing, monitoring treatment of, detecting, classifying and treating prostate cancer in a subject suspected of having or having prostate cancer by assessing the expression level of PCAT18. PCAT18 RNA is a long noncoding RNA identified herein as being differentially expressed in cancer calls, particularly in prostate cancer cells, as compared to normal prostate cells and as being specific for prostate cancer as compared to other neoplasms.

In an aspect, the present invention relates to method for diagnosing prostate cancer in a subject suspected of having prostate cancer comprising: (a) assessing the expression level of PCAT18 in a biological sample obtained from the subject; (b) comparing so the expression level of PCAT18 in the biological sample to a reference expression level; and (c) identifying the subject as having prostate cancer when the expression level of PCAT18 in the biological sample is greater than the reference expression level, or identifying the subject as not having prostate cancer when the expression level of PCAT18 in the biological sample is not greater than the reference expression level.

In another aspect, the present invention relates to a method for determining the risk of a subject for developing prostate cancer comprising: (a) assessing the expression level of PCAT18 in a biological sample obtained from the subject; (b) comparing the expression level of PCAT18 in the biological sample to a reference expression level; and (c) identifying the subject as having an increased risk of developing prostate cancer when the expression level of PCAT18 in the biological sample is greater than the reference expression level, or identifying the subject as not having an increased risk of developing prostate cancer when the expression level of PCAT18 in the biological sample is not greater than the reference expression level.

In another aspect, the present invention relates to a method for monitoring a treatment for prostate cancer in a subject diagnosed with prostate cancer comprising: (a) obtaining a baseline level by assessing the expression level of PCAT18 in a biological sample obtained from the subject prior to administration of the treatment; (b) administering the treatment to the subject for a treatment period; (c) after the treatment period, assessing the expression level of PCAT18 in a second biological sample obtained from the subject (d) comparing the expression level of PCAT18 in the second biological sample to the baseline level; and (e) identifying a poor response to the treatment when the expression level of PCAT18 in the second biological sample is greater than the baseline level, or identifying a good response to the treatment when the expression level of PCAT18 in the second biological sample is not greater than the baseline level.

The present invention further relates to a method for determining a prognosis of a subject diagnosed with having prostate cancer comprising: (a) assessing the expression level of PCAT18 in a biological sample obtained from the subject; (b) comparing the expression level of PCAT18 in the biological sample to a threshold expression level; and (c) determining a prognosis for the subject diagnosed with having prostate cancer based on the expression level of PCAT18 in the biological sample relative to the threshold expression level.

In yet another aspect, the present invention relates to a method for determining a risk of metastatic spread of prostate cancer in a subject diagnosed with prostate cancer comprising: (a) assessing the expression level of PCAT18 in a biological sample obtained from the subject (b) comparing the expression level of PCAT18 in the biological sample to a threshold expression level; and (c) identifying the subject as having an increased risk of metastatic spread when the expression level of PCAT18 in the biological sample is greater than the threshold expression level, or identifying the subject as not having an increased risk of metastatic spread when the expression level of PCAT18 in the biological sample is not greater than the threshold expression level.

In the methods described herein, the biological sample may be plasma, blood, serum, urine, saliva or tissue obtained from the subject. The tissue may comprise a cancerous prostate tissue sample, a benign prostatic hyperplasia tissue, or a normal prostate tissue.

Furthermore, the assessing of the expression level of PCAT18 in the biological samples obtained from subjects may be performed by evaluating the amount of PCAT18 RNA in the biological samples.

In another aspect the present invention relates to a method of treating a subject diagnosed with prostate cancer by administering a therapeutically effective amount of an inhibiting agent of PCAT18, wherein the inhibiting agent of PCAT18 is an antisense oligonucleotide, an siRNA, or a combination thereof.

The siRNA used in the method of treating described above may comprise an antisense nucleotide sequence corresponding to SEQ ID NO:22 or SEQ ID NO:23.

The antisense oligonucleotide used in the method of treating described above comprises a nucleotide sequence corresponding to SEQ ID NO:24 or SEQ ID NO:25.

In yet another aspect the present invention relates to a pharmaceutical composition comprising a therapeutic agent effective to reduce an amount of PCAT18 in cancerous prostate cells exposed to the therapeutic agent, and a pharmaceutically acceptable carrier, wherein the therapeutic agent is an antisense oligonucleotide, an siRNA, or a combination thereof.

The siRNA used in the pharmaceutical composition described above may comprise an antisense nucleotide sequence corresponding to SEQ ID NO:22 or SEQ ID NO:23.

The antisense oligonucleotide used in the pharmaceutical composition described above may comprise a nucleotide sequence corresponding to SEQ ID NO:24 or SEQ ID NO:25.

In another aspect, the present invention relates to a use of PCAT18 RNA for diagnosing prostate cancer in a subject suspected of having prostate cancer, wherein PCAT18 RNA comprises a nucleotide sequence corresponding to SEQ ID NO:1.

In another aspect, the present invention relates to a use of PCAT18 RNA for determining the risk of a subject in developing prostate cancer, wherein PCAT18 RNA comprises a nucleotide sequence corresponding to SEQ ID NO:1.

In yet another aspect the present invention relates to a use of PCAT18 RNA for monitoring a treatment for prostate cancer in a subject diagnosed with prostate cancer, wherein PCAT18 RNA comprises a nucleotide sequence corresponding to SEQ ID NO:1.

The present invention further relates to a use of an inhibiting agent of PCAT18 RNA for treating a subject diagnosed with prostate cancer, wherein PCAT18 RNA comprises a nucleotide sequence corresponding to SEQ ID NO: 1.

Accordingly, in broad terms, a novel use of a lncRNA (LOC728606), herein termed JUPITER or PCAT18, as a biomarker for diagnosis and prognosis of prostate cancer is provided.

In broad terms, a novel use of a lncRNA (LOC728606), herein termed JUPITER or PCAT18, as a target for development of therapies for treatment of prostate cancer (including but not limited to localized, invasive, androgen (castration) resistant and metastatic prostate cancer) is provided.

In an aspect of the present invention, a novel use of a lncRNA (LOC728606), herein termed JUPITER or PCAT18, as a biomarker for the early detection of prostate cancer provided.

In another aspect of the present invention, a novel use of a lncRNA (LOC728606), herein termed JUPITER or PCAT18, as a biomarker for the diagnosis of prostate cancer is provided.

In another embodiment of the present invention, a novel use of a lncRNA (LOC728606), herein termed JUPITER or PCAT18, as a biomarker for the prognosis of prostate cancer, whereby increased levels of JUPITER measured in samples obtained from a patient with prostate cancer is predictive of poorer disease outcome or increased risk of disease relapse is provided.

In an aspect of the present invention, a novel use of a lncRNA (LOC728606), herein termed JUPITER or PCAT18, is provided as a biomarker for assessment of the metastatic potential of a prostate tumour whereby measurement of increased levels (relative to sampling at earlier timepoints) of JUPITER in samples from a subject with prostate cancer is indicative of increased risk or potential for metastatic spread (metastasis).

In an aspect of the present invention, a novel use of a lncRNA (LOC728606), herein termed JUPITER or PCAT18, is provided as a biomarker for detection of prostate cancer at increased risk of progression to or that has already progressed to the stage of androgen (castration) resistant disease.

In another aspect of the present invention, a novel use of a lncRNA (LOC728606), herein termed JUPITER or PCAT18, is provided as a biomarker that may be used in combination with other prostate cancer biomarkers (including but not limited to PSA, PCGEM1, PCA3 etc.) in tests or methods for the detection, prognosis or treatment monitoring of prostate cancer.

In another embodiment of the present invention, a novel use of a lncRNA (LOC728606), herein termed JUPITER or PCAT18, as a biomarker useful in tests/assays for monitoring the outcome of patients with prostate cancer (treatment response) that are treated with curative intent is provided.

In another embodiment of the present invention, lncRNA (LOC728606), herein termed JUPITER or PCAT18, may be measured for use in the novel methods of the present invention, in patient samples including but not limited to prostate tumour tissue, benign prostatic hyperplasia tissue, normal prostate tissue, blood (including whole blood, serum or plasma), urine, saliva or other tissues.

In another aspect of the present invention, a novel use of a lncRNA as a prostate cancer biomarker (i.e. including but not limited to use of said biomarker for detection, diagnostic, prognostic or treatment-monitoring) or target for treatment of prostate cancer is provided, whereby the nucleotide sequence of the lncRNA is of about 90% or greater similarity to the sequence of JUPITER (LOC728606) (SEQ ID NO:1).

In another aspect of the present invention, a novel use of a lncRNA as a prostate cancer biomarker (i.e. Including but not limited to use of said biomarker for detection, diagnostic, prognostic or treatment-monitoring) or target for treatment of prostate cancer is provided, whereby the nucleotide sequence of the lncRNA is of about 95% or greater similarity to the sequence of JUPITER (LOC728606) (SEQ ID NO:1).

In another aspect of the present invention, a novel use of a lncRNA as a prostate cancer biomarker (i.e. including but not limited to use of said biomarker for detection, diagnostic, prognostic or treatment-monitoring) or target for treatment of prostate cancer is provided, whereby the nucleotide sequence of the lncRNA is of 99% or greater similarity to the sequence of JUPITER (LOC728606) (SEQ ID NO:1).

In yet another embodiment of the present invention, a novel use of any lncRNA as a prostate cancer biomarker (i.e. including but not limited to use of said biomarker for detection, diagnostic, prognostic or treatment-monitoring) or target for treatment of prostate cancer is provided, whereby the lncRNA comprises a contiguous nucleotide sequence of at least 200 base-pairs in length and whereby said lncRNA comprises a 200 base-pair (or longer) nucleotide sequence that is a fragment of the nucleotide sequence of JUPITER (LOC728606) (SEQ ID NO:1).

Further aspects of the invention will become apparent from consideration of the ensuing description of exemplary embodiments of the present invention. A person skilled in the art will realise that other embodiments of the invention are possible and that the details of the invention can be modified in a number of respects, all without departing from the inventive concept. Thus, the following drawings, descriptions and examples are to be regarded as illustrative in nature and not restrictive.

### BRIEF DESCRIPTION OF THE DRAWINGS

These and other features of the invention will become more apparent in the following detailed description in which reference is made to the appended drawings.

FIG. 1 (A) Hematoxylin-eosin staining of the prostate cancer xenograft (PCa Xenograft: left panels) and mouse lung tissue (Mouse Lung: right panels) of transplantable prostate cancer tumor lines LTL-313H and LTL-313B is shown. LTL-313H cells are more locally invasive to the adjacent kidney than LTL-313B cells, and show signs of distant metastatic spreading (never found in LTL-313B-engrafted mice). (B) Quantitative PCT (qPCR) confirmation of RNA sequencing data (columns represent average value, bars represent standard deviation, 2 replicate experiments). Values indicate relative expression level in LTL-313H vs. LTL-313B cells (i.e., the fold change of 313H/313B). The 4 most up-regulated transcripts were chosen (LOC728606, PCGEM1, H19, LINC461_1), along with 3 randomly selected transcripts (LOC285419, NCRNA116, LINC461_3). (C) Schematic representation of the PCAT18 locus (NLM “Gene” website). The gene is located in a region between 24,286 and 24,266 K (Chromosome 18 primary assembly). Lines represent introns, rectangles represent exons. Dotted lines represent a relative distance that is larger than the one shown in the schematic representation. Arrows represent transcription direction. The genes flanking the PCAT18 locus (AQP4, aquaporin-4; KCTD1, Potassium Channel Tetramerization Domain-Containing Protein 1) are shown. (D) ORF (open reading frame) finder output for PCAT18 sequence. Open Reading Frames are shown as shaded squares throughout the sequence. Each lane represents a possible reading frame. The software identified no ORF longer than 267 bp for a transcript longer than 2 Kb. Considering 6 possible reading frames, protein-coding regions could account for no more than 16% of the whole transcript.

FIG. 2 (A) PCAT18 expression in normal prostate (n=6) and prostate cancer PCa (n=7) samples (horizontal bar represents median value, vertical bars represent minimum and maximum value per group). Data are from the Cbio Cancer database. ***p<0.001 (2-tailed unpaired T test). Fold change: 2.3. (B) PCAT18 expression in normal prostate (n=29) and PCa (n=131) samples (horizontal bar represents median value, vertical bars represent minimum and maximum value per group). Data are from the Oncomine™ database (Compendia Bioscience, Ann Arbor, Mich.). ***p<0.001 (Oncomine Analysis). Fold change: 7.2. (C) Expression of PCAT18 in 12 benign tissues (GEO database, http://www.ncbi.nlm.nih.gov/geo/, study ID: HG-U95D, n=2 per tissue, horizontal bar represents mean value, vertical bars represent minimum and maximum value per group). *p<0.05 compared to prostate (ANOVA and Holm-Sidack's post-test). Fold Change: 2.78-8.75 (prostate cancer compared to other tissues). (D) Oncomine™ analyis of PCAT18 expression in 16 tumor tissues (median-centered values, bars represent minimum and maximum value per group). Data are centered to the median level of expression in the whole cohort. Sample size for each tumor type is in brackets: 1. Bladder Cancer (32); 2. Brain and CNS Cancer (4); 3. Breast Cancer (328): 4. Cervical Cancer (35), 5. Colorectal Cancer (330); 6. Esophageal Cancer (7); 7. Gastric Cancer (7); 8. Head and Neck Cancer (41); 9. Kidney Cancer (254); 10. Liver Cancer (11); 11. Lung Cancer (107); 12. Lymphoma (19); 13. Ovarian Cancer (166); 14. Pancreatic Cancer (19); 15. Prostate Cancer (59); 16. Sarcoma (49). ***p<0.001 (Oncomine™ Analysis).

FIG. 3 (A) PCAT18 expression (qPCR) in benign prostatic hyperplasia (BPH, n=5), low-Gleason PCa (n=5) and high-Gleason PCa (n=6) samples (Median-centered values, bars represent minimum and maximum value per group). ***p<0.001 (ANOVA and Tukey's post-test). (B) PCAT18 expression (qPCR) in plasma samples from normal individuals (normal) and from patients affected by localized PCa (Primary PCa) or metastatic castration-resistant PCa (MCRPC) (median-centered values, bars represent minimum and maximum value per group). **p<0.01 (ANOVA and Tukey's post-test). (C) PCAT18 expression in various prostate cancer cell lines (22RV1; LNCaP: human prostate cancer cell line, C4-2; PC3; and H660) relative to that in a benign prostatic hyperplasia cell line (BPH1). (D) siRNA-mediated PCAT18 silencing using two PCAT18-specific siRNAs (siRNA 1 and siRNA2) compared to a control. (E) Cell growth inhibition in the human prostate cancer cell line (C4-2) after specific silencing of PCAT18 expression using two PCAT18-specific siRNAs (siRNA 1 and siRNA2). Compared to a negative control (NC).

FIG. 4 (A) Transcripts positively associated with PCAT18 (Significance Analysis of Microarrays, SAM, analysis, Q<0.5%) were analyzed in Oncomine™ for “literature defined concepts” (threshold, p<1 E-4, odds ratio>2). Top 3 concepts associated with PCAT18-associated expression signature are shown (Down-Regulated genes in PCa after androgen ablation; Up-regulated genes in PCa in response to synthetic androgen R1881; and Up-regulated genes in prostate cancer cells in response to androgen). (B) Expression of 3 genes in xenografts from mice supplemented with Testosterone (Test) (2.5 mg/mouse, n=2) or after castration (1 week, 1 W.; 2 weeks, 2 W.; and 3 weeks 3 W. n=3). LOC728606 (PCAT18) down-regulation is comparable to PSA. Data are from LTL-331 xenografts human prostate cancer xenografts (www.livingtumorlab.com) and normalized to the average HPRT1 expression level in testosterone-supplemented animals. HPRT1 expression is stable pre- and post-castration (unpublished microarray data).

FIG. 5 shows the nucleotide sequence of the PCAT18 transcript (SEQ ID NO:1) (Entrez Gene ID: 728606; RefSeq ID: NR_024259.1).

FIG. 6 (A) PCAT18 expression levels in untreated LNCaP cells (Control) and cells supplemented with dihydrotestosterone (DHT, 10 nM, 6-24-48 h). LNCaP cells were grown in phenol red-free medium (RPMI-1640) supplemented with 10% charcoal-stripped FBS. Columns represent mean value (2 independent experiments performed in triplicate), bars standard deviation. (B), (C) The living tumor lab (www.livingtumorlab.com) comprises a collection of patient-derived PCa tumor tissue xenografts, originated with a method described in ref. An androgen-dependent PCa line (LTL313B) has been exposed to castrate-levels of testosterone for a prolonged time, in order to generate a castration-resistant subline. The figures show LTL313B tumor volume (B) and serum PSA levels (C) before and after castration. Neoplastic cells were implanted in male NOD/SCID intact mice, supplemented with testosterone until castration. Serum PSA was measured and mice were sacrificed for tumor volume measurement at indicated time points, as described before (Lin D, et al. High fidelity patient-derived xenografts for accelerating prostate cancer discovery and drug development. Cancer research. 2013). At 12-16 weeks post-castration, a castration-resistant, AR-positive cell line was generated (LTL-313BR). (D) PCAT18 expression was measured by qPCR in testosterone-supplemented. LTL313B, castrated xenografts (3 weeks) and in a CRPC sub-line (LTL313BR, no testosterone supplementation).

FIG. 7 (A) C4-2 invasion was quantified 24 h after the start of the invasion assay. Cells were transfected with 2 nM Negative Control (NC) or PCAT18-targeting siRNA1 and siRNA2. Columns represent mean value (4 experiments) bar SD. ***p<0.001 (ANOVA and Dunnet's post-test). (B) C4-2 cell migration was quantified at 6 h, 24 h or 48 h post-transfection. **P<0.01, ***P<0.001 (siRNA vs. NC), 2 way ANOVA and Tukey's post-test (C), (D), (E), MTT assay was performed on LNCaP (C) C4-2 (0) and BPH (E) cells treated with negative control (NC) or PCAT18-targeting siRNAs (both at 2 nM concentration) on days 1-3-5 post-transfection, as previously described (Watahiki A, et al. MicroRNAs associated with metastatic prostate cancer. PloS one. 2011; 6(9):e24950). Dots represent mean value, lines standard deviation (2 experiments performed in triplicate, data normalized to cell number in NC-day1) ***p<0.001 (2 way ANOVA and Tukey's post-test). (F) LNCaP cells were transfected with negative control (NC) or PCAT18-targeting siRNAs for 5 days. Bars represent mean values, lines standard deviations (2 independent experiments performed in triplicate). ***p<0.001 with respect to NC (ANOVA and Dunnet's post-test).

FIG. 8 (A) TaqMan qPCR confirmation of PCAT18 expression in PCa xenograft models. (B) TaqMan qPCR confirmation of PCAT18 expression in clinical samples. (C) Basal expression levels of PCAT18 in a panel of prostate cancer cell lines. Columns represent mean values, bars standard deviations (2 independent experiments). (D) Sub-cellular localization of PCAT18. GAPDH and MALAT1. Cellular (C) and Nuclear (N) RNA fractions where extracted and quantified by TaqMan assay, as described in methods section as of the Examples. Columns represent mean value, bars standard deviation (2 independent experiments). (E) TaqMan qPCR confirmation of siRNA-mediated PCAT18 silencing (C4-2 cells). Columns represent mean value, bars standard deviation (2 independent experiments).

FIG. 9 shows the nucleotide sequences of the antisense oligonucleotides. (A) shows the nucleotide sequence of antisense oligonucleotide (NC) with no known specific target in human or mouse genome (SEQ ID NO:26). (B) shows the nucleotide sequence of antisense oligonucleotide ASO2 (SEQ ID NO:24). (C) shows the nucleotide sequence of antisense oligonucleotide ASO7 (SEQ ID NO:25).

FIG. 10 shows the results of PCAT18 knockdown in C4-2 cells using antisense oligonucleotides ASO2 and ASO7 corresponding to SEQ ID NO:24 and SEQ ID NO:25, respectively, and using antisense oligonudeotide (NC) with no known specific target in human or mouse genome (corresponding to SEQ ID NO: 26). The columns and the bars represent mean value and standard deviation, respectively. ****p<0.0001 (ANOVA and Dunnetts's post-test).

### DETAILED DESCRIPTION OF THE INVENTION

Throughout the following description, specific details are set forth in order to provide a more thorough understanding of the present invention. However, the present invention may be practiced without these particulars. In other instances, well known elements have not been shown or described in detail to avoid unnecessarily obscuring the invention. Accordingly, the specification and drawings are to be regarded in an illustrative, rather than a restrictive sense.

The present invention relates to a long noncoding RNA (lncRNA) and methods and uses of the lncRNA for diagnosing, prognosing, monitoring and treating PCa.

lncRNAs may be transcribed from any genomic region, including, but not limited to, intergenic lncRNA or intervening non-coding RNA (lincRNA), which refers to lncRNA transcripts that are located between two protein-coding genes and transcribed from the + and/or −DNA strand(s); and intragenic lncRNA, which refers to lncRNA transcripts that are located within a protein-coding gene. Intragenic lncRNAs may be located within a coding region (i.e., an exon) of the gene and/or within a non-coding region (i.e., an intron) of the protein-coding gene, and transcribed from the + and/or −DNA strand(s).

Therefore, the present invention relates generally to identifying and characterizing long noncoding RNAs (“lncRNAs”) that are differentially expressed in cancer cells, particularly in prostate cancer cells, as compared to normal prostate cells. In particular, one such lncRNA, PCAT18 (Prostate Cancer-Associated Transcript-18; also referred to herein as JUPITER), located in the intergenic genomic region of chromosome 18q11.2, has been shown to be unregulated in cancerous cells found in the prostate.

As described herein, a multi-step profiling strategy was used to identify PCAT18 whose expression is: (1) significantly higher in PCa compared to 26 other benign and neoplastic tissues; (2) detectable in plasma samples; (3) able to discriminate between localized disease and mCRPC, as described further below.

### Definitions

Unless defined otherwise, all technical and scientific terms used herein have the same meaning as commonly understood by one of ordinary skill in the art to which this invention belongs.

As used herein, the term “about” refers to an approximately +/−10% variation from a given value. It is to be understood that such a variation is always included in any given value provided herein, whether or not it is specifically referred to.

The term “plurality” as used herein means more than one, for example, two or more, three or more, four or more, and the like.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements end/or steps, and in other embodiments consist of those elements and/or steps, whether or not these embodiments are specifically referred to.

As used herein, the term “localized prostate cancer” or “primary prostate cancer” refers to prostate cancer that is only in the prostate gland and has not metastasized or spread to another part of the body. An expression level of PCAT18 in a biological sample that is between about a 1.1 fold-change and about a 4 fold-change over the reference expression level, or any amount therebetween is indicative of primary prostate cancer.

The term “metastatic prostate cancer,” as used herein, refers to prostate cancer that has metastasized or spread outside the prostate gland to the lymph nodes, bones or other areas of the body. An expression level of PCAT18 in a biological sample that is greater than about a 4 fold-change over the reference expression level, for example greater than about 4 fold to about 1000 fold, or any amount therebetween is indicative of metastatic prostate cancer, or metastatic castration-resistant prostate cancer.

The term “metastatic castration-resistant prostate cancer” or “mCPRC,” as used herein, refers to prostate cancer that is resistant to medical (e.g., hormonal) or surgical treatments that lower testosterone, and has metastasized or spread to other parts of the body.

The progression of PCa may be classified using several methods including measuring PSA levels, Gleason Score, tumour stage typing, or a combination thereof (see for example, www.cancer.gov/cancertopics/treatment/prostate/understanding-prostate-cancer-treatment/page3). For example, low risk PCa may be defined as having a Gleason Score of 6 or lower (tumour stage T1 or T2a), a medium-risk PCa may be defined as having a Gleason Score of 7 (tumour stage T2b), and a high risk PCa may be defined as having a Gleason Score of 8 or higher (tumour stage T2c; Mazhar & Waxman. (2008) Nature Clinical Practice Urology 5: 486-493; D'Amico, et al. (1998) JAMA 280 (11):969-974). A low Gleason PCa, as used herein is characterized as having a Gleason Score of less than 6. A more aggressive PCa; as used herein is characterized as having a Gleason Score of 7 or greater than 7 (i.e. medium risk and high risk prostate cancer).

As used herein, an “expression level” of a transcript in a subject, for example, of the PCAT18 transcript, refers to an amount of transcript, such as PCAT18 RNA, in the subject's undiagnosed biological sample. The expression level may be compared to a reference expression level to determine a status of the sample. A subject's expression level can be either in absolute amount (e.g., number of copies/ml, nanogram/mil or microgram/ml) or a relative amount (e.g., relative intensity of signals; a percent or “fold” or “fold-change” increase).

A “reference level” or “reference expression level” (may also be considered a control), as used herein refers to an amount of the PCAT18 RNA or a range of amounts of the PCAT18 RNA measured in a normal individual or in a population of individuals without prostate cancer. For example, a reference expression level of the PCAT18 may be determined based on the expression level of PCAT18 in samples obtained from normal individuals. A reference expression level can be either in absolute amount (e.g., number of copies/ml, nanogram/ml or microgram/ml) or a relative amount (e.g., relative intensity of signals: a percent or “fold” or “fold-change” increase).

As used herein, a “threshold level” or “threshold expression level” refers to an expression level of PCAT18 in a biological sample that is between about a 1.1 fold-change and about a 4 fold-change over the reference expression level, or any amount therebetween. A threshold expression level is indicative of localized prostate cancer or primary prostate cancer.

As used herein, a “baseline level” refers to an expression level of PCAT18 in a first biological sample obtained from a subject that is determined prior to any treatment or during any treatment, and is used as comparison to a second expression level of PCAT18 that is assessed from a second biological sample that is obtained from the subject at a time after the first biological sample is obtained. This baseline level may be used, for example, without limitation, in monitoring the progression of PCa in a subject, monitoring a treatment regimen or treatment modality in a subject having PCa, determining whether a treatment regimen or treatment modality should be considered in a subject, determining whether a treatment regimen or treatment modality should be discontinued in a subject, or determining whether a treatment regimen or treatment modality should be modified in a subject.

As used herein, “normal individual” refers to an individual that has been tested for prostate cancer using a combination of diagnostic methods, including T stage, Gleason grade, plasma PSA levels and PCAT18 expression levels and determined to not have prostate cancer by a physician.

The term “gene,” as used herein, refers to a segment of nucleic acid that encodes an RNA, which RNA can be a coding or noncoding RNA.

The term “selectively hybridize.” as used herein, refers to the ability of a particular nucleic acid sequence to bind detectably and specifically to a second nucleic acid sequence. Selective hybridization generally takes place under hybridization and wash conditions that minimize appreciable amounts of detectable binding to non-specific nucleic acids. High stringency conditions can be used to achieve selective hybridization conditions as known in the art and discussed herein. Typically, hybridization and washing conditions are performed at high stringency according to conventional hybridization procedures with washing conditions utilising a solution comprising 1-3×SSC, 0.1-1% SOS at 50-70° C., with a change s of wash solution after about 5-30 minutes.

The term “identity” or “% identical” as used herein refers to the measure of the identity of sequence between two nucleic acids molecules. Identity can be determined by comparing a position in each sequence which may be aligned for purposes of comparison. Two nucleic acid sequences are considered substantially identical if they share at least about 80% sequence identity or at least about 81% sequence identity, or at least about 82% sequence identity, or at least about 83% sequence identity, or at least about 84% sequence identity, or at least about 85% sequence identity, or at least about 86% sequence identity, or at least about 87% sequence identity, or at least about 88% sequence identity, or at least about 89% sequence identity, or at least about 90% sequence identity. Alternatively, two nucleic acid sequences are considered substantially identical if they share at least about 91% sequence identity, or at least about 92% sequence identity, or at least about 93% sequence identity, or at least about 94% sequence identity, or at least about 95% sequence identity, or at least about 96% sequence identity, or at least about 97% sequence identity, or at least about 98% sequence identity, or at least about 99% sequence identity.

Sequence identity may be determined by the BLAST algorithm which was originally described in Altschul et al. (1990) J. Mol. Biol. 215:403-410. The BLAST algorithm may be used with the published default settings. When a position in the compared sequence is occupied by the same base, the molecules are considered to have shared identity at that position. The degree of identity between sequences is a function of the number of matching positions shared by the sequences and the degree of overlap between the sequences. Furthermore, when considering the degree of identity with SEQ ID NO:1 or a contiguous portion of SEQ ID NO:1, it is intended that the equivalent number of nucleotides be compared to SEQ ID NO:1 or the contiguous portion of SEQ ID NO:1, respectively Additional sequences outside of those being compared are not intended to be considered when determining the degree of identity with. The sequence identity of a given sequence may be calculated over the length of the reference sequence (i.e., SEQ ID NO:1 or the contiguous portion of SEQ ID NO:1).

The terms “corresponding to” or “corresponds to” indicate that a polynucleotide sequence is identical to all or a portion of a reference polynucleotide sequence. In contradistinction, the term “complementary to” is used herein to indicate that the polynucleotide sequence is identical to all or a portion of the complementary strand of a reference polynucleotide sequence. For illustration, the nucleotide sequence “TATAC” corresponds to a reference sequence “TATAC” and is complementary to a reference sequence “GTATA.”

The term “target gene,” as used herein, refers to the gene the expression of which is to be modulated with a siRNA molecule or ASO molecule or other inhibiting agent of the present invention. In the context of the present invention, the target gene is the lncRNA locus LOC728606 or the PCAT8 gene.

The term “target RNA.” as used herein refers to the RNA transcribed from a target gene.

The term “antisense strand” refers to a nucleotide sequence that is complementary to the nucleotide sequence corresponding to SEQ ID NO:1 or that is complementary to a contiguous nucleotide sequence of a portion of the nucleotide sequence corresponding to SEQ ID NO:1. The term “sense strand” refers to a nucleotide sequence that corresponds to SEQ ID NO:1 (or a contiguous nucleotide sequence of a portion of the nucleotide sequence corresponding to SEQ ID NO:1) and thus is complementary to the antisense strand.

The terms “therapy,” and “treatment.” as used interchangeably herein, refer to an intervention performed with the intention of improving a recipients status. The improvement can be subjective or objective and is related to the amelioration of the symptoms associated with, preventing the development of, or altering the pathology of a disease, disorder or condition being treated. Thus, the terms therapy and treatment are used in the broadest sense, and include the prevention (prophylaxis), moderation, reduction, and curing of a disease, disorder or condition at various stages. Prevention of deterioration of a recipient's status is also encompassed by the term. Those in need of therapy/treatment include those already having the disease, disorder or condition as well as those prone to, or at risk of developing, the disease, disorder or condition and those in whom the disease, disorder or condition is to be prevented. In the context of the present invention, the disease, disorder or condition is prostate cancer, including benign prostate cancer, localized prostate cancer, indolent prostate cancer, mCRPC and other stage of prostate cancer.

The term “subject” or “patient,” as used herein, refers to a mammal in need of treatment.

The term “effective amount” as used herein refers to an amount of a compound that produces a desired effect. For example, a population of cells may be contacted with an effective amount of a compound to study its effect in vitro (e.g., cell culture) or to produce a desired therapeutic effect ex vivo or in vitro. An effective amount of a compound may be used to produce a therapeutic effect in a subject, such as preventing or treating a target condition, alleviating symptoms associated with the condition, or producing a desired physiological effect. In such a case, the effective amount of a compound is a “therapeutically effective amount.” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the compound (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the compound is administered alone or in combination with another compound, drug, therapy or other therapeutic method or modality. One skilled in the clinical and s pharmacological arts will be able to determine an effective amount or therapeutically effective amount through routine experimentation, namely by monitoring a cell's or subject's response to administration of a compound and adjusting the dosage accordingly. For additional guidance, see Remington: The Science and Practice of Pharmacy, 21st Edition, Univ. of Sciences in Philadelphia (USIP), Lippincott Williams & Wilkins, Philadelphia, Pa., 2005, which is hereby incorporated by reference as if fully set forth herein.

The term “in combination” or “in combination with.” as used herein, means in the course of treating the same disease in the same patient using two or more agents (including other siRNA or other ASO), drugs, treatment regimens, treatment modalities or a combination thereof, in any order. Administration of a PCAT18 siRNA or an ASO “in combination with” one or more other anti-cancer therapeutics or chemotherapeutics is intended to include simultaneous (concurrent) administration and consecutive administration, as well as administration in a temporally spaced order of up to several days apart. Consecutive administration is intended to encompass administration of the other therapeutic(s) and the siRNA molecule(s) and/or the ASO molecule(s) to the subject in various orders. Such combination treatment may also include more than a single administration of any one or more of the agents, drugs, treatment regimens or treatment modalities. Further, the administration of the two or more agents, drugs, treatment regimens, treatment modalities or a combination thereof may be by the same or different routes of administration.

A “biological sample” refers to any material, biological fluid, tissue, or cell obtained or otherwise derived from a subject including, but not limited to, blood (including whole blood, leukocytes, peripheral blood mononuclear cells, plasma, and serum), sputum, mucus, nasal aspirate, urine, semen, saliva, meningeal fluid, lymph fluid, milk, bronchial aspirate, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample may be a combination of samples from an individual, such as a combination of a tissue and fluid sample. A biological sample may also include materials containing homogenized solid material, such as from a stool sample, a tissue sample, or a tissue biopsy; or materials derived from a tissue culture or a cell culture. Tissue may be normal tissue or cancerous tissue, such as a cancerous prostate tissue, a benign prostatic hyperplasia tissue, or normal prostate tissue.

### Identification and Expression Analysis of PCAT18

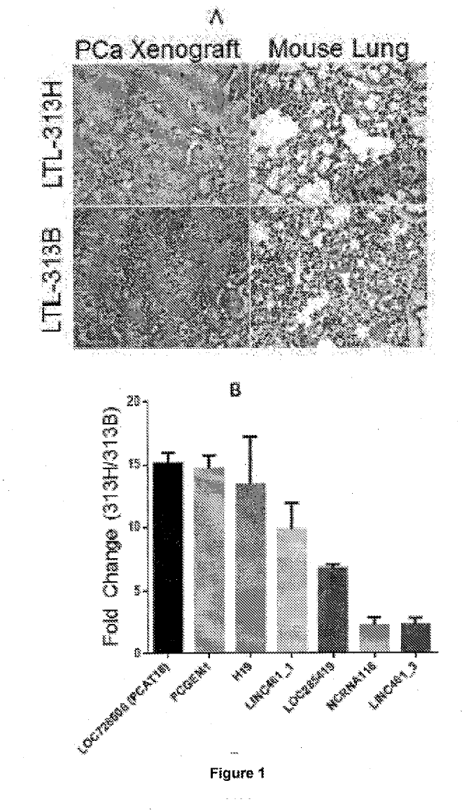

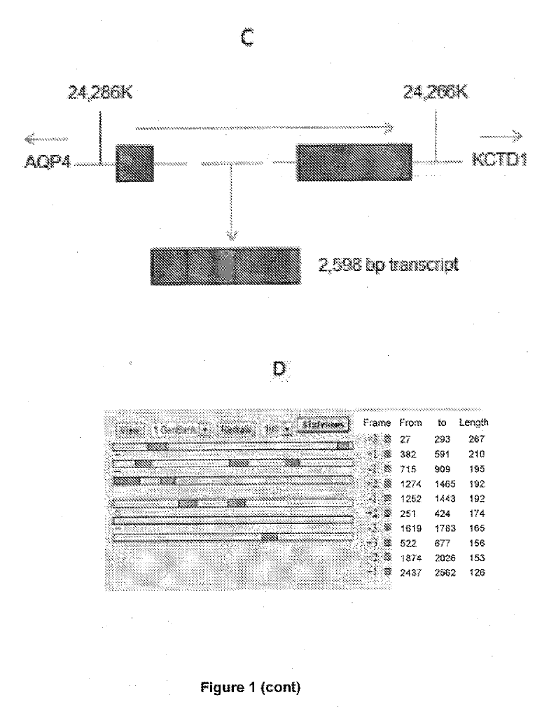

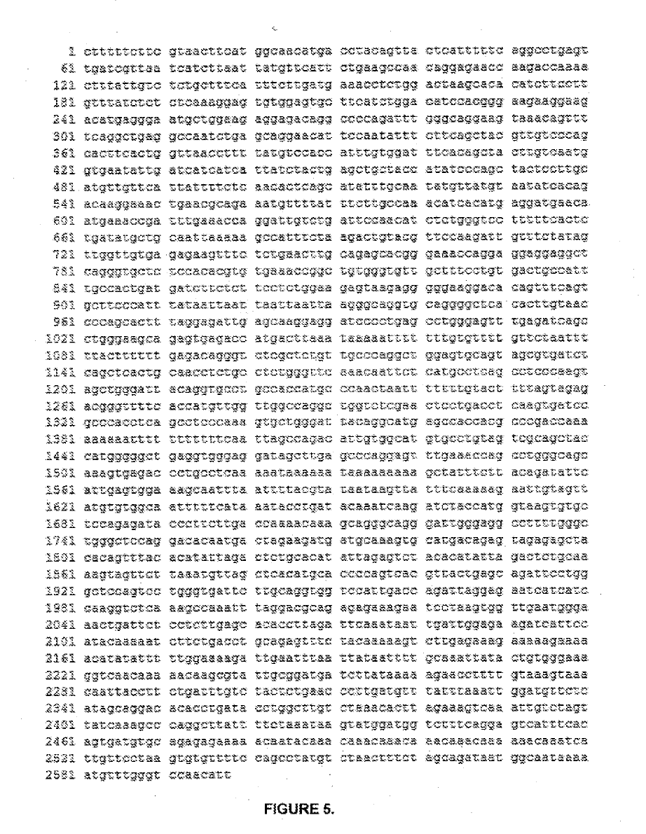

PCAT18 (SEQ ID NO:1; see FIG. 5) is a long intergenic noncoding RNA at locus LOC728606, exhibiting high expression in a metastatic xenograft model (see Example 1). PCAT18 showed a similar magnitude of fold-change as the oncogenic lncRNAs H19 and PCGEM1 (see FIG. 1(B)).

Locus LOC728606, which encodes the intergenic lncRNA PCAT18, is flanked by AQP4 (Aquaporin-4) and KCTD1 (Potassium channel tetramerisation domain containing-1) loci and is part of the 18q11.2 genomic locus. PCAT18 is a 2598 bp RNA containing 2 exons (FIG. 1(C)) and consists of the nucleotide sequence referenced as SEQ ID NO:1 (FIG. 5).

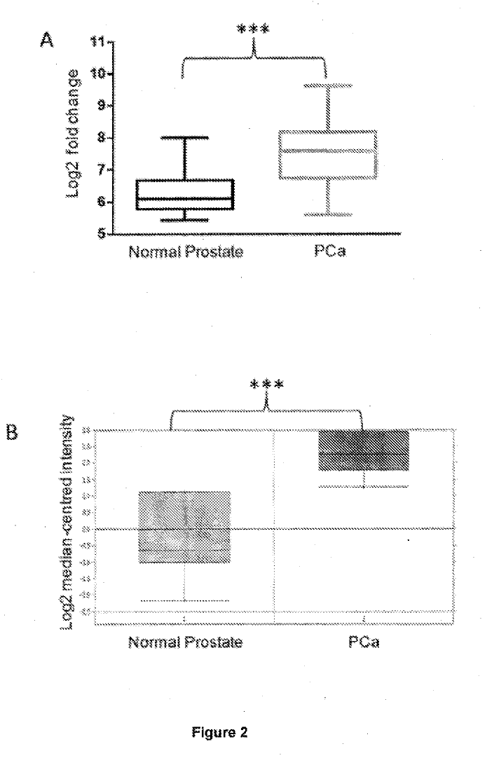

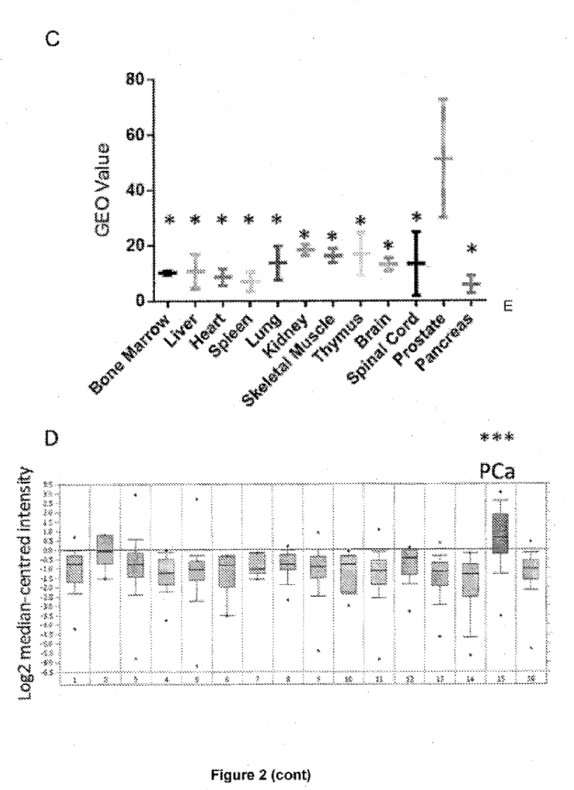

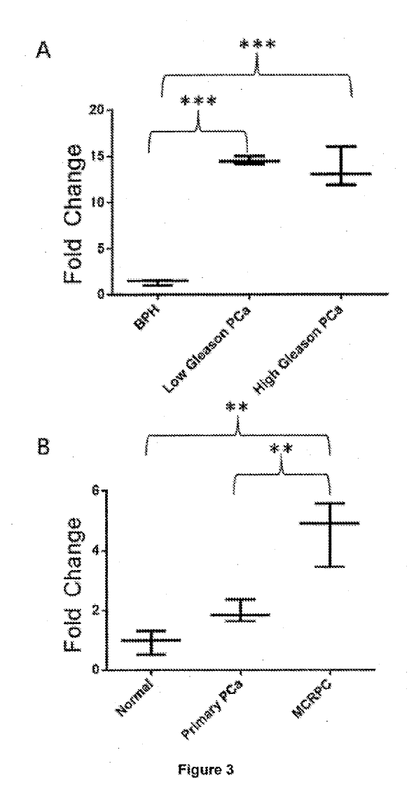

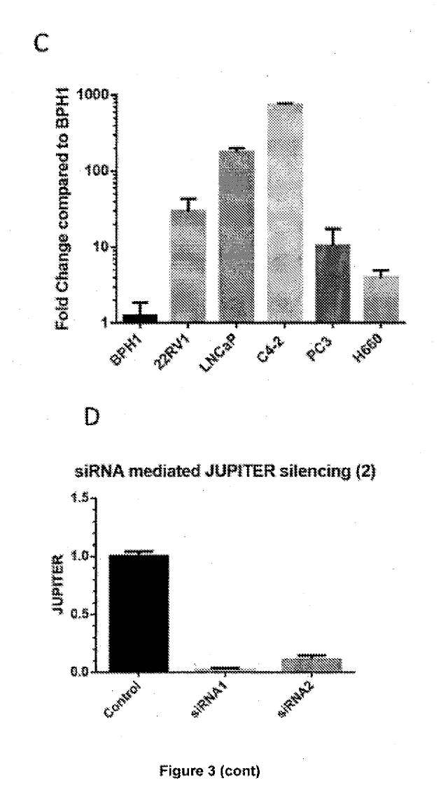

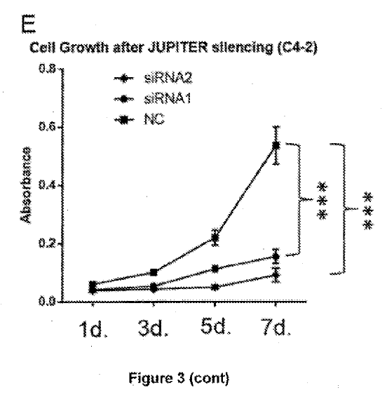

Expression analysis of PCAT18 using publically available databases (i.e., Oncomine™, Geo and cBio database) showed up-regulation of PCAT18 in PCa as compared to normal tissues (see FIGS. 2(A) and (B). Using quantitative PCR (QPCR), it was further determined that PCAT18 is highly over-expressed (8.8-11.1 fold, p<0.001) in both low-Gleason (i.e., a lower grade PCa; Gleason Score of 6 or lower) and high-Gleason PCa (i.e., more aggressive PCa; Gleason Score of 7, or higher than 7) samples as compared to benign prostatic hyperplasia (see FIGS. 3(A) and (B)). A person skilled in the art would recognize that this up-regulation is, thus, not merely the function of prostate cell hyperproliferation.

Expression of PCAT18 is significantly higher in normal prostate tissue than in normal tissues (see FIG. 2(D)) and that it is over-expressed specifically in PCa as compared to 15 other neoplastic tissues (FIG. 2(C)), including, bladder cancer, brain and central nervous system cancer, breast cancer, cervical cancer, colorectal cancer, esophageal cancer, gastric cancer, head and neck cancer, kidney cancer, liver cancer, lung cancer, lymphoma, ovarian cancer, pancreatic cancer, and sarcoma. PCAT18 is, therefore, prostate cancer-specific and prostate tissue-specific, which suggests its usefulness as a biomarker for disease detection, diagnosis and monitoring of PCa and for treatment of PCa, as indicated for other noncoding RNAs.

Patients affected by mCRPC had significantly higher levels of PCAT18 in their plasma samples as compared to patients affected by localized PCa (see FIG. 3(B)). In addition, those patients affected by localized PCa had significantly higher levels of PCAT18 in their plasma samples when compared to plasma samples from normal individuals. Accordingly, those plasma samples derived from patients having a poor prognosis showed a tendency toward having higher levels of PCAT18 as compared to plasma samples derived from patients having a good or better prognosis than the patients having a poor prognosis. This finding also indicates that PCAT18 is detectable in plasma samples and could provide a non-invasive method for diagnosis, prognosis and monitoring of PCa and treatment of PCa.

In addition to the PCAT18B transcript described above (i.e., FIG. 5, SEQ ID so NO:1), one skilled in the art would understand that many additional transcript variants of PCAT18 may also exist and could be applicable for the uses and methods described herein. For example, one or more transcript variants may include, without limitation, a variant that is at least about 90% identical, at least about 91% identical, at least about 92% identical, at least about 93% identical, at least about 94% identical, at least about 95% identical, at least about 96% identical, at least about 97% identical, at least about 98% identical, at least about 99% identical to SEQ ID NO:1. Methods of determining sequence identity are known in the art and can be determined, for example, by using the BLASTN program of the University of Wisconsin Computer Group (GCG) software or provided on the NCBI website. The PCAT18 transcript variants that may be used in accordance with the methods described herein are not limited to those described above. The transcript variants may include any addibonal variants of PCAT18 described herein and other lncRNAs that are transcribed from genomic locus LOC728606, as one skilled in the art would understand that many additional transcript variants related to PCAT18 may exist that have differential expression found in prostate cancer cells as compared to normal cells.

In certain embodiments, the transcript variant is capable of selectively hybridizing under stringent conditions to a portion of the genomic region at locus LOC728606. Suitable stringent conditions include, for example, hybridization according to conventional hybridization procedures and washing conditions of 1-3×SSC, 0.1-1% SDS, 50-700 C with a change of wash solution after about 5-30 minutes. As known to those of ordinary skill in the art, variations in stringency of hybridization conditions may be achieved by altering the time, temperature, and/or concentration of the solutions used for the hybridization and wash steps. Suitable conditions can also depend in part on the particular nucleotide sequences used.

It is understood that modifications of the PCAT18 lncRNA may also be used as a biomarker for detecting, prognosing and monitoring cancer according to the methods and uses described herein. Modifications of PCAT18 transcripts that may be detected and that may be indicative of PCa when used according to the methods and uses described herein may include, but are not limited to, single nucleotide polymorphisms (SNPs), DNA methylation or unmethylation. RNA methylation or unmethylation, and gene mutations or deletions. Such modifications may result in an alteration in the expression, formation, or conformation of the PCAT18 transcript in a cancerous or biological sample, as compared to a control, and may result in inhibition or impairment of a therapeutic agent targeting such PCAT18 transcript. Alternatively, downstream targets of the PCAT18 transcript may be used as biomarkers for detecting, prognosing and monitoring cancer according to the methods described herein.

As used herein “PCAT18 transcript” or “PCAT18” or “JUPITER” may be PCAT18 RNA comprising the nucleotide sequence referenced as SEQ ID NO:1, a variant transcript of PCAT18, as described above, comprising from about 90% to about 100%, or any amount therebetween, identity or sequence similarity with SEQ ID NO:1, or a modification of either PCAT18 or a related transcript, or may be any other lncRNA that is transcribed from genomic locus LOC728606, which has increased expression in prostate cancer cells as compared to normal cells.

### Methods of Diagnosis or Prognosis Using PCAT18

PCAT18, and/or one or more of the individual PCAT transcript variants, may be isolated from a biological sample (e.g., blood, serum, plasma, urine, saliva or prostate tissue) and the expression level of PCAT18 assessed in the biological sample to determine a diagnosis or prognosis of PCa and any stage of PCa. As described above, the expression of PCAT18 is tissue-specific (i.e., prostate tissue) and cancer-specific (PCa), with overexpression of PCAT18 in biological samples obtained from patients having PCa.

As described herein, the expression levels of PCAT18 in a biological sample (such as plasma, urine, tissue, saliva) show a progressive increase from non-cancerous (i.e., a sample from a normal individual) to primary PCa to metastatic PCa. The more aggressive form of prostate cancer (mCRPC) exhibit the highest levels of PCAT18, as compared to primary, localized PCa and normal individuals. Accordingly, the expression level of PCAT18 in a biological sample is associated with clinical aggression of PCa and, consequently, patient survival from PCa. Therefore, according to certain embodiments, the PCAT18B transcript is associated with the presence or absence of primary PCa, metastatic PCa, including mCRPC, local or distant metastases, and the progression or aggressiveness of the PCa. As the expression levels of PCAT18 is also associated with patient survival, PCAT18 and other variants of PCAT18 and transcripts of the LOC728606 genomic locus may be used as biomarkers for diagnosing, prognosing, assessing risk and monitoring PCa. Further, such diagnoses, prognoses and assessments of risk of PCa based on expression levels of PCAT18 transcripts and related variants may be used to monitor a PCa patient's treatment and/or make clinical decisions regarding optimization of a PCa patient's treatment regimen.

Therefore, the present invention relates to methods for diagnosis of a subject suspected of having prostate cancer, which involves assessing or determining PCAT18 expression levels in a biological sample obtained from the subject and comparing the expression level to a reference expression level. The reference expression level may be obtained from the expression level of PCAT18 in samples obtained from normal individuals determined as not having PCa. Such methods further include a step of diagnosing a subject as having PCa or identifying the subject as having PCa when the expression level of PCAT18 in the biological sample of the subject is greater than the reference expression level. The subject may also be diagnosed as not having PCa or identified as not having PCa when the expression level of PCAT18 in the biological sample of the subject is not greater than a reference expression level. Depending on the level of expression of PCAT18 in the biological sample as compared to the reference expression level, the subject may be diagnosed with localized prostate cancer or a metastatic prostate cancer, including, without limitation, metastatic castration-resistant prostate cancer (mCRPC). For example, if the expression level of PCAT18 in a subject's sample is between about a 1.1 fold-change and about a 4 fold-change, or any amount therebetween, over the reference expression level, then the subject may be diagnosed with a localized prostate cancer, and if the expression level of PCAT18 in a subject's sample is greater than about a 4 fold-change, for example greater than 4 to about 1000 fold or any amount therebetween, over the reference expression level, then the subject may be diagnosed with a clinically more aggressive prostate cancer, for example, without limitation, mCRPC. In certain embodiments, the diagnostic methods described herein may detect, determine, or recognize the presence or absence of PCa; prediction or diagnosis of metastasis or lack of metastasis, type or sub-type, or other classification or characteristic of PCa; whether a specimen is a benign lesion, such as benign prostatic hyperplasia (BPH), or a malignant tumor, or a combination thereof.

By “greater than the reference expression level,” it is meant that the expression level of PCAT18 in the subject's sample is at least about a 1.5 increase or fold-change over the reference expression level, for example, the expression level of PCAT18 may be at least about a 2 fold-change (as shown, for example, in FIG. 3(B)), at least about a 2.3 fold-change (as shown, for example, in FIG. 2(A)), at least about a 2.78 fold-change (as shown, for example, in FIG. 2(C)), at least about a 3 fold-change, at least about a 3.5 fold-change, at least about a 4 fold-change, at least about a 4.5 fold-change, at least about a 5 fold-change, at least about a 5.5 fold-change (as shown in, for example, FIG. 3(B)), at least about a 6 fold-change, at least about a 6.5 fold-change, at least about a 7 fold-change, at least about a 7.2 fold-change (as shown, for example, in FIG. 2(B)), at least about a 7.5 fold-change, at least about an 8 fold-change, at least about an 8.5 fold-change, at least about an 8.75 fold-change (as shown, for example, in FIG. 2(C)), at least about a 9 fold-change, at least about a 9.5 fold-change, at least about a 10 fold-change, at least about a 11 fold-change, at least about a 12 fold-change, at least about a 13 fold-change, at least about a 14 fold-change (as shown, for example, in FIG. 3(A)), at least about a 15 fold-change (as shown, for example, in FIG. 3(A)), at least about a 16 fold-change, at least about a 17 fold-change, at least about an 18 fold-change, at least about a 19 fold-change, at least about a 20 fold-change, at least about a 30 fold-change, at least about a 40 fold-change, at least about a 50 fold-change, at least about a 60 fold-change, at least about a 70 fold-change, at least about an 80 fold-change, at least about a 90 fold-change, at least about a 100 fold-change, at least about a 200 fold-change, at least about a 300 fold-change, at least about a 400 fold-change, at least about a 500 fold-change, at least about a 600 fold-change, at least about a 700 fold-change, at least about an 800 fold-change, at least about a 900 fold-change, at least about a 1000 fold-change, or any other fold-change therebetween, over the reference expression level. Furthermore, the change in expression may be from about 1.5 to about 150, fold increase or fold-change, or any amount therebetween, over the reference expression level (as shown in FIG. 3C: LNCap), or from about 1.5 to about 1000 fold increase or fold-change, or any amount therebetween, over the reference expression level (as shown in FIG. 3C: C4-2). For example, the increase in expression of PCAT18 over the reference expression level may be about 1.5, 2, 4, 6, 8, 10, 20, 25, 50, 75, 100, 150, 200, 250, 300, 350, 400, 450, 500, 550, 600, 650, 700, 750, 800, 850, 900, 950, 1000 fold increase or fold-change, or any amount therebetween, over the reference expression level.

By “not greater than the reference expression level,” it is meant that the expression level of PCAT18 in the subject's sample is less than about a 1.4 increase or fold-change over the reference expression level, for example, the expression level of PCAT18 is less than about a 1.3 fold-change, less than about a 1.2 fold-change, less than about a 1.1 fold-change, less than about a 1 fold-change, less than about a 0.8 fold-change, less than about a 0.5 fold-change, less than about a 0.2 fold-change, less than about a 0.1 fold-change over the reference expression level. The phrase “not greater than the reference expression level” may also include situations in which the expression level of PCAT18 in the subject's sample is the same as or less than the reference expression level.

The present invention further relates to methods for determining the risk of a subject for developing prostate cancer. Such methods comprise a step of assessing or determining the expression level of PCAT18 in a biological sample obtained from the subject and comparing the expression level to a reference expression level. The reference expression level may be obtained from the expression level of PCAT18 in samples obtained from normal individuals determined as not having PCa. Such methods further include a step of identifying the subject as having an increased risk of developing PCa when the expression level of PCAT18 in the biological sample of the subject is greater than the reference expression level. The subject may also be identified as not having an increased risk of developing PCa when the expression level of PCAT18 in the biological sample of the subject is not greater than a reference expression level. By “increased risk of developing PCa,” it is meant a greater than about a 10% chance of developing PCa as compared to a normal individual, for example, a greater than about a 15%, about a 20%, about a 25%, about a 30%, about a 35%, about a 40%, about a 45% or about a 50% chance of developing PCa as compared to a normal individual.

PCAT18 expression levels in a biological sample from a subject may also be used in the prognosis of a PCa patient (i.e., a subject having PCa), which involves assessing or determining PCAT18 expression levels in a biological sample obtained from the subject and comparing the expression level to a threshold level. Such methods described herein may, therefore, include a step of determining a prognosis for a subject having PCa when an expression level of PCAT18 is greater than, less than or within the threshold level. The prognosis may refer to a prediction of a future course of PCa in a subject who has the disease or condition (e.g., predicting disease outcome, such as, but not limited to, predicting patient survival), and may also encompass the evaluation of the response or outcome of the disease in the individual after administering a treatment or therapy to the individual, and may refer to a prediction of an increased or reduced risk of PCa relapse. The prognosis may be a poor prognosis or a good prognosis, as measured by a decreased length of survival or a prolonged (or increased) length of survival, respectively. The prognosis may be a poor prognosis if the expression level of PCAT18 in the subject's biological sample is greater than the threshold level; that is, if the expression level of PCAT18 in the subject's biological sample is greater than about a 4 fold-change over the reference expression level. The prognosis may be good if the expression level of PCAT18 in the subject's biological sample is within the threshold level; that is, if the expression level of PCAT18 is between about a 1.1 fold-change and about a 4 fold-change over the reference expression level. The prognosis may be even better if the expression level of PCAT18 in the subject's biological sample is less than the threshold level.

In other embodiments, the methods described herein may also be used to differentiate between an early stage cancer (i.e., primary tumor); or a metastasized PCa when the expression level is significantly different than threshold level. Accordingly, a method for determining a risk of metastatic spread of (i.e. risk of metatsis in other organs or parts of the body that can be determined using standard tests) PCa in a subject diagnosed with PCa is provided herein. Such a method involves assessing or determining the expression level of PCAT18 in a biological sample obtained from the subject diagnosed with PCa and comparing the expression level to a threshold level. The subject is identified as having an increased risk of metastatic spread when the expression level of PCAT18 in the subject's biological sample is significantly greater than the threshold level. By “significantly greater than the threshold level,” it is meant that the expression level of the PCAT18 in the subject's biological level is at least about a 6 fold-change over a reference expression level, and may be about a 7 fold-change, about an 8-fold-change, about a 9 fold-change, about a 10 fold-change, about an 11 fold-change, about a 12 fold-change, about a 13 fold-change, about a 14 fold-change, about a 15 fold-change, about a 16 fold-change, about a 17 fold-change, about an 18 fold-change, about a 19 fold-change, about a 20 fold-change, about a 30 fold-change, about a 40 fold-change, about a 50 fold-change, about a 60 fold-change, about a 70 fold-change, about an 80 fold-change, about a 90 fold-change, about a 100 fold-change, about a 200 fold-change, about a 300 fold-change, about a 400 fold-change, about a 500 fold-change, about a 600 fold-change, about a 700 fold-change, about an 800 fold-change, about a 900 fold-change, about a 1000 fold-change over a reference expression level, or any amount therebetween of the expression level of PCAT18 to a reference level. By “increased risk of metastatic spread,” it is m(eant a greater than about a 10% chance of metastatic spread as compared to a normal individual, for example, a greater than about a 15%, about a 20%, about a 25%, about a 30%, about a 35%, about a 40%, about a 45% or about a 50% chance of metastatic spread as compared to a normal individual. The increased risk of metastatic spread includes, for example, without limitation, an increased risk of a locoregional metastasis, a distant metastasis or an increased risk of progression to a more clinically aggressive PCa, including, mCRPC.

The present invention further relates to methods for monitoring a treatment administered to a patient diagnosed with PCa and involves analyzing the expression level of PCAT18 at two different timepoints, such as prior to administration of treatment and after administration of treatment. Accordingly, the method comprises obtaining a baseline level of expression of PCAT18 in a biological sample obtained from the subject. This baseline level is obtained prior to administration of a treatment, or prior to a second timepoint at which an expression level of PCAT18 will be determined. The method then comprises the step of administering the treatment for a treatment period and then determining or assessing the expression level at a second timepoint from a second biological sample obtained from the subject. A comparison of the expression level at the second timepoint to the baseline level will identify whether the patient has responded poorly to the treatment or whether the patient has had a good response to the treatment. The second timepoint may also be after a certain period of time has elapsed from obtaining the first biological sample from the subject, without a treatment step in between. This may be the case if the method comprises a step of determining whether a treatment course, treatment regimen or treatment modality should be started, for example, if the patient's PCa was in remission and determining whether there has been a relapse in the patient, or if the patient's disease has progressed to mCRPC and determining whether surgery or hormonal therapy should be administered.

Accordingly, the methods described herein may also include monitoring or assessing the progression of PCa in a subject monitoring or assessing a response to treatment in a subject having PCa; monitoring or assessing a metastatic spread of PCa in a subject monitoring or assessing a remission state or a recurrence of PCa in a subject or a combination thereof. Such monitoring or assessing may include an individuals response to a therapy, such as, for example, predicting whether an individual is likely to respond favorably to a therapeutic agent, is unlikely to respond to a therapeutic agent, or will likely experience toxic or other undesirable side effects as a result of being administered a therapeutic agent; selecting a therapeutic agent for administration to an individual, or monitoring or determining an individual's response to a therapy that has been administered to the individual.

An expression level of PCAT18 in a subject or a reference expression level used in the methods for diagnosis, prognosis, monitoring, treating, or assessing risk of developing PCa or progression to metastatic risk, as described herein, may be measured, quantified and/or detected by any suitable RNA detection, quantification or sequencing methods known in the art, including, but not limited to, quantitative PCR (QPCR) or quantitative/gel-based electrophoresis PCR reverse transcriptase-polymerase chain reaction (RT-PCR) methods, microarray, serial analysis of gene expression (SAGE), next-generation RNA sequencing (e.g., deep sequencing, whole transcriptome sequencing, exome sequencing), gene expression analysis by massively parallel signature sequencing (MPSS), immune-derived colorimetnc assays, in situ hybridization (ISH) formulations (colorimetric/radiometric) that allow histopathology analysis, mass spectrometry (MS) methods, RNA pull-down and chromatin isolation by RNA purification (ChiRP), and proteomics-based identification (e.g., protein array, immunoprecipitation) of lncRNA. In an embodiment, the method of measuring the expression level of PCAT18 may also include non-PCR-based molecular amplification methods for detection. A combination of the above methods for assessing the expression level or reference expression level is also contemplated.

A diagnosis or prognosis of PCa based on the methods described herein may be used to optimize or select a treatment regimen for a subject diagnosed with PCa. For example, a method for diagnosing or prognosing PCa may be performed as described above. In an embodiment, a subject that is diagnosed with primary PCa based on an expression level of PCAT18 or a related variant may be treated according to FDA approved protocols and standards known in the art for a particular therapeutic agent for primary PCa. Alternatively, a diagnosis of primary PCa may be treated using surgery, such as, but not limited to, radical prostatectomy, and/or primary PCa may be treated using a “wait and see approach” before or after surgery, since such a diagnosis indicates that metastasis of the primary PCa has not occurred. In other embodiments, primary PCa may be treated using a therapeutic or pharmaceutical agent that targets PCAT18 and/or one or more related variants, and inhibits or silences the expression of PCAT18, as described below. After any of the above treatments, monitoring of the PCa and progression of the PCa to a more clinically aggressive PCa, such as mCRPC, is performed by periodically assessing the expression levels of PCAT18 and/or any related variants in a patient's biological sample, such as blood, plasma, urine or prostate tissue.

Similarly, a diagnosis of a subject that is diagnosed with metastatic PCa based on an expression level of PCAT18 or a related variant may be treated more aggressively according to FDA approved protocols and standards known in the art for metastatic PCa. A diagnosis of a more aggressive PCa may be treated using surgery, such as, but not limited to, radical prostatectomy, if such a surgery is deemed acceptable. In other embodiments, a more aggressive PCa may be treated using a therapeutic or pharmaceutical agent that targets PCAT18 and/or one or more related variants, and inhibits or silences the expression of PCAT18, as described below. After any of the above treatments, monitoring of the PCa is performed by periodically assessing the expression levels of PCAT18 and/or any related variants in a patient's biological sample, such as blood, plasma, urine or prostate tissue.

### Methods of Treating Prostate Cancer

Because PCAT18 has been found to be overexpressed in PCa, PCAT18 and related variants may also be used as a therapeutic target in PCa. Therefore, the present invention also relates to targeting PCAT18, including but not limited to additional transcript variants of PCAT18, modifications of PCAT18 and related variants, and other lncRNAs that may be transcribed from genomic locus LOC728606, using an inhibiting agent or therapeutic targeting strategy, such as antisense oligonucleotides, RNA interference (RNAi), esiRNA, shRNA, miRNA, decoys, RNA aptamers, small molecule inhibitors, RNA/DNA-binding proteins/peptides or other compounds with different formulations to inhibit one or more physiological actions effected by PCAT18 and to thereby treat PCa. Such therapeutic targeting strategies may be used to develop a therapeutic agent or pharmaceutical compositions that target PCAT18 and/or one or more related variants for treating PCa. Treatment of PCa may include administering to a subject having PCa a therapeutically effective amount of a therapeutic agent, such as an inhibiting agent of PCAT18 or a pharmaceutical composition, as described herein.

Accordingly, in a general aspect, the present invention provides for methods of treating a subject diagnosed with PCa by administering a therapeutically effective amount of an inhibiting agent of PCAT18. The inhibiting agent of PCAT18 may be an antisense oligonucleotide, RNA interference (RNAi), siRNA, esiRNA, shRNA, miRNA, decoys, RNA aptamers, small molecule inhibitors, RNA/DNA-binding proteins/peptides, or a combination thereof.

siRNA Molecules

In certain embodiments, the present invention provides for methods of treating PCa in a subject diagnosed with PCa using small interfering RNA (siRNA) molecules against PCAT18. siRNA molecules targeted to PCAT18 have been found to decrease proliferation of cancer cells when used as a single agent. For example, siRNA1, which comprises a nucleotide sequence corresponding to SEQ ID NO:22, and siRNA2, which comprises a nucleotide sequence corresponding to SEQ ID NO:23, both independently silence PCAT18. Moreover, siRNA1 and siRNA2 silencing of PCAT18 elicited significant and stable growth inhibition in a human prostate cancer cell line (C4-2) (see FIGS. 3(D) and 3(E)).

Generally, siRNAs used in the present invention are targeted to a PCAT18 gene, or the genomic region at locus LOC728606, and are capable of silencing or inhibiting the expression of PCAT18 RNA. siRNAs targeted to a PCAT18 gene or locus LOC728606 comprise a specific antisense sequence that is complementary to a portion of the noncoding RNA transcribed from the target gene (i.e., the target RNA) and can be double-stranded (i.e. composed of an antisense strand, comprising the specific antisense sequence, and a complementary sense strand) or single-stranded (i.e. composed of an antisense strand, comprising the specific antisense sequence, only) as described in more detail below. Short-hairpin siRNA (shRNA) against PCAT18 are also included in the present invention.

As is known in the art, the specificity of siRNA molecules is determined by the binding of the antisense strand of the molecule to its target RNA. Effective siRNA molecules are generally from 14 to 100 base pairs in length, or any length therebewteen to prevent them from triggering non-specific RNA interference pathways in the cell via the interferon response, although longer siRNA can also be effective. For example, the siRNA molecules contemplated by the present invention may be 14, 15, 16, 17, 18, 19, 20, 21, 22, 23, 24, 25, 30, 35, 40, 50, 60, 70, 80, 90, 100 base pairs in length or any number of base pairs therebetween in length.

Design and construction of siRNA molecules is known in the art (see, for example, Elbashir, et al., Nature, 411:494-498 (2001); Bitko and Barik, BMC Microbiol., 1:34 (2001)].

For the siRNA molecules used in the present invention, the target RNA is a noncoding RNA transcribed from the PCAT18 gene or the genomic region at locus LOC728606, including, without limitation, the nucleotide sequence corresponding to SEQ ID NO:1 (shown in FIG. 5). Therefore, in an embodiment, the target RNA for the PCAT18 siRNA is PCAT18 RNA corresponding to the nucleotide sequence as set forth in SEQ ID NO:1. The siRNA may comprise a sequence that is complementary to a target sequence within SEQ ID NO:1.

Suitable target sequences within the target RNA are selected using one or more of several criteria known in the art (see for example, Elbashir. S. M., et al. (2001) Nature 411, 494-498; Elbashir, S. M., et al. (2002) Methods 26, 199-213; Elbashir, S. M., et al. (2001) Genes Dev. 15, 188-200; Elbashir, S. M., et al. (2001) EMBO J. 20, 6877-6888; and Zamore, P. D., et al. (2000) Cell 101, 25-33). Target RNA sequences within the target RNA are typically between about 14 and about 50 nucleotides in length, or any length therebewteen, but may be longer in length, for example, the target RNA sequence may be about 60, 70, 80, 90, 100, 110, 120, 130, 140, 150, 160, 170, 180, 190, 200, 210, 220, 230, 240, 250 base pairs in length, or any number of base pairs therebetween in length. The target RNA sequence can be selected from various regions within the PCAT18 RNA. For example, siRNA1 comprises an antisense sequence SEQ ID NO:22 which is complementary to a target sequence within SEQ ID NO:1, and siRNA2 comprises an antisense sequence SEQ ID NO:23, which is complementary to a target sequence within SEQ ID NO:1.

Following selection of an appropriate target RNA sequence, as described above, siRNA molecules that comprise a nucleotide sequence complementary to all or a portion of the target RNA sequence, i.e. an antisense sequence, can be designed and prepared. As indicated above, the siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

Double-stranded siRNA may comprise RNA strands that are the same length or different lengths. In one embodiment, the siRNA is a double-stranded siRNA. In another embodiment, the siRNA is a double-stranded siRNA wherein both RNA strands are the same length.

Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNA interference.

Small hairpin RNA (shRNA) molecules thus are also contemplated by the present invention. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or the 5′ end of either or both strands). The spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). The spacer sequence is typically an unrelated nucleotide sequence that is situated between two complementary nucleotide sequence regions which, when annealed into a double-stranded nucleic acid, comprise a shRNA (see, for example, Brummelkamp et al., 2002 Science 296:550; Paddison et al., 2002 Genes Develop. 16:948; Paul et al., Nat Biotechnol 20:505-508 (2002); Grabarek et al., BioTechniques 34:734-44 (2003)). The spacer sequence generally comprises between about 3 and about 100 nucleotides.

Single-stranded siRNA molecules are generally single-stranded RNA molecules with little or no secondary structure.

The overall length of the siRNA molecules can vary from about 14 to about 100 nucleotides depending on the type of siRNA molecule being designed, and can be more than 100 nucleotides, such as, for example, about 14, 15, 16, 17, 18, 19, 20, 25, 30, 35, 40, 45, 50, 60, 70, 80, 90 100 nucleotides in length, or any number of nucleotides in length therebetween. For example, the siRNAs may be siRNA1 and siRNA2, as described above, corresponding to SEQ ID NO: 22 and SEQ ID NO: 23, respectively, which are each 36 oligonucleotides in length.

In an alternative embodiment, the siRNA molecule is a shRNA molecule or circular siRNA molecule between about 35 and about 100 nucleotides in length. In a further embodiment, the siRNA molecule is a shRNA molecule between about 40 to about 60 nucleotides in length.

As indicated above, the siRNA molecule comprises an antisense strand that includes a specific antisense sequence complementary to all or a portion of a target RNA sequence, such as, the PCAT18 noncoding RNA. One skilled in the art will appreciate that the entire length of the antisense strand comprised by the siRNA molecule does not need to be complementary to the target sequence. Thus, the antisense strand of the siRNA molecules may comprise a specific antisense sequence together with nucleotide sequences at the 5′ and/or 3′ termini that are not complementary to the target sequence. Such non-complementary nucleotides may provide additional functionality to the siRNA molecule. For example, they may provide a restriction enzyme recognition sequence or a “tag” that facilitates detection, isolation or purification. Alternatively, the additional nucleotides may provide a self-complementary sequence that allows the siRNA to adopt a hairpin configuration. Such configurations are useful when the siRNA molecule is a shRNA molecule, as described above.

The specific antisense sequence comprised by the siRNA molecule can be identical or substantially identical to the complement of the target RNA sequence. In the context of the present invention, the specific antisense sequence comprised by the siRNA molecule can be identical or substantially identical to the complement of the PCAT18 RNA sequence, that is, the complement of a contiguous portion of the RNA sequence corresponding to SEQ ID NO:1. In one embodiment of the present invention, the specific antisense sequence comprised by the siRNA molecule is at least about 75% identical to the complement of a contiguous portion of the RNA sequence corresponding to SEQ ID NO:1. In another embodiment, the specific antisense sequence comprised by the siRNA molecule is at least about 90% identical to the complement of a contiguous portion of the RNA sequence corresponding to SEQ ID NO:1. In a further embodiment, the specific antisense sequence comprised by the siRNA molecule is at least about 95% identical to the complement of a contiguous portion of the RNA sequence corresponding to SEQ ID NO:1. In another embodiment, the specific antisense sequence comprised by the siRNA molecule is at least about 98% identical to the complement of a contiguous portion of the RNA sequence corresponding to SEQ ID NO:1. Methods of determining sequence identity are known in the art and can be determined, for example, by using the BLASTN program of the University of Wisconsin Computer Group (GCG) software or provided on the NCBI website.

In one embodiment of the invention, the siRNA molecules comprise a specific antisense sequence that is capable of selectively hybridizing under stringent conditions to a portion of a naturally occurring target RNA, such as PCAT18 RNA. Suitable stringent conditions include, for example, hybridization according to conventional hybridization procedures and washing conditions of 1-3×SSC, 0.1-1% SDS, 50-700 C with a change of wash solution after about 5-30 minutes. As known to those of ordinary skill in the art, variations in stringency of hybridization conditions may be achieved by altering the time, temperature, and/or concentration of the solutions used for the hybridization and wash steps. Suitable conditions can also depend in part on the particular nucleotide sequences used, for example the portion of RNA sequence corresponding to SEQ ID NO:1.

The siRNA molecules can be prepared using several methods known in the art, such as chemical synthesis, in vitro transcription, the use of siRNA expression vectors, and any other conventional techniques known in the art. For example, general methods of RNA synthesis and use of appropriate protecting groups is well known in the art (see, for example, Scaringe, S. A., et al., J. Am. Chem. Soc, 1998, 120, 11820-11821; Matteucci, M. D. and Caruthers, M. H., J. Am. Chem. Soc, 1981, 103, 3185-3191; Beaucage, S. L. and Caruthers, M. H., Tetrahedron Left., 1981, 22, 1859-1862: Dahl, B. J., et al., Acta Chem. Scand., 1990, 44, 639-641; Reddy, M. P., et al., Tetrahedron Left, 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331). As is also well known in the art, modified siRNA molecules, such as phosphorothioated and alkylated derivatives, can also be readily prepared by similar methods

Various methods of testing the efficacy of the siRNA molecules are known in the art and may be employed to test the efficacy of the PCAT18 siRNA molecules, including siRNA1 and siRNA2.

Antisense Oligonucleotdies

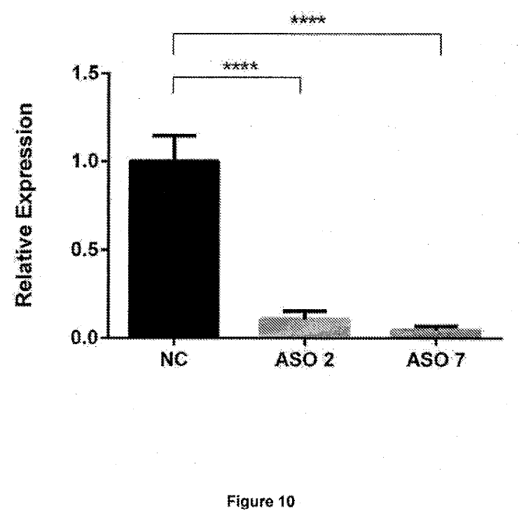

The present invention also provides for methods of treating PCa in a subject diagnosed with PCa using antisense oligonucleotides (ASOs). ASOs targeted to PCAT18 have been found to decrease proliferation of cancer cells when used as a single agent. For example, ASO2, which comprises a nucleotide sequence corresponding to SEQ ID No:24, and ASO7, which comprises a nucleotide sequence corresponding to SEQ ID NO:25 both independently silence PCAT18. Moreover, ASO2 and ASO7 inhibition of PCAT18 elicited significant knockdown of PCAT18 expression in a human prostate cancer cell line (C4-2) as compared to an antisense nucleotide (NC) with no known specific target in human or mouse genome (see FIG. 10).

Generally, ASOs used in the present invention are targeted to PCAT18 RNA, or any other additional RNA transcribed from the genomic region at locus LOC728606. The ASOs of the present invention are effective in reducing the amount of expression of PCAT18 RNA in vivo. ASOs targeted to the PCAT18 RNA or other transcripts derived from locus LOC728606 comprise a specific antisense sequence that is complementary to a portion of the noncoding RNA transcribed from the target gene (i.e., the target RNA) and can be either DNA, RNA or a chemical analogue. ASOs are generally single-stranded (i.e. composed of an antisense strand, comprising the specific antisense sequence, only) and bind to the complementary portion of the target RNA.

Suitable ASOs have a length of from about 12 to about 35 oligonucleotides and any amount therebewteen, and have sequence specificity (i.e., are complementary) to the PCAT18 noncoding RNA sequence. However, the ASOs of the present invention may compose more than about 35 oligonucleotides, for example, about 40, 50, 60, 70, 80, 90, 100, 110, 120, 130, 140, 150, 160, 170, 180, 190, 200, 210, 220, 230, 240, 250 oligonucleotides in length, or any number of oligonucleotides therebetween. Exemplary ASOs comprise a nucleotide sequence complementary to a contiguous portion of the nucleotide sequence (i.e. a target sequence) corresponding to SEQ ID NO:1. The contiguous portion of the nucleotide sequence may be between about 12 to about 250 oligonucleotides in length, or any number of oligonucleotides in length therebetween. For example, the ASOs may be ASO2 and ASO7, as described above, corresponding to SEQ ID NO: 24 and SEQ ID NO: 25, respectively, which are each 20 oligonucleotides in length.

The specific antisense sequence comprised by an ASO of the present invention can be identical or substantially identical to the complement of the target RNA sequence. In the context of the present invention, the specific antisense sequence comprised by the ASO molecule can be identical or substantially identical to the complement of the PCAT18 RNA sequence, that is, the complement of a contiguous portion of the RNA sequence corresponding to SEQ ID NO:1. In one embodiment of the present invention, the specific antisense sequence comprised by the ASO molecule is at least about 75% identical to the complement of a contiguous portion of the RNA sequence corresponding to SEQ ID NO:1. In another embodiment, the specific antisense sequence comprised by the ASO molecule is at least about 90% identical to the complement of a contiguous portion of the RNA sequence corresponding to SEQ ID NO:1. In a further embodiment, the specific antisense sequence comprised by the ASO molecule is at least about 95% identical to the complement of a contiguous portion of the RNA sequence corresponding to SEQ 10 NO:1. In another embodiment, the specific antisense sequence comprised by the ASO molecule s at least about 98% identical to the complement of a contiguous portion of the RNA sequence corresponding to SEQ ID NO:1. Methods of determining sequence identity are known in the art and can be determined, for example, by using the BLASTN program of the University of Wisconsin Computer Group (GCG) software or provided on the NCBI website.

In one embodiment of the invention, the ASO molecules comprise a specific antisense sequence that is capable of selectively hybridizing under stringent conditions to a portion of a naturally occurring target RNA, such as PCAT18 RNA or any other RNA transcribed from the genomic region at locus LOC728606 Suitable stringent conditions include, for example, hybridization according to conventional hybridization procedures and washing conditions of 1-3×SSC, 0.1-1% SDS, 50-7000 with a change of wash solution after about 5-30 minutes. As known to those of ordinary skill in the art, variations in stringency of hybridization conditions may be achieved by altering the time, temperature, and/or concentration of the solutions used for the hybridization and wash steps. Suitable conditions can also depend in part on the particular nucleotide sequences used, for example the portion of the antisense sequence corresponding to SEQ ID NO: 1

The oligonucleotides employed as ASOs in the present invention may be modified to increase the stability of the ASOs in vivo. For example, the ASOs may be employed as phosphorothioate derivatives (replacement of a non-bridging phosphoryl oxygen atoms with a sulfur atom) which have increased resistance to nuclease digestion (as done with ASO2 and ASO7). MOE modification (ISIS backbone) is also effective.

The ASOs used in the present invention may be prepared according to any of the methods that are well known to those of ordinary skill in the art. For example, the ASOs may be prepared by solid phase synthesis. See, Goodchild, J., Bioconjugate Chemistry, 1:165-167 (1990), for a review of the chemical synthesis of oligonucleotides. Alternatively, the ASOs can be obtained from a number of companies which specialize in the custom synthesis of oligonucleotides.

Administration of the Therapeutic Agents

Administration of the therapeutic agents described herein can be carried out using the various mechanisms known in the art, including naked administration and administration in pharmaceutically acceptable lipid carriers. For example, lipid carriers for ASO delivery are disclosed in U.S. Pat. Nos. 5,855,911 and 5,417,978 which are incorporated herein by reference. The carrier may also be any one of a number of sterols including cholesterol, cholate and deoxycholic acid. In general, the therapeutic agents describe herein, including the ASOs and siRNA molecules, may be administered by intravenous, intraperitoneal, subcutaneous or oral routes, or direct local tumor injection.

Suitable formulations for parenteral administration include aqueous solutions of the therapeutic agents in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers.

In other embodiments, a therapeutic agent may be co-administered with an agent which enhances the uptake of the therapeutic agent by the cells. For example, a therapeutic agent may be combined with a lipophilic cationic compound which may be in the form of liposomes. The use of liposomes to introduce nucleotides into cells is taught, for example, in U.S. Pat. Nos. 4,897,355 and 4,394,448, the disclosures of which are incorporated by reference in their entirety. See also U.S. Pat. Nos. 4,235,871, 4,231,877, 4,224,179, 4,753,788, 4,673,567, 4,247,411, 4,814,270 for general methods of preparing liposomes comprising biological materials.

In addition, the therapeutic agents described herein may be conjugated to a peptide that is ingested by cells. Examples of useful peptides include peptide hormones, antigens or antibodies, and peptide toxins. By choosing a peptide that is selectively taken up by the cancerous prostate cells, specific delivery of the therapeutic agent may be effected.

The amount of a therapeutic agent administered in the present methods describe herein is one effective to reduce the amount of PCAT18 expression. It will be appreciated that this amount will vary both with the effectiveness of the ASO, siRNA or other therapeutic inhibiting agent employed, and with the nature of any carrier used. The determination of appropriate amounts for any given therapeutic agent is within the skill in the art, through standard series of tests designed to assess appropriate therapeutic levels.

The therapeutic agents described herein may also be administered as part of a pharmaceutical composition or preparation containing suitable pharmaceutically acceptable carriers comprising excipients and auxiliaries which facilitate processing of the therapeutic agents into preparations which can be used pharmaceutically.

Accordingly, the present invention contemplates pharmaceutical compositions comprising a therapeutic agent effective to reduce the amount of PCAT18 in cancerous prostate cells exposed to the therapeutic agent, and a pharmaceutically acceptable carrier. The therapeutic agent may be an inhibiting agent of PCAT18, such as, for example, antisense oligonucleotides, RNA interference (RNAi), esiRNA, shRNA, miRNA, decoys, RNA aptamers, small molecule inhibitors, RNA/DNA-binding proteins/peptides or other compounds which inhibit the expression of PCAT18. In certain embodiments, the pharmaceutical composition may comprise one or more than one therapeutic agent, and a pharmaceutically acceptable carrier.

The pharmaceutical compositions used in the present invention include all compositions wherein the one or more than one therapeutic agent is contained in an amount which is effective to achieve inhibition of expression of PCAT18. While individual needs vary, determination of optimal ranges of effective amounts of each component is within the skill of the art.

The present invention further contemplates a method of treating PCa in a so subject comprising the administration of a therapeutically effective amount of a PCAT18 siRNA in combination with any other treatment, agent, drug, regimen or therapy, including without limitation, administration of ASOs, hormonal therapy, surgery, radiation therapy, chemotherapy, biologic therapy, bisphosphonate therapy, cryosurgery, high-intensity focused ultrasound, and proton beam radiation therapy. Alternatively, a method of treating PCa in a subject diagnosed with PCa may comprise administering a therapeutically effective amount of an ASO in combination with any other treatment or therapy, including without limitation, administration of PCAT18 siRNA molecules, hormonal therapy, surgery, radiation therapy, chemotherapy, biologic therapy, bisphosphonate therapy, cryosurgery, high-intensity focused ultrasound, and proton beam radiation therapy.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Patients and Methods

Patient-Derived Prostate Cancer Xenografts

PCa biopsy specimens were collected at the BC Cancer Agency with the patient's written informed consent. The protocol for this procedure was approved by the University of British Columbia (UBC) Research Ethics Board (REB). NOD/SCID mice used for this study were bred and maintained at the British Columbia Cancer Research Centre Animal Facility (Vancouver, Canada). All experimental protocols were approved by the University of British Columbia Animal Care Committee. Transplantable PCa tissue xenograft lines were established and maintained using subrenal capsule grafting as previously described (10).

LTL313B and LTL313H tumor tissue cell lines were derived from 2 primary neoplasm biopsies obtained simultaneously from the same patient (total biopsies performed=8). At the time of biopsy, the donor was affected by treatment-naive prostate adenocarcinoma (Gleason Score=8) with signs of pelvic infiltration and bone metastasis. Immediately after pathological diagnosis, the patient received hormonal therapy, and 9 months after commencing treatment PSA reached a nadir 0.28 ng/ml from a pre-treatment value of 19 ng/ml.

RNA Sequencing

Total RNA was extracted from non-metastatic LTL-313B and metastatic LTL-313H xenografts using RNAeasy kit (Qiagen), harvested on the same day, using Trizol (Invitrogen). RNA was sent to Otogenetics (Norcross, Ga.) for RNA sequencing. Sequenced reads were aligned to the hg19 human genome assembly and contrasted to the transcriptome generated from all the spliced sequences annotated in the RefSeq database using the DNAnexus suite (www.dnanexus.com). Transcript level was quantified by calculating the RPKM (reads per kilobase of transcript per million mapped reads) value (11). RPKM values were normalized to the root mean square (RMS) for each sample. Mapped transcripts were annotated using the gene cards database (www.genecards.org). Genes were categorized as “protein coding” and “non-coding” based on their functional annotation. Among non-coding sequences rRNAs, tRNAs, miRNAs snoRNAs and other known classes of RNAs were excluded from further analysis. LncRNAs were defined as all non-coding sequences longer than 200 bp and not belonging to other RNA categories. Based on those filtering criteria, 1653 lncRNAs expressed in PCa xenografts were identified.

Database Analysis

LOC728606 expression was also queried in Oncomine (www.oncomine.com) GEO (www.ncbi.nlm.nih.gov/geo/) and Cbio portal (www.cbioportal.org) gene expression databases. Analysis was restricted to PCa and prostate-derived samples.

Selected non-coding RNAs were analyzed through the cBio cancer genomic portal (12), which includes clinico-pathological and gene expression information from 29 normal prostate and 131 primary PCa samples (13). Gene expression data were downloaded from the portal as log 2 whole transcript normalized RNA expression values (Affymetrix Human Exon 1.0 ST arrays). To further characterize LOC728606 (JUPITER), expression patterns of JUPITER were analyzed in Oncomine (www.oncomine.com) and Gene Expression Omnibus (GEO) (http://www.ncbi.nim.nih.gov/geo/) databases, which include large collections of microarray data from human samples. Albeit these classical microarray platforms are restricted to mRNA detection, some of them might fortuitously hold probes matching a few lncRNAs. For Oncomine, a p value threshold of 0.01 (fold change>2) was selected. For GEO data (HG-U95D), normalized values were plotted and analyzed using Graph Pad Prism 6 software (La Jolla, Calif.).

Significance Analysis of Microarrays (SAM) was performed in R using the 20 PCa samples expressing the highest and lowest levels of PCAT18 from the Cbio database (21)prostate cancer samples (22). Transcripts positively associated with PCAT18 (with Q<0.5%) were analyzed using Oncomine to investigate correlations with clinical variables (threshold: p<E⁻⁴, odds ratio >2).

Clinical Samples

Characteristics of al enrolled patients are summarized in Table 1 below.

Prostate Tissue Samples:

Samples from patients with benign prostatic hyperplasia (BPH) or PCa were collected at the Stephanshorn Clinic in St. Gallen Switzerland, after study protocol approval by the local ethical committee. Resected specimens were immediately transferred on ice to the Institute for Pathology of the Kantons Hospital, St. Gallen for examination. Small tissue samples from macroscopically visible tumor and non-tumor prostate tissue were dissected, snap frozen in liquid nitrogen and cryo-preserved at −80° C. These samples were cut in a cryo-microtome and a slide of each probe was stained with hematoxylin-eosin for histological verification. RNA was isolated from frozen materials using the TRI-reagent (Ambion) method according to the manufacturer's guidelines. Extracted RNA was quality-checked using the Agilent Bioanalyzer 2100 (Agilent). The cDNA was synthesized from 1 μg of total RNA using Superscript II RNase H-reverse transcriptase (Invitrogen).

Plasma Samples:

Upon study protocol approval by UBC REB, and after obtaining written informed consent from study participants, blood samples and clinico-pathological data were collected at the British Columbia Cancer Agency (BCCA), Vancouver Centre. Three cohorts were evaluated: 25 individuals with no clinical sign of neoplasm; 25 PCa patients with treatment-naïve localized disease (Localized PCa); 25 patients with a clinically confirmed metastatic PCa and a progressive disease despite castration therapy (mCRPC). Samples were processed as previously described (4) for plasma separation, RNA extraction and retrotransciption.

TABLE 1
Clinical-pathological characteristics of enrolled patients.
PROSTATE TISSUE SAMPLES (N = 16)

| | | | Benign Prostatic | |
|---|---|---|---|---|
| | Prostate cancer | | Hyperplasia | |
| Median Age (Range) | 63 | (58-75) | 62 | (52-67) |
| Median PSA at | 8 | (4.5-23.1) ng/ml | 4.1 | (2.9-5.3) ng/ml |
| diagnosis (Range) | | | | |

| | | |
|---|---|---|
| Gleason Score | | |
| ≦8 | 5 | N.A. |
| ≧8 | 6 | N.A. |
| TNM Stage | | |
| T2a/N0/M0 | 2 | N.A. |
| T2c/N0/M0 | 5 | N.A. |
| T3/N0/M0 | 4 | N.A. |

PLASMA SAMPLES (N = 50)

| | Localized | | | |
|---|---|---|---|---|
| | Prostate Cancer | | mCRPC | |
| Median Age (Range) | 67 | (51-82) | 73 | (45-86) |
| Median PSA at | 9.2 | (0.6-22) ng/ml | 120 | (6.3-4948.5) ng/ml |
| diagnosis (Range) | | | | |

| | | |
|---|---|---|
| Risk Group | | |
| Intermediate | 22 | N.A. |
| Low | 3 | N.A. |
| High | | |
| Metastatic Sites | | |
| Bone | N.A. | 17 |
| Lymph nodes and | N.A. | 5 |
| Others | | |
| Others | N.A. | 3 |

“Risk groups” in Table 1 are defined based on pre-prostatectomy serum PSA value, T stage and Gleason Grade, as recommended by the Genito-Urinary Radiation Oncologists of Canada (2). PCa diagnosis was confirmed by pathological examination of tumor biopsies for each enrolled patient. Localized PCa cases were defined as those with no pathological evidence of lymph node dissemination and no clinical evidence of metastatic diffusion. PSA measurement and RNA extraction were performed on samples collected before prostatectomy and on treatment-naive patients. Metastatic cases were defined as those having clinical or pathological evidence of cancer dissemination to any of the following: lymph nodes, bones or soft tissues (lung, brain, spine, testis).

Quantitative PCR

Primers targeting selected lncRNAs were designed using BLAST software. Primer sequences (as listed in Table 2 below) were contrasted to the Homo Sapiens and Mus Musculus trancriptome to ensure their specificity for the intended target gene. Custom DNA oligos were provided by Invitrogen. RNA, extracted from xenografts as described above, was retrotranscribed using QuantiTect Reverse Transcription kit (Qiagen) following manufacturer's instructions. RNA extraction and retrotranscription for clinical samples are described above.

TABLE 2
Primer Sequences for Amplifying Select IncRNAs

| | SEQ ID | | SEQ ID | |
|---|---|---|---|---|
| Gene | NO. | Forward Primer | NO. | Reverse Primer |
| HPRT1 | 2 | GGTCAGGCAGTATAATCCAAAG | 3 | CGATGTCAATAGGACTCCAGATG |
| GAPDH | 4 | CACCAGGGCTGCTTTTAACTC | 5 | GACAAGCTTCCCGTTCTCAG |
| PCAT18 | 6 | AGGAGACAGGCCCCAGATTT | 7 | TGAAGTGCTGGGACAACGTA |
| PCGEM1 | 8 | TTGCCCTATGCCGTAACCTG | 9 | ACGTTGAGTCCCAGTGCATC |
| H19 | 10 | CCAGTGAGGAGTGTGGAGTAG | 11 | CAGCTGCCACGTCCTGTAAC |
| Linc461_1 | 12 | AGGAAACAGCTCTGGCATCC | 13 | CAGATTCCCCACCCCCTTTC |
| Linc461_3 | 14 | GTTCCTGCCCAGCTGGATTT | 15 | TCAGAGTAGTCCACGCCAGA |
| LOC285419 | 16 | TGACTCAACTTCTGGTGCAGAT | 17 | GGATGTGGCATATCTCTTGGTTTA |
| NCRNA116 | 18 | GAGACTGCTCAGAGGAAGAGAA | 19 | CAGACAGCCCAGTGTCTTGG |
| KLK3 | 20 | AGTGCGAGAAGCATTCCCAAC | 21 | CCAGCAAGATCACGCTTTTGTT |

Quantitative PCR was performed as previously described (14) using cDNA, primers and KAPA SYBR fast Universal Master Mix through ABIPrism 7900HT (Applied Biosystems) and following manufacturers' instructions. The 2^{-ΔΔCT} method was used for calculating the fold changes relative to the endogenous controls (HPRT and GAPDH, whose expression was stable in primary and metastatic xenografts, according to RNA Seq. data). Primers specific for the 2 main variants of linc461 (transcript variant 1 LINC461_1 and transcript variant 3 LINC461_3) were designed to confirm separately their up-regulation. For plasma sample studies, mir30e was used for normalization since its expression has been shown to be stable in plasma samples of normal and PCa patients (4).

To confirm PCAT18 expression patterns with another methodology, Applied Biosystem Non-coding RNA assay Hs03669364_m1 was employed, which is specific for LOC728606 (PCAT18) and spans the exon1-exon2 boundary. QPCR was performed according to manufacturer's instructions on the ABIPrism 7900HT (Applied Biosystems). The 2^{-ΔΔT} method was used for calculating the fold changes relative to endogenous control (GAPDH).

TaqMan qPCR was also performed to quantify the sub-cellular localization of PCAT18. GAPDH and MALAT1 (Hs00273907_s1). Total, cytoplasmic and nuclear RNA was extracted and purified using the Ambion PARIS kit (Life Technologies), following manufacturer's instruction.

In Vitro Experiments

Unless otherwise specified, Prostate cancer- and benign prostatic hyperplasia-derived cell lines were maintained in 10% fetal bovine serum (GIBCO, Life Technologies) and RPMI 1640 growth medium (GIBCO, Life Technologies).

Gene Silencing: cells were treated with 2 nM PCAT18 (LOC728606)-targeting siRNAs (siRNA1 and siRNA2) or negative control (NC) reagent (Dicer substrate siRNAs, Integrated DNA Technology, Duplex names: NR_024259_1 (siRNA1); NR_024259_2 (siRNA2); DS_NC1), following manufacturer's instructions. NC (negative transfection control) is a DsiRNA duplex that does not target any known human or mouse transcript. Lipofectamine RNAiMaX (Invitrogen) was employed as the transfection reagent RNA extraction, retrotranscription and qPCR were performed as described in FIG. 1(B) legend.

MTT assay was performed on LNCaP, C4-2 and BPH cells treated with NC or PCAT18-targeting siRNAs (both at 2 nM concentration) on days 1-3-5 post-transfection, as previously described (Watahiki A, et al. MicroRNAs associated with metastatic prostate cancer. PloS one. 2011; 6(9):e24950).

Caspase 3 and 7 activity was quantified through Caspese-Glo 3/7 assay (Promega), as previously described (Crea F, et al. BMI1 silencing enhances docetaxel activity and impairs antioxidant response in prostate cancer. International journal of cancer Joumal international du cancer. 2011; 128(8):1946-1954) on cells transfected with the above described protocol.

The wound healing assay was performed in triplicate on C4-2 cells as previously described (Decker K F, et al. Persistent androgen receptor-mediated transcription in castration-resistant prostate cancer under androgen-deprived conditions. Nucleic acids research. 2012; 40(21):10765-10779). Transfection protocols were identical to those described above. 12 hours post-transfection, a ‘wound’ was produced using a P20 pipette tip. Pictures were taken at marked spots 0-6-24-48 h post-wounding, using a Zeiss Axiovert 40 CFL inverted microscope connected to Axiovision 4.7 software. Invasion assay was performed in triplicate on C4-2 cells using BD BioCoat™ BD Matrigel™ Invasion Chambers (24-well plates) and following manufacturers instructions. Transfection was performed on day 0, as described above. After 12 hours, cells were plated in the invasion chambers. 16 hours post-plating, we followed a previously described method for analysis and quantification of invading cells (Crea F, et al. Pharmacologic disruption of Polycomb Repressive Complex 2 inhibits tumorigenicity and tumor progression in prostate cancer. Molecular cancer. 2011; 10:40).

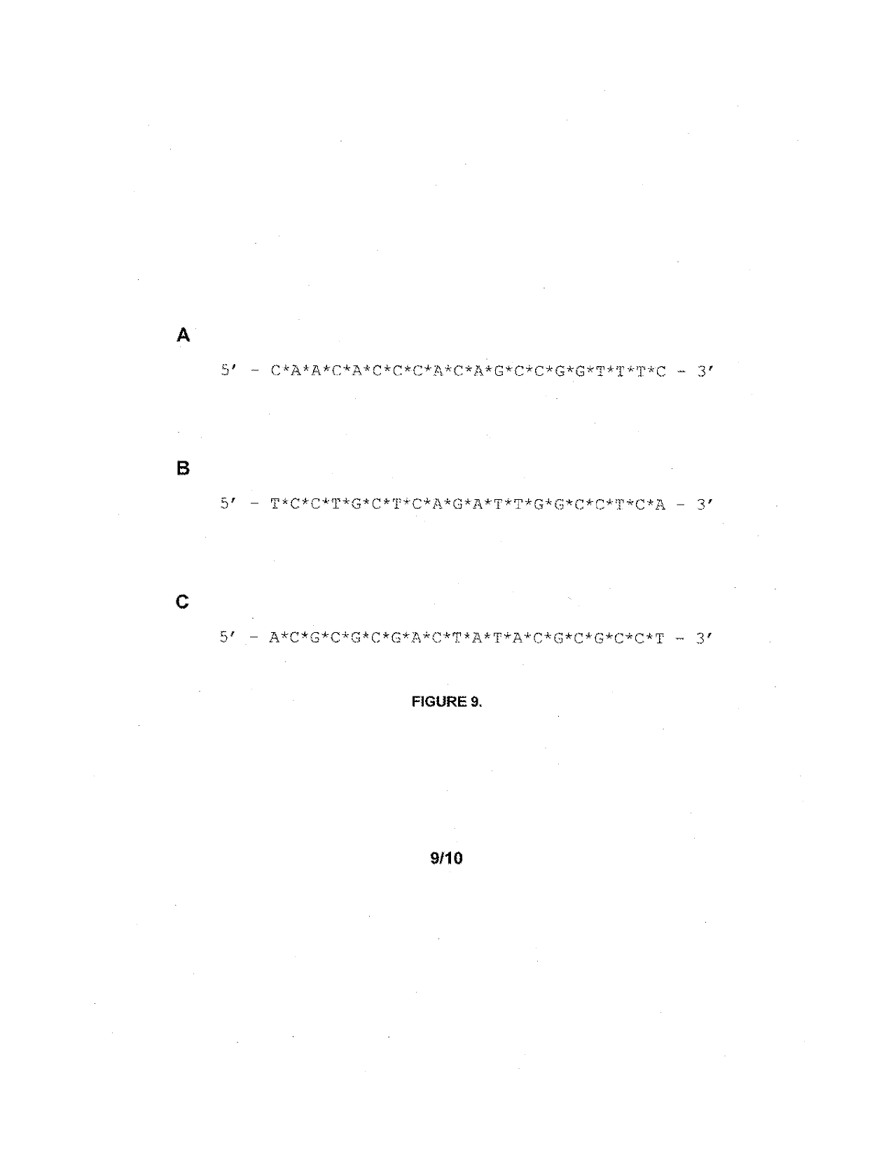

Antisense Oligonucleotide Knockdown. C4-2 cells were treated with 160 nM of PCAT18-targeting antisense oligonucleotides (ASO2 and ASO7), or antisense oligonucleotide (NC) with no known specific target in human or mouse genome. ASO sequences ASO2 and ASO7 are detailed in FIGS. 9(B) and 9(C), respectively. The antisense oligonucleotide (NC) is detailed in FIG. 9(A). The antisense oligonucleotides were purchased from Integrated DNA Technologies. The ‘*’ in the sequences represent phosphorothioate backbone. The oligonucleotides were re-suspended in 1×TE buffer as per manufacturers instructions. ASO transfections were performed following manufacturers instructions using Oligofectamine (Life Technologies) as the transfecting reagent. Gene quantification (via quantitative PCR) was performed as described above.

Statistical Analysis

Unless otherwise specified, all statistical analyses were performed using Graph Pad Prism 6 software (La Jolla, Calif.).

### Example 1: Identification of PCAT18

The identification of novel biomarkers and therapeutic targets for mCRPC has been hampered by the lack of suitable models that accurately reflect the clinical reality. This hurdle has been overcome by the generation of xenograft models developed from primary patient samples. In the present application, 2 PCa xenograft lines were exploited: LTL-313B and LTL-313H. Both models were derived from PCa biopsies of the same patient, yet they display a strikingly different phenotype. LTL-313B cells (non-metastatic) showed little local invasion and no distant metastasis while LTL-313H xenografts (metastatic) showed invasion into the mouse host kidney and distant metastases were detectable in the hosts' lungs 3 months after engraftment (FIG. 1A).

RNA Sequencing was performed on paired metastatic/non-metastatic PCa orthotopic xenografts derived from clinical specimens. The most differentially expressed lncRNA was further analyzed in clinical samples and publically available databases.

New lncRNAs associated with PCa metastasis were identified using RNA Sequencing analysis on the patient-derived PCa xenografts was performed using the strategy outlined in Table 3.

TABLE 3
RNA Sequence Analysis on Matched Primary and Metastatic PCa
Xenografts.

| | Non-metastatic | Metastatic |
|---|---|---|
| Mapped Reads | 17,259,797 | 15,244,256 |
| Unique Reads | 15,711,473 | 13,704,330 |
| Protein coding genes | 18872 | 18872 |
| Up-regulated IncRNAs (total) | 77 (1668) | 153 (1668) |

In Table 3 above, genes were categorized as “protein coding” and “non-coding” based on their functional annotation. Among non-coding sequences rRNAs, tRNAs, miRNAs and other known classes of RNAs were excluded from further analysis. LncRNAs were defined as all non-coding sequences longer than 200 bp and not belonging to other RNA categories. Based on those filtering criteria, 1668 lncRNAs expressed in PCa xenografts were identified.

Sequencing analysis on the patient-derived PCa xenografts showed that 153 lncRNAs were up-regulated and 77 were down-regulated in metastatic vs non-metastatic xenografts (see Tables 4 and 5 below). The vast majority of these transcripts have not been previously characterized. Of note, the list of up-regulated transcripts included two known oncogenic lncRNAs, H19 and PCGEM1 (8, 15), while the down-regulated transcripts included the only known onco-suppressive lncRNA in PCa (PTENP1) (16). PCA3 was detectable in both models, but its differential expression was below the significance threshold (LTL313H vs. LTL313B RPKM ratio=1.38). One or more than one of the lncRNAs listed in Table 4 may be used in combination with PCAT18 in order to diagnose or treat prostate cancer using the methods as described herein.

TABLE 4
IncRNAs Up-regulated in Metastatic (313H) vs. Localized (313B)
PCa xenografts.

| Gene Name | Gene Coordinates | Category | RPKM 313H | RPKM 313B |
|---|---|---|---|---|
| LOC728606 | chr18: 24267585-24283602 | RNA gene | 2.992 | 0.9714 |
| PCGEM1 | chr2: 193614571-193641621 | RNA gene | 2.71 | 0.3074 |
| LOC100329109 | chr2: 206980297-206981296 | pseudogene | 1.242 | 0.3546 |
| Linc461 | chr5: 87960264-87969146 | RNA gene | 0.8182 | 0.1868 |
| C21orf82 | chr21: 35552978-35562220 | RNA gene | 0.7301 | 0.1746 |
| FLJ40852 | chr7: 141404138-141438030 | RNA gene | 0.7177 | 0.2155 |
| C6orf41 | chr6: 26924772-26991752 | RNA gene | 0.6849 | 0.2271 |
| NCRNA00235 | chr16: 576847-577407 | RNA gene | 0.6809 | 0.237 |
| PMS2L4 | chr7: 66757424-66767406 | pseudogene | 0.665 | 0.2341 |
| NCRNA00092 | chr9: 98782016-98784037 | RNA gene | 0.6636 | 0.2569 |
| FLJ12825 | chr12: 54452038-54516018 | RNA gene | 0.6541 | 0.2586 |
| LOC286094 | chr8: 136246374-136311959 | RNA gene | 0.5404 | 0.1792 |
| PRO0628 | chr20: 39666473-39667632 | uncategorized | 0.5351 | 0.1911 |
| LOC441046 | chr4: 144480625-144482612 | pseudogene | 0.5284 | 0.2453 |
| RPL13AP3 | chr14: 56232963-56234434 | pseudogene | 0.519 | 0.1806 |
| LOC100009676 | chr3: 101395274-101398055 | RNA gene | 0.5134 | 0.1111 |
| LOC644145 | chr4: 56686237-56703430 | pseudogene | 0.4923 | 0.1142 |
| C2orf58 | chr2: 38358247-38408991 | RNA gene | 0.4909 | 0.2278 |
| GTF2IRD2P1 | chr7: 72656902-72685658 | pseudogene | 0.4833 | 0.01473 |
| ISCA1P1 | chr5: 62071202-62073170 | pseudogene | 0.4832 | 0.189 |
| LOC285419 | chr4: 124695419-124786732 | RNA gene | 0.4758 | 0.1405 |
| NCRNA00116 | chr2: 110969106-110980517 | RNA gene | 0.4719 | 0.1095 |
| ADAM6 | chr14: 106435819-106438358 | pseudogene | 0.4666 | 0.1925 |
| TRIM78P | chr11: 5664412-5687608 | pseudogene | 0.4537 | 0.07019 |
| TYRO3P | chr15: 76551630-76552493 | pseudogene | 0.4421 | 0.0513 |
| LOC100131193 | chr9: 139698379-139703300 | RNA gene | 0.4403 | 0.1486 |
| RPS10P7 | chr1: 201489032-201489718 | pseudogene | 0.4169 | 0.06443 |
| LOC339568 | chr20: 37842424-37853391 | RNA gene | 0.41 | 0.1464 |
| HAS2AS | chr8: 122651586-122656933 | RNA gene | 0.4079 | 0.05409 |
| C16orf67 | chr16: 31711934-31718743 | RNA gene | 0.4037 | 0.09994 |
| LOC440839 | chr2: 113917421-114205429 | pseudogene | 0.4014 | 0.187 |
| LOC154761 | chr7: 143509061-143533810 | RNA gene | 0.3968 | 0.1929 |
| HULC | chr6: 8652442-8654077 | RNA gene | 0.3963 | 0.09196 |
| SNHG12 | chr1: 28905052-28908366 | RNA gene | 0.3887 | 0.1804 |
| C2orf52 | chr2: 232373137-232379050 | RNA gene | 0.3725 | 0.1694 |
| H19 | chr11: 2016406-2019065 | RNA gene | 0.3724 | 0.03841 |
| UOX | chr1: 84830648-84863576 | pseudogene | 0.3711 | 0.1722 |
| DIRC3 | chr2: 218148748-218621316 | RNA gene | 0.3669 | 0.1476 |
| PCNAP1 | chr4: 100081753-100082804 | pseudogene | 0.3631 | 0.1264 |
| C1orf220 | chr1: 178511931-178518024 | RNA gene | 0.3515 | 0.086 |
| HTR7P1 | chr12: 13153376-13157762 | pseudogene | 0.3478 | 0.1716 |
| CTSLL2 | chr10: 48155943-48158691 | pseudogene | 0.3457 | 0.1203 |
| C6orf176 | chr6: 166337536-166401527 | RNA gene | 0.3402 | 0.07894 |
| LOC1518 | chr9: 90459660-90462338 | pseudogene | 0.3363 | 0.1561 |
| PIPSL | chr10: 95717897-95721672 | pseudogene | 0.3274 | 0.1408 |
| FLJ41941 | chr22: 18512151-18520734 | RNA gene | 0.3211 | 0.1489 |
| C21orf71 | chr21: 26955088-26955536 | RNA gene | 0.319 | 0.09872 |
| C8ORFK29 | chr8: 145576887-145578505 | RNA gene | 0.3159 | 0.04887 |
| LOC339535 | chr1: 238643686-238649317 | RNA gene | 0.3123 | 0.02899 |
| C21orf41 | chr21: 30968360-31003067 | RNA gene | 0.3115 | 0.08262 |
| MCM3APAS | chr21: 47649158-47671604 | RNA gene | 0.3095 | 0.1149 |
| LOC257358 | chr5: 169758435-169762104 | RNA gene | 0.3033 | 0.06033 |
| C1orf180 | chr1: 85093913-85100703 | RNA gene | 0.298 | 0.09222 |
| LOC646813 | chr11: 50368318-50379802 | pseudogene | 0.298 | 0.1037 |
| CIDECP | chr3: 10059237-10067820 | pseudogene | 0.2973 | 0.1104 |
| ZSCAN12P1 | chr6: 28058929-28063492 | pseudogene | 0.296 | 0.1314 |
| LOC389634 | chr12: 8509562-8543348 | RNA gene | 0.2898 | 0.1345 |
| C17orf91 | chr17: 1614799-1619566 | RNA gene | 0.2845 | 0.1337 |
| LOC282997 | chr10: 112628648-112630662 | RNA gene | 0.2844 | 0.06599 |
| LOC146880 | chr17: 62774258-62777622 | RNA gene | 0.2837 | 0.07714 |
| RBMY2FP | chrY: 24455006-24462350 | pseudogene | 0.2809 | 0.04346 |
| LOC152024 | chr3: 24141465-24144738 | RNA gene | 0.277 | 0.07911 |
| C3orf51 | chr3: 55691245-55693497 | RNA gene | 0.2755 | 0.01967 |
| C14orf132 | chr14: 96505662-96560133 | RNA gene | 0.2742 | 0.1238 |
| LOC100129396 | chr17: 16692057-16693815 | RNA gene | 0.2713 | 0.1008 |
| NCRNA00093 | chr10: 101686966-101718755 | RNA gene | 0.2701 | 0.1337 |
| LOC401010 | chr2: 132199734-132202467 | pseudogene | 0.262 | 0.0486 |
| LOC285692 | chr5: 9641428-9903936 | RNA gene | 0.2589 | 0.02404 |
| LOC100128842 | chr1: 1193438-1196954 | RNA gene | 0.2574 | 0.1008 |
| RPSAP52 | chr12: 66151803-66220754 | pseudogene | 0.2511 | 0.03885 |
| DUSP5P | chr1: 228780657-228788159 | pseudogene | 0.2511 | 0.07172 |
| FASAS | chr10: 90751183-90752732 | RNA gene | 0.2465 | 0.1144 |
| PIN1L | chr1: 70385005-70386000 | pseudogene | 0.2397 | 0.08901 |
| SUMO1P3 | chr1: 160287055-160288258 | pseudogene | 0.2379 | 0.07363 |
| TTC3L | chrX: 74960373-74962914 | pseudogene | 0.2254 | 0.06971 |
| NCRNA00110 | chr21: 31120494-31136323 | RNA gene | 0.2188 | 0.04063 |
| C14orf34 | chr14: 56247854-56263392 | RNA gene | 0.2142 | 0.06629 |
| NCRNA00052 | chr15: 88120160-88122917 | RNA gene | 0.2139 | 0.04964 |
| SOX2OT | chr3: 181328151-181459003 | RNA gene | 0.2107 | 0.07112 |
| FLJ14107 | chr8: 22497884-22499722 | RNA gene | 0.2077 | 0.07231 |
| FLJ13224 | chr12: 31477250-31478879 | RNA gene | 0.2051 | 0.02719 |
| NCRNA00174 | chr7: 65841032-65865395 | RNA gene | 0.2051 | 0.08016 |
| C6orf217 | chr6: 135818939-136011975 | RNA gene | 0.2044 | 0.08133 |
| LOC100130522 | chr18: 77905807-77929616 | RNA gene | 0.1957 | 0.05318 |
| LOC400752 | chr1: 45769582-45771290 | RNA gene | 0.1956 | 0.05187 |
| FAM92A3 | chr4: 183958818-183961271 | pseudogene | 0.1946 | 0.05419 |
| C3orf65 | chr3: 185431040-185435955 | RNA gene | 0.1945 | 0.04514 |
| TMPRSS8P | chr16: 2889575-2892752 | pseudogene | 0.1935 | 0.04489 |
| BTN2A3 | chr6: 26421619-26430816 | pseudogene | 0.1923 | 0.08926 |
| LOC100129534 | chr1: 2281853-2284100 | pseudogene | 0.1912 | 0.07887 |
| LOC255512 | chr11: 1330938-1331936 | RNA gene | 0.1912 | 0.08874 |
| EP400NL | chr12: 132568828-132610885 | pseudogene | 0.1862 | 0.0576 |
| AKR7L | chr1: 19592476-19600568 | pseudogene | 0.1804 | 0.06281 |
| BPESC1 | chr3: 138823027-138844003 | RNA gene | 0.1768 | 0.07573 |
| SIGLEC16 | chr19: 50472912-50479075 | pseudogene | 0.1763 | 0.05513 |
| NCRNA00176 | chr20: 62665697-62671314 | RNA gene | 0.1734 | 0.05082 |
| LOC100130015 | chr16: 90106171-90114033 | pseudogene | 0.1729 | 0.06581 |
| TTTY11 | chrY: 8651359-8685423 | RNA gene | 0.1728 | 0.05347 |
| MGC23270 | chr14: 105287538-105290055 | RNA gene | 0.1707 | 0.07041 |
| C9orf106 | chr9: 132083295-132084882 | RNA gene | 0.1699 | 0.05258 |
| C11orf64 | chr11: 60383224-60454619 | RNA gene | 0.1687 | 0.05221 |
| GSTM2P1 | chr6: 111367623-111368757 | pseudogene | 0.1683 | 0.03905 |
| NCRNA00203 | chr14: 93533797-93538476 | RNA gene | 0.1632 | 0.07577 |
| NCRNA00226 | chr14: 106744269-106744965 | RNA gene | 0.1608 | 0.07462 |
| FAM153C | chr5: 177435689-177474656 | pseudogene | 0.1585 | 0.0735 |
| LOC723809 | chr7: 104535075-104567092 | RNA gene | 0.1564 | 0.07258 |
| LOC653113 | chr12: 8383646-8395542 | pseudogene | 0.1548 | 0.06203 |
| MGC16275 | chr17: 72206141-72209460 | RNA gene | 0.1541 | 0.05364 |
| PRNT | chr20: 4711929-4721314 | RNA gene | 0.153 | 0.06347 |
| SH3GL1P1 | chr17: 30367355-30369851 | pseudogene | 0.1529 | 0.01773 |
| C4orf12 | chr4: 85887971-85928168 | RNA gene | 0.1522 | 0.0565 |
| LOC285456 | chr4: 109459346-109541613 | RNA gene | 0.1519 | 0.04699 |
| BTF3L1 | chr13: 77502585-77503223 | pseudogene | 0.1495 | 0.06916 |
| INTS4L1 | chr7: 64601603-64694599 | pseudogene | 0.1494 | 0.06938 |
| FLJ40504 | chr17: 26603012-26634408 | pseudogene | 0.1469 | 0.02273 |
| CTSL3 | chr9: 90387830-90401799 | pseudogene | 0.1454 | 0.06747 |
| TUBB4Q | chr4: 190903679-190906024 | pseudogene | 0.1453 | 0.03393 |
| CYP2D7P1 | chr22: 42536216-42540575 | pseudogene | 0.1422 | 0.05279 |
| C17orf44 | chr17: 8123949-8127361 | RNA gene | 0.1369 | 0.06353 |
| MORF4 | chr4: 174537088-174537794 | pseudogene | 0.1351 | 0.06269 |
| PYY2 | chr17: 26553589-26555083 | pseudogene | 0.1341 | 0.0415 |
| FLJ39534 | chr3: 47205860-47285605 | RNA gene | 0.1316 | 0.05237 |
| LOC650368 | chr11: 3402191-3430378 | pseudogene | 0.1293 | 0.03001 |
| LOC100133957 | chrX: 47518252-47519776 | RNA gene | 0.1252 | 0.05813 |
| HLA-DPB2 | chr6: 33080293-33096890 | pseudogene | 0.1186 | 0.05506 |
| C21orf15 | chr21: 15215455-15220685 | RNA gene | 0.1119 | 0.03464 |
| PPP1R2P9 | chrX: 42636619-42637486 | pseudogene | 0.11 | 0.05107 |
| FLJ40292 | chr9: 140657474-140659222 | RNA gene | 0.1092 | 0.02534 |
| CSDAP1 | chr16: 31579088-31580845 | pseudogene | 0.1086 | 0.05043 |
| LOC643387 | chr2: 239140327-239142983 | pseudogene | 0.1078 | 0.01668 |
| LOC642846 | chr12: 9436253-9466684 | RNA gene | 0.1075 | 0.02531 |
| TDH | chr8: 11197146-11225961 | pseudogene | 0.1056 | 0.03266 |
| NCRNA00087 | chrX: 134229015-134232733 | RNA gene | 0.102 | 0.04731 |
| PI4KAP1 | chr22: 20383731-20398695 | pseudogene | 0.1012 | 0.03756 |
| UBE2MP1 | chr16: 34403802-34404762 | pseudogene | 0.09938 | 0.04612 |
| SSX8 | chrX: 52651985-52662998 | pseudogene | 0.09907 | 0.03065 |
| NF1P1 | chr15: 21122021-21134625 | pseudogene | 0.09875 | 0.04582 |
| C21orf90 | chr21: 45937098-45938859 | RNA gene | 0.09845 | 0.0457 |
| LOC553137 | chr6: 107218007-107222877 | uncategorized | 0.09721 | 0.0361 |
| LOC154822 | chr7: 158801045-158818928 | RNA gene | 0.09521 | 0.04395 |
| C8orf51 | chr8: 144448794-144450805 | RNA gene | 0.09493 | 0.02203 |
| LOC285501 | chr4: 178649911-178911903 | RNA gene | 0.09245 | 0.04291 |
| TBC1D3P2 | chr17: 60342069-60353016 | pseudogene | 0.09224 | 0.04303 |
| DKFZp434L192 | chr7: 56563916-56564977 | RNA gene | 0.08992 | 0.04174 |
| LOC100189589 | chr2: 74612845-74621008 | RNA gene | 0.08909 | 0.04135 |
| FLJ36000 | chr17: 21904062-21913070 | RNA gene | 0.08801 | 0.03501 |
| FAM35B2 | chr10: 47379720-47421236 | pseudogene | 0.08689 | 0.04029 |
| LOC283761 | chr15: 90048162-90067265 | RNA gene | 0.0808 | 0.03771 |
| LOC149837 | chr20: 5479218-5485242 | RNA gene | 0.07978 | 0.02469 |
| LOC100134259 | chr2: 47055003-47086145 | RNA gene | 0.07893 | 0.03663 |
| LOC100288778 | chr12: 87984-91262 | pseudogene | 0.07816 | 0.03624 |
| C14orf48 | chr14: 94463642-94478040 | RNA gene | 0.0749 | 0.02369 |
| KIAA0125 | chr14: 106383838-106398500 | RNA gene | 0.07452 | 0.01845 |

Displayed genes showed an RMS-normalized RPKM ratio higher than 2 and were ranked based on expression level in 313H cells.

TABLE 5
IncRNAs Down-regulated in a metastatic (313H) vs. Localized
(313B) PCa xenograft.

| | | | RPKM | |
|---|---|---|---|---|
| Gene Name | Gene Coordinates | Category | 313H | RPKM 313B |
| BCL8 | chr15: 20874797-20961480 | pseudogene | 1.48 | 4.125 |
| RPL23AP32 | chr2: 54756359-54756978 | pseudogene | 1.494 | 3.507 |
| GNRHR2 | chr1: 145509820-145516076 | pseudogene | 0.9937 | 3.053 |
| LOC643837 | chr1: 763064-789740 | RNA gene | 0.4947 | 1.406 |
| RPL29P2 | chr17: 7657638-7658284 | pseudogene | 0.2952 | 0.8221 |
| C8orf56 | chr8: 104145192-104153570 | RNA gene | 0.2894 | 0.8059 |
| LOC146481 | chr16: 34711785-34714967 | pseudogene | 0.2624 | 0.6819 |
| LOC286467 | chrX: 130836679-130964671 | RNA gene | 0.1293 | 0.6511 |
| PAR1 | chr15: 25380789-25383200 | RNA gene | 0.2574 | 0.6432 |
| OR4N3P | chr15: 22413462-22414393 | pseudogene | 0.1025 | 0.6182 |
| BDNFOS | chr11: 27528399-27699348 | RNA gene | 0.2591 | 0.6119 |
| HPVC1 | chr7: 54268917-54270114 | RNA gene | 0.1993 | 0.592 |
| DLEU2L | chr1: 64014651-64016307 | pseudogene | 0.2305 | 0.5885 |
| LOC100130987 | chr11: 67085310-67159158 | RNA gene | 0.1685 | 0.5736 |
| SAA3P | chr11: 18134020-18137679 | pseudogene | 0.149 | 0.5532 |
| SBDSP1 | chr7: 72299952-72307976 | pseudogene | 0.1685 | 0.4984 |
| LOC153684 | chr5: 43042236-43045370 | RNA gene | 0.2113 | 0.4903 |
| LOC440944 | chr3: 9430537-9439174 | RNA gene | 0.154 | 0.4766 |
| RPL13AP6 | chr10: 112696361-112697013 | pseudogene | 0.1462 | 0.4752 |
| LOC284900 | chr22: 28315364-28398665 | RNA gene | 0.1402 | 0.4212 |
| LOC150568 | chr2: 105050805-105129214 | RNA gene | 0.1587 | 0.4126 |
| LOC202781 | chr7: 154795143-154797412 | RNA gene | 0.08414 | 0.41 |
| RPL32P3 | chr3: 129101678-129118282 | pseudogene | 0.1151 | 0.4058 |
| LOC121838 | chr13: 44596471-44604598 | RNA gene | 0.1304 | 0.3632 |
| LOC595101 | chr16: 30278914-30346695 | pseudogene | 0.111 | 0.3606 |
| RAB9P1 | chr5: 104435175-104435798 | pseudogene | 0.153 | 0.3552 |
| LOC100132215 | chr2: 63271100-63275656 | RNA gene | 0.05064 | 0.3525 |
| LOC202181 | chr5: 177045501-177099278 | RNA gene | 0.06685 | 0.3413 |
| LOC100233209 | chr12: 47602203-47610226 | RNA gene | 0.1336 | 0.3366 |
| LOC100101938 | chr13: 19836941-19919113 | pseudogene | 0.1282 | 0.3332 |
| LOC283914 | chr16: 34597902-34624953 | RNA gene | 0.1045 | 0.3233 |
| LOC80054 | chr19: 33793763-33795962 | RNA gene | 0.06511 | 0.3224 |
| SMAD5OS | chr5: 135465205-135470579 | RNA gene | 0.1321 | 0.3189 |
| LOC648691 | chr22: 22901756-22909006 | RNA gene | 0.06153 | 0.3141 |
| FLJ43390 | chr14: 62584075-62595131 | RNA gene | 0.1107 | 0.3082 |
| C18orf18 | chr18: 5236724-5238028 | RNA gene | 0.07318 | 0.3057 |
| LOC285733 | chr6: 131148324-131156430 | RNA gene | 0.1074 | 0.2992 |
| HEJ1 | chr1: 102337567-102360299 | pseudogene | 0.1072 | 0.2985 |
| LOC285735 | chr6: 133409219-133427710 | RNA gene | 0.1187 | 0.2938 |
| FLJ37307 | chr13: 52387483-52419286 | RNA gene | 0.04795 | 0.2893 |
| CCT6P1 | chr7: 65216092-65228661 | pseudogene | 0.1082 | 0.276 |
| LOC127841 | chr1: 204337558-204338847 | RNA gene | 0.111 | 0.2749 |
| FLJ35390 | chr7: 44079067-44082081 | RNA gene | 0.089 | 0.2685 |
| HSP90B3P | chr1: 92100568-92109334 | pseudogene | 0.07017 | 0.2605 |
| C1orf213 | chr1: 23695464-23698278 | RNA gene | 0.1018 | 0.2499 |
| LOC284788 | chr20: 22380971-22401281 | RNA gene | 0.08953 | 0.2493 |
| PTENP1 | chr9: 33673507-33677418 | pseudogene | 0.08543 | 0.2492 |
| TMEM191A | chr22: 21055402-21058891 | RNA gene | 0.07316 | 0.249 |
| MCART3P | chr6: 66497772-66499375 | pseudogene | 0.05954 | 0.2487 |
| LOC286359 | chr9: 100153121-100158973 | RNA gene | 0.09479 | 0.242 |
| LOC100379224 | chr19: 44609494-44617336 | RNA gene | 0.08527 | 0.2375 |
| LOC728723 | chr5: 76382623-76444175 | RNA gene | 0.0837 | 0.2331 |
| LOC541473 | chr7: 72440192-72443660 | pseudogene | 0.09825 | 0.2279 |
| SYT14L | chr4: 68926330-68929015 | pseudogene | 0.07273 | 0.2194 |
| GK3P | chr4: 166198944-166201175 | pseudogene | 0.0855 | 0.2183 |
| ST7OT3 | chr7: 116822735-116849991 | RNA gene | 0.09326 | 0.2164 |
| EGOT | chr3: 4790880-4793274 | RNA gene | 0.06541 | 0.2125 |
| ARMCX4 | chrX: 100673266-100788446 | RNA gene | 0.03532 | 0.1967 |
| AFG3L1 | chr16: 90038988-90063028 | pseudogene | 0.07533 | 0.1959 |
| SMCR5 | chr17: 17680000-17682843 | RNA gene | 0.06716 | 0.1868 |
| LOC100126784 | chr11: 19732481-19736146 | RNA gene | 0.0521 | 0.1865 |
| PMS2L11 | chr7: 76610139-76653074 | pseudogene | 0.05429 | 0.1763 |
| AMZ2P1 | chr17: 62962668-62971703 | RNA gene | 0.02775 | 0.1674 |
| MGC2889 | chr3: 192959568-192961760 | RNA gene | 0.06532 | 0.1617 |
| LOC339524 | chr1: 87595448-87602350 | RNA gene | 0.06287 | 0.1582 |
| C3orf66 | chr3: 108897012-108904107 | RNA gene | 0.05678 | 0.1581 |
| HCG27 | chr6: 31165537-31171744 | RNA gene | 0.05556 | 0.1547 |
| RRN3P2 | chr16: 29086163-29128036 | pseudogene | 0.03989 | 0.1481 |
| C6orf122 | chr6: 170188886-170198921 | RNA gene | 0.0395 | 0.1466 |
| CFLP1 | chr10: 89578070-89605365 | pseudogene | 0.04513 | 0.1466 |
| LOC647946 | chr18: 36786888-37331959 | RNA gene | 0.04448 | 0.1445 |
| LOC100271722 | chr22: 46435789-46440748 | RNA gene | 0.05939 | 0.1378 |
| HOTAIR | chr12: 54356098-54362515 | RNA gene | 0.04097 | 0.1141 |
| LOC619207 | chr10: 135267432-135281949 | pseudogene | 0.03898 | 0.09046 |
| ASFMR1 | chrX: 146990949-147003676 | RNA gene | 0.03246 | 0.08789 |
| ALOXI2P2 | chr17: 6756895-6803667 | pseudogene | 0.03451 | 0.0801 |
| LOC100303728 | chrX: 118599997-118603083 | RNA gene | 0.03094 | 0.07179 |

Displayed genes showed an RMS-normalized RPKM ratio lower than 2 and were marked based on expression level in 3138 cells.

To validate the RNA Sequencing data, primers were designed for 7 of the differentially modulated lncRNAs (listed in Table 2 above) and greater than 2-fold up-regulation for each of them in the metastatic vs. non-metastatic xenografts was confirmed (FIG. 1B).

Among the differentially expressed lncRNAs, the transcript with highest expression in the metastatic xenograft was LOC728606, a previously uncharacterized gene. This transcript showed a similar magnitude of fold-change with 2 previously known oncogenic lncRNAs (FIG. 1B). LOC728606, flanked by AQP4 (Aquaporin-4) and KCTD1 (Potassium channel tetramerisation domain containing-1) loci, is transcribed to generate a 2598 bp RNA containing 2 exons (FIG. 1C), and is classified as a “long intergenic non-coding RNA” based on Ensembl algorithm (www.ensembl.org). ORF Finder (www.ncbi.nlm.nih.gov/gorf/gorf.html) revealed that the transcript is composed of non-translatable regions for at least 84% of its length (FIG. 1D). Test-code software (17) confirmed that the RNA does not encode a protein (p<0.01), and PepBank (Shtatland T, et al. PepBank—a database of peptides based on sequence text mining and public peptide data sources. BMC bioinformatics. 2007; 8:280) failed to identify any human peptide matching any ORF of this locus. Transcript length and sequence was confirmed by comparative analysis of multiple clones (as shown in Table 6).

TABLE 6
Summary of All Sequenced Clones from LOC728606 (PCAT18).

| | | | | | | Base |
|---|---|---|---|---|---|---|
| | | | From | From | | differences |
| | | | bp to | bp to | Accession | relative to |
| cDNA | | Match | bp in | bp in | match over | genome |
| accession | Tissue | mRNA | mRNA | accs. | (% length) | (% identity) |
| AK056805 | Prostate | AS | 1 to | 2 to | 2598/2598 | 9 diff |
| | | | 2597 | 2598 | (100%) | (99.7% id) |
| DA865211 | Prostate | AS | 1 to | 2 to | 825/825 | 12 diff |
| | | | 824 | 826 | (100%) | (98.6% id) |
| BX119491 | poorly differentiated | AS | 245 to | 458 to | 444/444 | 0 diff |
| | adenocarcinoma, stomach | | 688 | 15 | (100%) | (100% id) |
| AI685598 | Prostate | AS | 245 to | 452 to | 443/468 | 0 diff |
| | | | 687 | 10 | (94%) | (100% id) |
| FN152668 | breast carcinoma | AS | 358 to | 1 to | 90/90 | 0 diff |
| | | | 447 | 90 | (100%) | (100% id) |
| AI926047 | poorly differentiated | AS | 409 to | 290 to | 283/290 | 0 diff |
| | adenocarcinoma with | | 690 | 8 | (97%) | (100% id) |
| | signet ring cell | | | | | |
| | features, stomach | | | | | |
| AA635604 | Prostate | AS | 428 to | 264 to | 264/264 | 0 diff |
| | | | 691 | 1 | (100%) | (100% id) |
| DB329187 | Prostate | AS | 2162 to | 439 to | 439/444 | 0 diff |
| | | | 2597 | 1 | (98%) | (100% id) |

All known clones matching the LOC728606 sequence (NR_024259.1) were searched through the AceView database. Eight sequences were found, five of which were from prostate tissue and three from neoplastic tissues. The 5′ end of the gene is confirmed by independent readings. The AK056805 clone was generated using a 5′ oligo-capping method and polydT primers (Watahiki A. et al., PloS one. 2011; 6(9):e24950). The reference sequence was derived from AK056805.1 and DA865211. Match mRNA is antisense strand (AS) for all reads. We analyzed the polyA signal of the NP_024259.1 and AK056805.1 clones. A PolyA signal, AATAAA, was identified −25 to −18 bases from the 3′ end.

### Example 2: Expression Analysis of JUPITER (Also Known as PCAT18)

The expression of LOC728606 was investigated in publically available databases, for example, the Oncomine™ (FIG. 2A) and cBio (FIG. 28) databases. LOC728606 expression profiles were mined on Oncomine and Gene Expression Omnibus (GEO) databases, which include large collections of microarray data from human samples. LOC728606 is significantly up-regulated in PCa vs, normal tissue in both the Oncomine™ (FIG. 2A) and cBio (FIG. 28) databases. The data from the Oncomine™ analysis is summarized below in Table 7.

TABLE 7
Summary of all Oncomine ™ Outputs for LOC728606 in PCa, with p
value >0.01 and/or fold change <2.

| Study | Comparison | P value | Fold Change | Samples |
|---|---|---|---|---|
| Arredouani | PCa vs. normal | 0.043 | 2.0 | 21 |
| | prostate | | | |
| Arredouani | ERG rearrangement | 0.075 | 2.0 | 13 |
| | vs. no rearrangment | | | |
| Bittner | PCa-smoker vs. non- | 0.503 | −1.0 | 46 |
| | smoker | | | |
| Bittner | Acinar PCa-Grade 2 | 0.750 | −1.80 | 10 |
| | vs. grade 3 | | | |
| Bittner | PCa-Grade 2/3 vs. | 0.761 | N.A. | 46 |
| | grade 3 | | | |
| Bittner | PCa-Stage 2/3 vs. | 0.750 | N.A. | 43 |
| | stage 4 | | | |
| Bittner | Acinar PCa-smoker | 0.903 | −2.2 | 11 |
| | vs. non-smoker | | | |

All PCa studies with patient data included in the Oncomine™ database were selected.

In addition, this LOC728606 gene is significantly over-expressed in normal prostate compared to 11 other benign tissues (FIG. 2C) and in PCa compared to 15 other neoplastic tissues (FIG. 2D). Based on its chromosomal location and prostate cancer-specificity, this new gene was originally called JUPITER (Just Uncoding, Prostate-specific, Intergenic Transcript located on Eighteen chromosome Region q11.2). The HUGO Gene Nomenclature Committee has officially named this noncoding RNA PCAT18.

For further validation, the expression levels of JUPITER were analyzed in human prostate tissue and plasma samples using quantitative PCR (QPCR). JUPITER was highly up-regulated (8.8-11.1 fold, p<0.001) in both low-Gleason and high-Gleason PCa samples, compared to benign prostatic hyperplasia (BPH) (FIG. 3A). Therefore, JUPITER up-regulation is not a mere function of prostate cells' hyper-proliferation.

Based on its cancer and tissue-specificity, it was determined whether LOC728606 could be detected in plasma samples, and if it could be exploited as a biomarker for disease detection and monitoring. Plasma samples from normal individuals and those with localized PCa or mCRPC (n-25 per group, patients' characteristics summarized in Table 1) were analyzed. The results indicate a positive correlation between plasma LOC728606 levels and disease stage and show a stepwise increase in JUPITER expression (FIG. 3(B), p<0.01 for linear trend test), with significantly higher levels in mCRPC compared to all other categories. JUPITER expression (measured by QPCR) was also significantly elevated in 5 well-known human prostate cancer cell lines compared to a BPH cell line (BPH-1); see FIG. 3C.

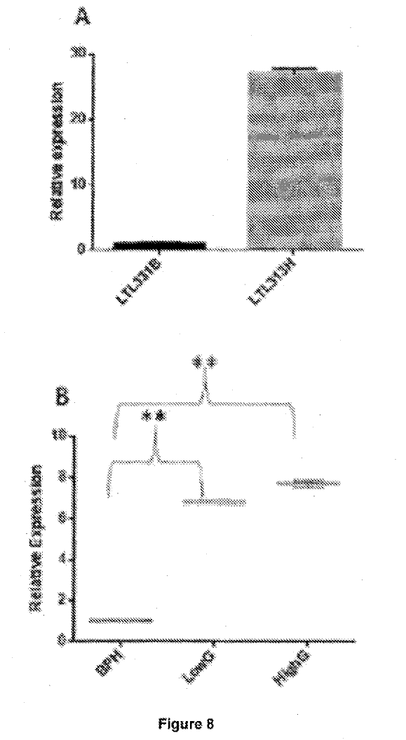

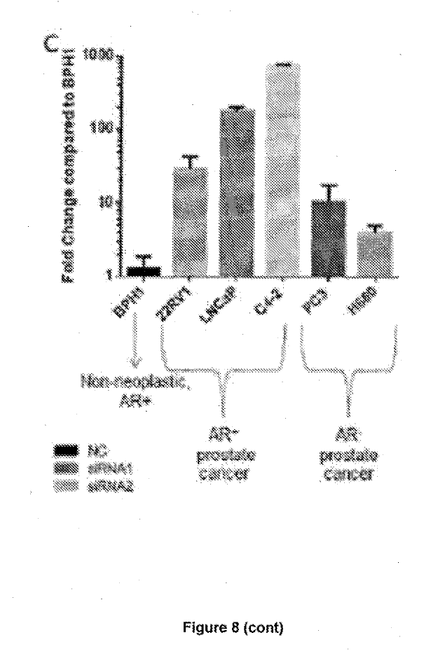

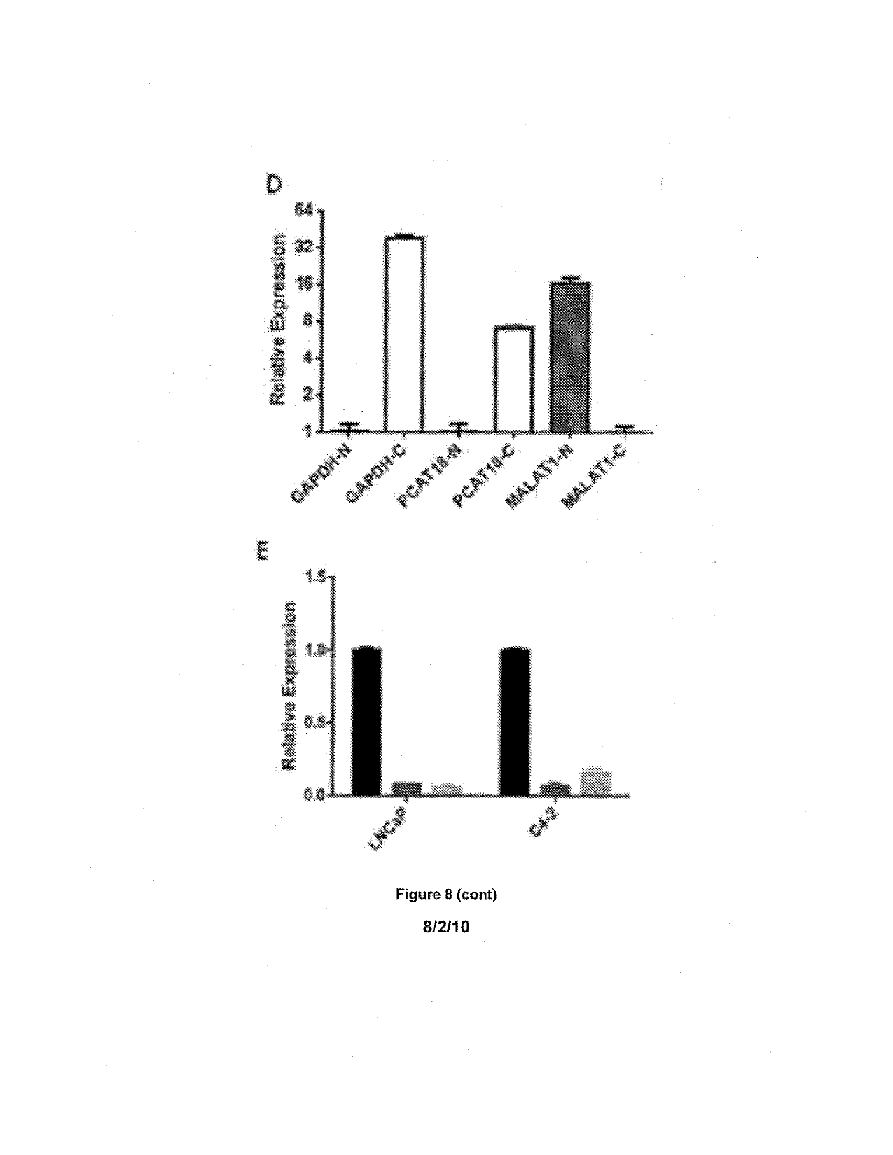

To further strengthen the above analysis, a new set of primers and a different qPCR methodology was used to confirm the LOC728606 expression profile in both preclinical and clinical samples (FIGS. 8(A) and 8(B)). In light of these data, this gene was officially named PCAT18 (Prostate Cancer-Associated Transcript-18) by the HUGO Gene Nomenclature Committee.

### Example 3: Identification of Transcripts Associated with JUPITER

Based on its expression profile, it was hypothesized that JUPITER might contribute to PCa clinical characteristics and interact with known oncogenic pathways. To this end, significance analysis of microarray data (SAM) was used to identify PCAT18-associated to transcripts. A dataset collecting RNA sequencing data and clinical Information on 131 PCa samples and 29 normal prostate tissues was exploited for this purposes. Analysis of this large dataset further confirmed that PCAT18 is significantly (p<0.001) up-regulated in PCa vs normal prostate (data not shown). SAM revealed 402 genes positively and significantly associated with PCAT18/JUPITER expression in PCa samples, as shown in Table 8 below. One or more than one of the genes listed in Table 8 may be used in combination with PCAT18 in order to diagnose or treat prostate cancer using the methods as described herein.

TABLE 8
PCAT18-associated expression signature.

| | |
|---|---|
| ACACA | acetyl-CoA carboxylase alpha |
| ACADL | acyl-CoA dehydrogenase, long chain |
| ACN9 | ACN9 homolog (S. cerevisiae) |
| ACP6 | acid phosphatase 6, lysophosphatidic |
| ACSM1 | acyl-CoA synthetase medium-chain family member 1 |
| ACSM3 | acyl-CoA synthetase medium-chain family member 3 |
| ACSS1 | acyl-CoA synthetase short-chain family member 1 |
| ACY1 | aminoacylase 1 |
| ADARB2 | adenosine deaminase, RNA-specific, B2 (RED2 homolog rat) |
| ADRB1 | adrenergic, beta-1-, receptor |
| ADRB2 | adrenergic, beta-2-, receptor, surface |
| AGA | aspartylglucosaminidase |
| AGAP1 | ArfGAP with GTPase domain, ankyrin repeat and PH domain 1 |
| AGFG2 | ArfGAP with FG repeats 2 |
| AK2P2 | adenylate kinase 2 pseudogene 2 |
| ALDH1A3 | aldehyde dehydrogenase 1 family, member A3 |
| ALG14 | asparagine-linked glycosylation 14 homolog (S. cerevisiae) |
| ALKBH2 | alkB, alkylation repair homolog 2 (E. coli) |
| ALMS1P | Alstrom syndrome 1 pseudogene |
| ANAPC5 | anaphase promoting complex subunit 5 |
| ANK3 | ankyrin 3, node of Ranvier (ankyrin G) |
| ANKRD37 | ankyrin repeat domain 37 |
| ANKRD5 | ankyrin repeat domain 5 |
| AP2S1 | adaptor-related protein complex 2, sigma 1 subunit |
| APOF | apolipoprotein F |
| ARF4P3 | ADP-ribosylation factor 4 pseudogene 3 |
| ARHGAP28 | Rho GTPase activating protein 28 |
| ATPSG2 | ATP synthase, H+ transporting, mitochondrial F0 complex, subunit C2 (subunit 9) |
| ATP8A1 | ATPase, aminophospholipid transporter (APLT), class I, type 8A, member 1 |
| ATPIF1 | ATPase inhibitory factor 1 |
| BAIAP3 | BAI1-associated protein 3 |
| BCAM | basal cell adhesion molecule (Lutheran blood group) |
| BEND4 | BEN domain containing 4 |
| BIK | BCL2-interacting killer (apoptosis-inducing) |
| BOLA3 | bolA homolog 3 (E. coli) |
| BPHL | biphenyl hydrolase-like (serine hydrolase) |
| BTBD11 | BTB (POZ) domain containing 11 |
| C10orf75 | chromosome 10 open reading frame 75 |
| C11orf10 | chromosome 11 open reading frame 10 |
| C11orf75 | chromosome 11 open reading frame 75 |
| C12orf60 | chromosome 12 open reading frame 60 |
| C14orf149 | chromosome 14 open reading frame 149 |
| C15orf23 | chromosome 15 open reading frame 23 |
| C15orf33 | chromosome 15 open reading frame 33 |
| C15orf61 | chromosome 15 open reading frame 61 |
| C16orf13 | chromosome 16 open reading frame 13 |
| C16orf70 | chromosome 16 open reading frame 70 |
| C17orf61 | chromosome 17 open reading frame 61 |
| C17orf79 | chromosome 17 open reading frame 79 |
| C18orf22 | chromosome 18 open reading frame 22 |
| C19orf46 | chromosome 19 open reading frame 46 |
| C19orf48 | chromosome 19 open reading frame 48 |
| C1orf66 | chromosome 1 open reading frame 66 |
| C2 | complement component 2 |
| C20orf196 | chromosome 20 open reading frame 196 |
| C20orf3 | chromosome 20 open reading frame 3 |
| C20orf96 | chromosome 20 open reading frame 96 |
| C22orf32 | chromosome 22 open reading frame 32 |
| C2orf72 | chromosome 2 open reading frame 72 |
| C2orf76 | chromosome 2 open reading frame 76 |
| C2orf79 | chromosome 2 open reading frame 79 |
| C3orf25 | chromosome 3 open reading frame 25 |
| C4orf14 | chromosome 4 open reading frame 14 |
| C4orf47 | chromosome 4 open reading frame 47 |
| C5orf49 | chromosome 5 open reading frame 49 |
| C6orf108 | chromosome 6 open reading frame 108 |
| C6orf124 | chromosome 6 open reading frame 124 |
| C6orf57 | chromosome 6 open reading frame 57 |
| C7orf53 | chromosome 7 open reading frame 53 |
| C8orf34 | chromosome 8 open reading frame 34 |
| C8orf45 | chromosome 8 open reading frame 45 |
| C9orf152 | chromosome 9 open reading frame 152 |
| C9orf43 | chromosome 9 open reading frame 43 |
| CAMK1 | calcium/calmodulin-dependent protein kinase I |
| CAMK2B | calcium/calmodulin-dependent protein kinase II beta |
| CAMKK2 | calcium/calmodulin-dependent protein kinase kinase 2, beta |
| CAPN9 | calpain 9 |
| CATSPER2 | cation channel, sperm associated 2 |
| CATSPER2P1 | cation channel, sperm associated 2 pseudogene 1 |
| CBS | cystathionine-beta-synthase |
| CCDC110 | coiled-coil domain containing 110 |
| CCDC149 | coiled-coil domain containing 149 |
| CCDC51 | coiled-coil domain containing 51 |
| CCT3 | chaperonin containing TCP1, subunit 3 (gamma) |
| CDK3 | cyclin-dependent kinase 3 |
| CDK5 | cyclin-dependent kinase 5 |
| CECR5 | cat eye syndrome chromosome region, candidate 5 |
| CECR7 | cat eye syndrome chromosome region, candidate 7 (non-protein coding) |
| CGREF1 | cell growth regulator with EF-hand domain 1 |
| CHDH | choline dehydrogenase |
| CHKA | choline kinase alpha |
| CHMP4C | chromatin modifying protein 4C |
| CHRNA2 | cholinergic receptor, nicotinic, alpha 2 (neuronal) |
| CISD3 | CDGSH iron sulfur domain 3 |
| CLDN8 | claudin 8 |
| CLEC18A | C-type lectin domain family 18, member A |
| CLEC18B | C-type lectin domain family 18, member B |
| CLEC18C | C-type lectin domain family 18, member C |
| CMTM4 | CKLF-like MARVEL transmembrane domain containing 4 |
| CNTN3 | contactin 3 (plasmacytoma associated) |
| CORO1B | coronin, actin binding protein, 1B |
| CPNE7 | copine VII |
| CREB3L4 | cAMP responsive element binding protein 3-like 4 |
| CRYL1 | crystallin, lambda 1 |
| CYB5A | cytochrome b5 type A (microsomal) |
| DAK | dihydroxyacetone kinase 2 homolog (S. cerevisiae) |
| DBI | diazepam binding inhibitor (GABA receptor modulator, acyl-CoA binding protein) |
| DCXR | dicarbonyl/L-xylulose reductase |
| DECR2 | 2,4-dienoyl CoA reductase 2, peroxisomal |
| DKFZP686I15217 | hypothetical LOC401232 |
| DNAH5 | dynein, axonemal, heavy chain 5 |
| DNAH7 | dynein, axonemal, heavy chain 7 |
| DOPEY2 | dopey family member 2 |
| DPY19L2P4 | dpy-19-like 2 pseudogene 4 (C. elegans) |
| DSC2 | desmocollin 2 |
| DUS1L | dihydrouridine synthase 1-like (S. cerevisiae) |
| EDEM3 | ER degradation enhancer, mannosidase alpha-like 3 |
| EGF | epidermal growth factor |
| EIF4EBP1 | eukaryotic translation initiation factor 4E binding protein 1 |
| ELL3 | elongation factor RNA polymerase II-like 3 |
| ELMO3 | engulfment and cell motility 3 |
| ELOVL5 | ELOVL family member 5, elongation of long chain fatty acids (FEN1/Elo2, |
| | SUR4/Elo3-like, yeast) |
| ENOX1 | ecto-NOX disulfide-thiol exchanger 1 |
| EPB41L4B | erythrocyte membrane protein band 4.1 like 4B |
| ERBB3 | v-erb-b2 erythroblastic leukemia viral oncogene homolog 3 (avian) |
| ERGIC1 | endoplasmic reticulum-golgi intermediate compartment (ERGIC) 1 |
| ESRP2 | epithelial splicing regulatory protein 2 |
| EXOSC5 | exosome component 5 |
| FAH | fumarylacetoacetate hydrolase (fumarylacetoacetase) |
| FAM128A | family with sequence similarity 128, member A |
| FAM13C | family with sequence similarity 13, member C |
| FAM19A4 | family with sequence similarity 19 (chemokine (C-C motif)-like), member A4 |
| FAM81A | family with sequence similarity 81, member A |
| FASN | fatty acid synthase |
| FBXL8 | F-box and leucine-rich repeat protein 8 |
| FHIT | fragile histidine triad gene |
| FLJ27352 | hypothetical LOC145788 |
| FLJ46552 | FLJ46552 protein |
| FRMPD3 | FERM and PDZ domain containing 3 |
| FZD8 | frizzled homolog 8 (Drosophila) |
| GALNT3 | UDP-N-acetyl-alpha-D-galactosamine:polypeptide N- |
| | acetylgalactosaminyltransferase 3 (GalNAc-T3) |
| GGCT | gamma-glutamylcyclotransferase |
| GJB1 | gap junction protein, beta 1, 32 kDa |
| GLYATL1 | glycine-N-acyltransferase-like 1 |
| GMPPB | GDP-mannose pyrophosphorylase B |
| GRB14 | growth factor receptor-bound protein 14 |
| GRPR | gastrin-releasing peptide receptor |
| GTF3C1 | general transcription factor IIIC, polypeptide 1, alpha 220 kDa |
| H2AFJ | H2A histone family, member J |
| HEBP2 | heme binding protein 2 |
| HIST3H2A | histone cluster 3, H2a |
| HKR1 | HKR1, GLI-Kruppel zinc finger family member |
| HMG20B | high-mobility group 20B |
| HOXA9 | homeobox A9 |
| HPN | hepsin |
| HSF4 | heat shock transcription factor 4 |
| ICT1 | immature colon carcinoma transcript 1 |
| IGSF5 | immunoglobulin superfamily, member 5 |
| IGSF8 | immunoglobulin superfamily, member 8 |
| IL20RA | interleukin 20 receptor, alpha |
| ILDR1 | immunoglobulin-like domain containing receptor 1 |
| IMPDH2 | IMP (inosine 5′-monophosphate) dehydrogenase 2 |
| IQCH | IQ motif containing H |
| IVD | isovaleryl-CoA dehydrogenase |
| KATNB1 | katanin p80 (WD repeat containing) subunit B 1 |
| KCNH6 | potassium voltage-gated channel, subfamily H (eag-related), member 6 |
| KCTD1 | potassium channel tetramerisation domain containing 1 |
| KIAA0182 | KIAA0182 |
| KIAA1543 | KIAA1543 |
| KIAA1549 | KIAA1549 |
| KIAA1804 | mixed lineage kinase 4 |
| KLK15 | kallikrein-related peptidase 15 |
| KRT18 | keratin 18 |
| KRT18P13 | keratin 18 pseudogene 13 |
| KRT18P17 | keratin 18 pseudogene 17 |
| KRT18P19 | keratin 18 pseudogene 19 |
| KRT18P24 | keratin 18 pseudogene 24 |
| KRT18P26 | keratin 18 pseudogene 26 |
| KRT18P28 | keratin 18 pseudogene 28 |
| KRT18P30 | keratin 18 pseudogene 30 |
| KRT18P33 | keratin 18 pseudogene 33 |
| KRT18P34 | keratin 18 pseudogene 34 |
| KRT18P40 | keratin 18 pseudogene 40 |
| KRT18P42 | keratin 18 pseudogene 42 |
| KRT18P46 | keratin 18 pseudogene 46 |
| LASS4 | LAG1 homolog, ceramide synthase 4 |
| LEKR1 | leucine, glutamate and lysine rich 1 |
| LFNG | LFNG O-fucosylpeptide 3-beta-N-acetylglucosaminyltransferase |
| LOC100127980 | hypothetical protein LOC100127980 |
| LOC100128332 | hypothetical protein LOC100128332 |
| LOC100128737 | hypothetical LOC100128737 |
| LOC100128841 | similar to hCG39453 |
| LOC100129387 | hypothetical LOC100129387 |
| LOC100129514 | hypothetical LOC100129514 |
| LOC100131047 | hypothetical protein LOC100131047 |
| LOC100131199 | hypothetical LOC100131199 |
| LOC100132111 | hypothetical LOC100132111 |
| LOC100133580 | hypothetical protein LOC100133580 |
| LOC100134348 | similar to TBC1 domain family member 3 (Rab GTPase-activating protein PRC17) |
| | (Prostate cancer gene 17 protein) (TRE17 alpha protein) |
| LOC389768 | potassium channel tetramerisation domain containing 1 pseudogene |
| LOC391811 | similar to polymerase (DNA directed), delta 2, regulatory subunit |
| LOC399815 | chromosome 10 open reading frame 88 pseudogene |
| LOC440335 | hypothetical LOC440335 |
| LOC442249 | similar to keratin 18 |
| LOC642384 | hypothetical LOC642384 |
| LOC642590 | similar to spermine synthase |
| LOC643327 | hypothetical LOC643327 |
| LOC643637 | similar to hCG1729961 |
| LOC646347 | similar to spermine synthase |
| LOC653380 | TBC1 domain family member 3C-like protein ENSP00000341742 |
| LOC728431 | hypothetical LOC728431 |
| LOC729774 | hypothetical LOC729774 |
| LOC729779 | similar to phosphoserine aminotransferase |
| LOC81691 | exonuclease NEF-sp |
| LRGUK | leucine-rich repeats and guanylate kinase domain containing |
| LRIG1 | leucine-rich repeats and immunoglobulin-like domains 1 |
| LRRC26 | leucine rich repeat containing 26 |
| LRRC63 | leucine rich repeat containing 63 |
| LRRIQ1 | leucine-rich repeats and IQ motif containing 1 |
| LYPLA2P1 | lysophospholipase II pseudogene 1 |
| MAP1D | methionine aminopeptidase 1D |
| MBOAT2 | membrane bound O-acyltransferase domain containing 2 |
| MCCC2 | methylcrotonoyl-CoA carboxylase 2 (beta) |
| MDH2 | malate dehydrogenase 2, NAD (mitochondrial) |
| MED12L | mediator complex subunit 12-like |
| METTL9 | methyltransferase like 9 |
| MMP26 | matrix metallopeptidase 26 |
| MOSC1 | MOCO sulphurase C-terminal domain containing 1 |
| MPND | MPN domain containing |
| MRPL12 | mitochondrial ribosomal protein L12 |
| MRPL24 | mitochondrial ribosomal protein L24 |
| MRPS24 | mitochondrial ribosomal protein S24 |
| MRPS33 | mitochondrial ribosomal protein S33 |
| MYBPC1 | myosin binding protein C, slow type |
| MYRIP | myosin VIIA and Rab interacting protein |
| NAAA | N-acylethanolamine acid amidase |
| NDUFA8 | NADH dehydrogenase (ubiquinone) 1 alpha subcomplex, 8, 19 kDa |
| NDUFB10 | NADH dehydrogenase (ubiquinone) 1 beta subcomplex, 10, 22 kDa |
| NEIL1 | nei endonuclease VIII-like 1 (E. coli) |
| NME4 | non-metastatic cells 4, protein expressed in |
| NMRAL1 | NmrA-like family domain containing 1 |
| NPY | neuropeptide Y |
| NSUN7 | NOP2/Sun domain family, member 7 |
| NUDT9 | nudix (nucleoside diphosphate linked moiety X)-type motif 9 |
| NUPR1 | nuclear protein, transcriptional regulator, 1 |
| NWD1 | NACHT and WD repeat domain containing 1 |
| OAZ3 | ornithine decarboxylase antizyme 3 |
| OCEL1 | occludin/ELL domain containing 1 |
| OR51A7 | olfactory receptor, family 51, subfamily A, member 7 |
| OR51F1 | olfactory receptor, family 51, subfamily F, member 1 |
| OR51F2 | olfactory receptor, family 51, subfamily F, member 2 |
| OR51G2 | olfactory receptor, family 51, subfamily G, member 2 |
| OR51L1 | olfactory receptor, family 51, subfamily L, member 1 |
| OR51T1 | olfactory receptor, family 51, subfamily T, member 1 |
| OVGP1 | oviductal glycoprotein 1, 120 kDa |
| OXSM | 3-oxoacyl-ACP synthase, mitochondrial |
| PAOX | polyamine oxidase (exo-N4-amino) |
| PCA3 | prostate cancer antigen 3 (non-protein coding) |
| PCBD1 | pterin-4 alpha-carbinolamine dehydratase/dimerization cofactor of hepatocyte nuclear |
| | factor 1 alpha |
| PCGEM1 | prostate-specific transcript 1 (non-protein coding) |
| PCGF1 | polycomb group ring finger 1 |
| PCGF3 | polycomb group ring finger 3 |
| PCTP | phosphatidylcholine transfer protein |
| PDCD2L | programmed cell death 2-like |
| PDE3B | phosphodiesterase 3B, cGMP-inhibited |
| PDE9A | phosphodiesterase 9A |
| PECI | peroxisomal D3,D2-enoyl-CoA isomerase |
| PET112L | PET112-like (yeast) |
| PEX10 | peroxisomal biogenesis factor 10 |
| PEX7 | peroxisomal biogenesis factor 7 |
| PIGM | phosphatidylinositol glycan anchor biosynthesis, class M |
| PKN1 | protein kinase N1 |
| PLCB4 | phospholipase C, beta 4 |
| PMM1 | phosphomannomutase 1 |
| PODXL2 | podocalyxin-like 2 |
| POLD2 | polymerase (DNA directed), delta 2, regulatory subunit 50 kDa |
| POLN | polymerase (DNA directed) nu |
| POP7 | processing of precursor 7, ribonuclease P/MRP subunit (S. cerevisiae) |
| PPAPDC1B | phosphatidic acid phosphatase type 2 domain containing 1B |
| PPM1E | protein phosphatase, Mg2+/Mn2+ dependent, 1E |
| PPM1H | protein phosphatase, Mg2+/Mn2+ dependent, 1H |
| PPP1R9A | protein phosphatase 1, regulatory (inhibitor) subunit 9A |
| PPYR1 | pancreatic polypeptide receptor 1 |
| PRDM10 | PR domain containing 10 |
| PRDX4 | peroxiredoxin 4 |
| PRSS8 | protease, serine, 8 |
| PRTG | protogenin homolog (Gallus gallus) |
| PSTK | phosphoseryl-tRNA kinase |
| PTPN20A | protein tyrosine phosphatase, non-receptor type 20A |
| PTPN20B | protein tyrosine phosphatase, non-receptor type 20B |
| PTPRN2 | protein tyrosine phosphatase, receptor type, N polypeptide 2 |
| PYCR1 | pyrroline-5-carboxylate reductase 1 |
| RAB17 | RAB17, member RAS oncogene family |
| RAB3B | RAB3B, member RAS oncogene family |
| RAB3D | RAB3D, member RAS oncogene family |
| RAB3IP | RAB3A interacting protein (rabin3) |
| RABIF | RAB interacting factor |
| RAC3 | ras-related C3 botulinum toxin substrate 3 (rho family, small GTP binding protein |
| | Rac3) |
| REPS2 | RALBP1 associated Eps domain containing 2 |
| RG9MTD2 | RNA (guanine-9-) methyltransferase domain containing 2 |
| RIMKLA | ribosomal modification protein rimK-like family member A |
| RNLS | renalase, FAD-dependent amine oxidase |
| RORC | RAR-related orphan receptor C |
| RPL14P3 | ribosomal protein L14 pseudogene 3 |
| RPL22L1 | ribosomal protein L22-like 1 |
| RPL29P15 | ribosomal protein L29 pseudogene 15 |
| RPL29P30 | ribosomal protein L29 pseudogene 30 |
| RPL36 | ribosomal protein L36 |
| RPL7AP68 | ribosomal protein L7a pseudogene 68 |
| RPLP2P3 | ribosomal protein, large, P2 pseudogene 3 |
| RPS12P23 | ribosomal protein S12 pseudogene 23 |
| RPS19BP1 | ribosomal protein S19 binding protein 1 |
| RPS24 | ribosomal protein S24 |
| SATB2 | SATB homeobox 2 |
| SCAND3 | SCAN domain containing 3 |
| SCD | stearoyl-CoA desaturase (delta-9-desaturase) |
| SHANK2 | SH3 and multiple ankyrin repeat domains 2 |
| SLC19A1 | solute carrier family 19 (folate transporter), member 1 |
| SLC25A33 | solute carrier family 25, member 33 |
| SLC25A42 | solute carrier family 25, member 42 |
| SLC26A6 | solute carrier family 26, member 6 |
| SLC35F2 | solute carrier family 35, member F2 |
| SLC43A1 | solute carrier family 43, member 1 |
| SLC9A2 | solute carrier family 9 (sodium/hydrogen exchanger), member 2 |
| SMOX | spermine oxidase |
| SMPDL3B | sphingomyelin phosphodiesterase, acid-like 3B |
| SMS | spermine synthase |
| SNHG11 | small nucleolar RNA host gene 11 (non-protein coding) |
| SNORA18 | small nucleolar RNA, H/ACA box 18 |
| SNORA2A | small nucleolar RNA, H/ACA box 2A |
| SNORD104 | small nucleolar RNA, C/D box 104 |
| SNORD116-11 | small nucleolar RNA, C/D box 116-11 |
| SNORD35A | small nucleolar RNA, C/D box 35A |
| SNORD57 | small nucleolar RNA, C/D box 57 |
| SNORD74 | small nucleolar RNA, C/D box 74 |
| SNRPD2 | small nuclear ribonucleoprotein D2 polypeptide 16.5 kDa |
| SPAG6 | sperm associated antigen 6 |
| SPATA17 | spermatogenesis associated 17 |
| SPDEF | SAM pointed domain containing ets transcription factor |
| SPIN3 | spindlin family, member 3 |
| SPOCK1 | sparc/osteonectin, cwcv and kazal-like domains proteoglycan (testican) 1 |
| SREBF1 | sterol regulatory element binding transcription factor 1 |
| STX19 | syntaxin 19 |
| STYXL1 | serine/threonine/tyrosine interacting-like 1 |
| TAAR6 | trace amine associated receptor 6 |
| TARS2 | threonyl-tRNA synthetase 2, mitochondrial (putative) |
| TAS2R10 | taste receptor, type 2, member 10 |
| TBC1D3 | TBC1 domain family, member 3 |
| TBC1D3B | TBC1 domain family, member 3B |
| TBC1D3C | TBC1 domain family, member 3C |
| TBC1D3E | TBC1 domain family, member 3E |
| TBC1D3F | TBC1 domain family, member 3F |
| TBC1D3G | TBC1 domain family, member 3G |
| TBC1D3H | TBC1 domain family, member 3H |
| TBC1D4 | TBC1 domain family, member 4 |
| TDRKH | tudor and KH domain containing |
| TERC | telomerase RNA component |
| TGM3 | transglutaminase 3 (E polypeptide, protein-glutamine-gamma-glutamyltransferase) |
| TMED3 | transmembrane emp24 protein transport domain containing 3 |
| TMEFF2 | transmembrane protein with EGF-like and two follistatin-like domains 2 |
| TMEM144 | transmembrane protein 144 |
| TMEM223 | transmembrane protein 223 |
| TMEM27 | transmembrane protein 27 |
| TMEM5 | transmembrane protein 5 |
| TMPRSS11F | transmembrane protease, serine 11F |
| TMSB15A | thymosin beta 15a |
| TMTC4 | transmembrane and tetratricopeptide repeat containing 4 |
| TOM1L1 | target of myb1 (chicken)-like 1 |
| TP53TG1 | TP53 target 1 (non-protein coding) |
| TREX1 | three prime repair exonuclease 1 |
| TRIM3 | tripartite motif-containing 3 |
| TSPAN1 | tetraspanin 1 |
| TSSC1 | tumor suppressing subtransferable candidate 1 |
| TTC18 | tetratricopeptide repeat domain 18 |
| TTC6 | tetratricopeptide repeat domain 6 |
| TTLL12 | tubulin tyrosine ligase-like family, member 12 |
| TUBA3D | tubulin, alpha 3d |
| TUBA3E | tubulin, alpha 3e |
| TUT1 | terminal uridylyl transferase 1, U6 mRNA-specific |
| UAP1 | UDP-N-acteylglucosamine pyrophosphorylase 1 |
| UBE2E2 | ubiquitin-conjugating enzyme E2E 2 (UBC4/5 homolog, yeast) |
| UBXN8 | UBX domain protein 8 |
| USP54 | ubiquitin specific peptidase 54 |
| VLDLR | very low density lipoprotein receptor |
| WIBG | within bgcn homolog (Drosophila) |
| WWC1 | WW and C2 domain containing 1 |
| XKR6 | XK, Kell blood group complex subunit-related family, member 6 |
| XYLB | xylulokinase homolog (H. influenzae) |
| YIPF1 | Yip1 domain family, member 1 |
| ZADH2 | zinc binding alcohol dehydrogenase domain containing 2 |
| ZBTB37 | zinc finger and BTB domain containing 37 |
| ZBTB7B | zinc finger and BTB domain containing 7B |
| ZDHHC11 | zinc finger, DHHC-type containing 11 |
| ZDHHC23 | zinc finger, DHHC-type containing 23 |
| ZMYND12 | zinc finger, MYND-type containing 12 |
| ZNF30 | zinc finger protein 30 |
| ZNF485 | zinc finger protein 485 |
| ZNF511 | zinc finger protein 511 |
| ZNF643 | zinc finger protein 643 |
| ZNF692 | zinc finger protein 692 |
| ZNF697 | zinc finger protein 697 |
| ZNF862 | zinc finger protein 862 |

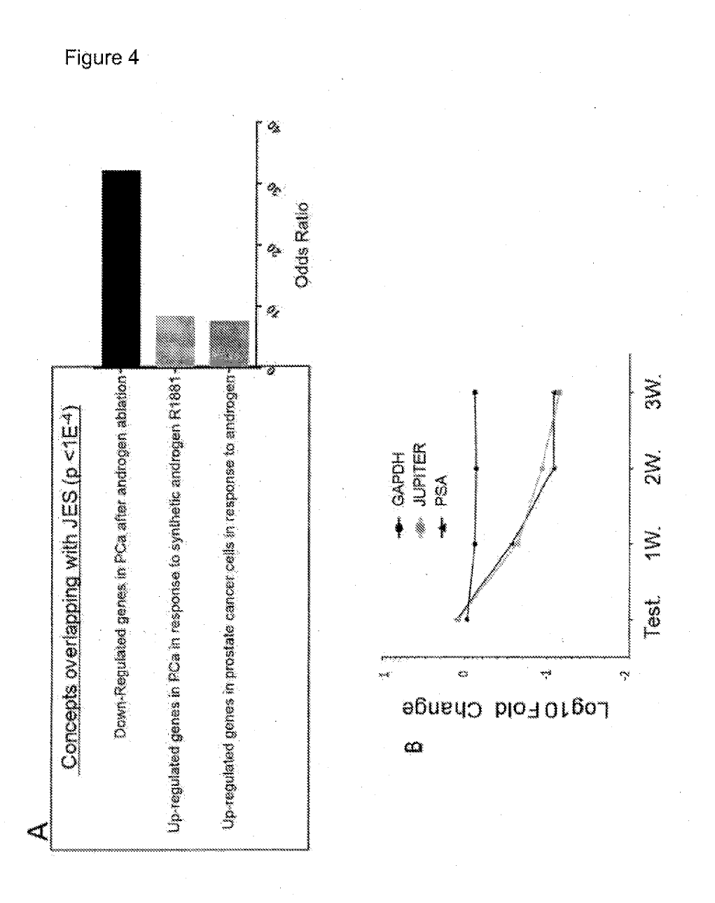

The above PCAT18-/JUPITER-associated expression signature (JES) was then uploaded into Oncomine to identify clinically significant associations and to perform pathway analysis. JES was consistently up-regulated in PCa vs. normal tissue and PCa vs. other neoplasms in several cancer studies, comprising more than 4000 human samples (see Table 9 below). Importantly, JES was significantly up-regulated in metastatic vs. primary PCa. Pathway analysis revealed that JES Is strongly associated with androgen receptor activation and differentiation of epithelial cells (FIG. 4A). To experimentally determine whether JUPITER expression is down-regulated following castration, we assessed its expression following to castration in a PCa xenograft model. As shown in FIG. 4B, there is a larger than 10-fold decrease in JUPITER expression by 3 weeks post-castration. This decrease is comparable to the one observed for PSA (16.6 vs. 14.7 fold change).

The second column (‘Studies’) in Table 9 above shows the number of independent studies showing up- or down-regulation of JES for a specific concept. Oncomine™ (Compendia Bioscience, Ann Arbor, Mich.) was used for the analysis.

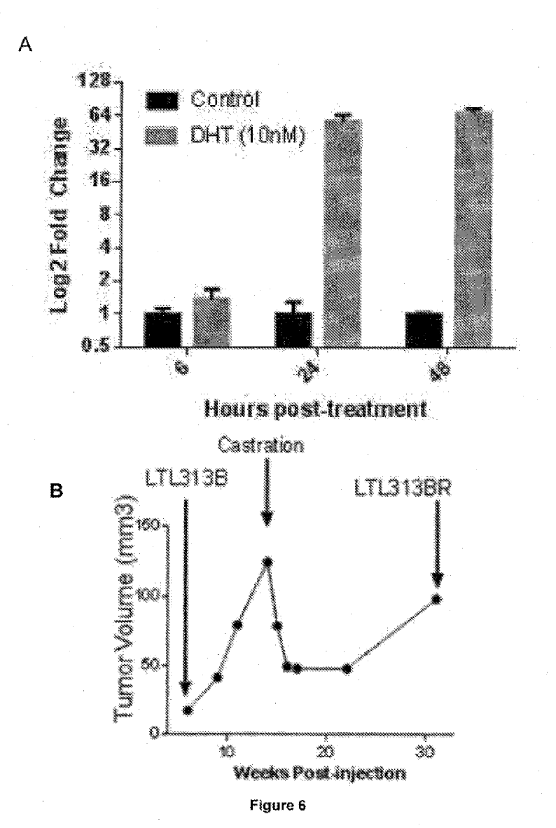

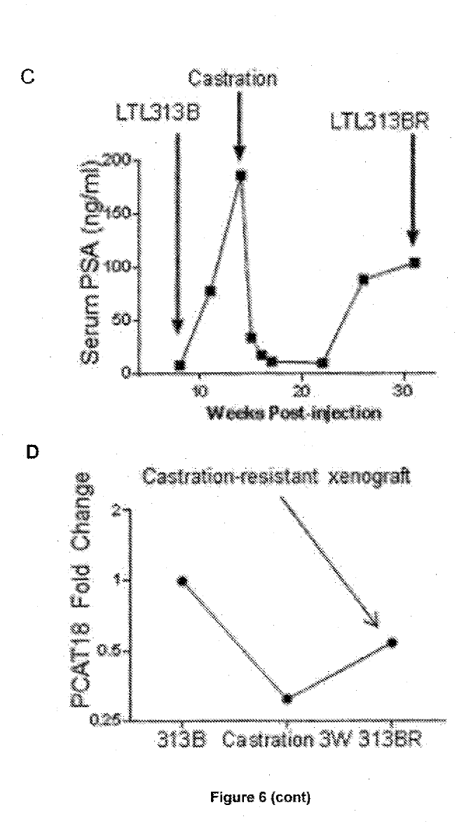

To gain insights into PCAT18 function, in vitro studies on the metastatic PCa-derived LNCaP cell line (Horoszewicz J S, et al. LNCaP model of human prostatic carcinoma. Cancer research. 1983; 43(4):1809-1818) was performed. In this model, dihydrotestosterone (DHT) treatment (24-48 h) induced a more than 50-fold up-regulation of PCAT18 expression (FIG. 6(A)). In keeping with this relatively late up-regulation, no androgen-receptor (AR) binding site in the PCAT18 putative promoter (Table 10 below, and data from ChIP-on-chip (Decker K F, et al. Persistent androgen receptor-mediated transcription in castration-resistant prostate cancer under androgen-deprived conditions. Nucleic acids research 2012; 40(21):10765-10779)) was found. This evidence suggests that androgen indirectly activates PCAT18 expression. To experimentally determine whether PCAT18 is downregulated following androgen ablation, PCAT18 expression was assessed following castration in two PCa xenograft models. The LTL331 model generates a typical androgen-dependent PCa (Lin D, et al., High fidelity patient-derived xenografts for accelerating prostate cancer discovery and drug development. Cancer research. 2013). In this model, androgen deprivation induced a dramatic PCAT18 down-regulation (FIG. 4(B)). The expression profile of PCAT18 was then investigated in a recently developed CRPC subline (LTL313BR). When the LTL313B xenograft is exposed to castrate levels of androgens for several weeks, it reproducibly generates an AR+ CRPC sub-line (FIGS. 6(B) and 6(C)) (Lin D, et al., Cancer research, 2013). In this model, castration induced PCAT18 down-regulation, but the emergence of the CRPC subline was associated with PCAT18 up-regulation (FIG. 6(D)).

TABLE 10
PCAT18 Promoter analysis

| Transcription Factor | Matrix |
|---|---|
| AP-2alphaA | [T00035] |
| C/EBPalpha | [T00105] |
| C/EBPbeta | [T00581] |
| c-Ets-1 | [T00112] |
| c-Ets-2 | [T00113] |
| c-Fos | [T00123] |
| c-Jun | [T00133] |
| c-Myb | [T00137] |
| CTF | [T00174] |
| Elk-1 | [T00250] |
| ER-alpha | [T00261] |
| FOXP3 | [T04280] |
| GATA-1 | [T00306] |
| GATA-2 | [T00308] |
| GR | [T05076] |
| GR-alpha | [T00337] |
| GR-beta | [T01920] |
| HNF-1A | [T00368] |
| Ik-1 | [T02702] |
| IRF-1 | [T00423] |
| NF-AT1 | [T01948] |
| NF-AT2 | [T01945] |
| NF-Y | [T00150] |
| Pax-5 | [T00070] |
| RelA | [T00594] |
| RXR-alpha | [T01345] |
| Sp1 | [T00759] |
| STAT4 | [T01577] |
| STAT5A | [T04683] |
| T3R-beta1 | [T00851] |
| TBP | [T00794] |
| TFII-I | [T00824] |
| USF2 | [T00878] |
| VDR | [T00885] |
| XBP-1 | [T00902] |
| YY1 | [T00915] |

Promoter analysis—from Chromosome 18 primary assembly (NC_000018.9); 1 Kb of 5′ flanking sequence was downloaded, immediately adjacent to the PCAT18 transcription start site. The sequence was uploaded in PROMO software (http://alggen.lsi.upc.es/cgi-bin/promo_v3/promo/promoinit.cgi?dirDB=TF_8.3), to identify human transcription factor binding sites (maximum dissimilarity: 5%). 36 transcription factors were identified, some with a known oncogenic role (c-Fos, c-Jun, STAT). The unique matrix that identifies each transcription factor is shown beside the official name. Notably, no AR (androgen-receptor) binding site was detected. These findings were confirmed by analyzing a ChIP-on-chip dataset comprising AR binding sites in LNCaP cells exposed to castrate levels of androgens or 10 nM DHT23. The authors identified a set of androgen-dependent and independent AR binding sites throughout the genome. Exploring these datasets, the closest AR binding site was 29.9 Kb from the PCAT18 transcription starting site.

### Example 4: Functional Characterization of PCAT18

The functional relevance of PCAT18 in PCa cells was then determined. To this aim, PCAT18's expression levels in a panel of prostate cell lines was measured. In keeping with the previous data. PCAT18 expression was higher in AR-positive than in AR-negative PCa cells (FIG. 8(C)) Among AR-positive cells, PCAT18 levels incrementally increased from non-neoplastic (BPH1), to androgensensitive (22Rv1, LNCaP) and androgen-insensitive (C4-2) PCa cells. LNCaP and its castrate-resistant sub-line C4-2 (Wu H C, et al. Derivation of androgen-independent human LNCaP prostatic cancer cell sublines: role of bone stromal cells. International journal of cancer Journal international du cancer. 1994; 57(3):406-412) were, therefore, selected for lncRNA characterization and functional studies. RNA fractionation and quantification experiments revealed that PCAT18 is mainly located in the cytoplasm of PCa cells (FIG. 8(D)). Indeed, the PCAT18 expression profile more similar to the protein-coding RNA GAPDH than to the nuclear-retained lncRNA MALAT1 (Miyagawa R, et al. Identification of cis- and trans-acting factors involved in the localization of MALAT-1 noncoding RNA to nuclear speckles. Rna. 2012; 18(4):738-751).

### Example 5: Effect of Silencing PCAT18 Using siRNAs

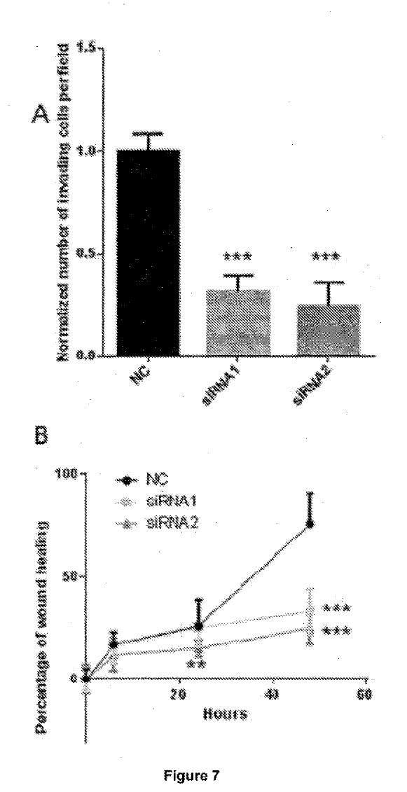

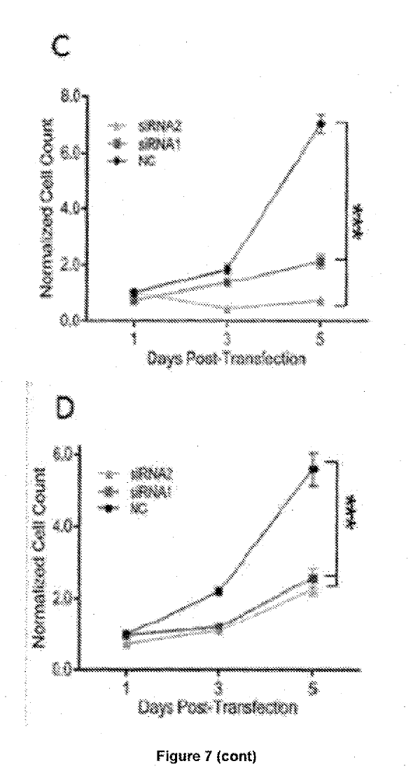

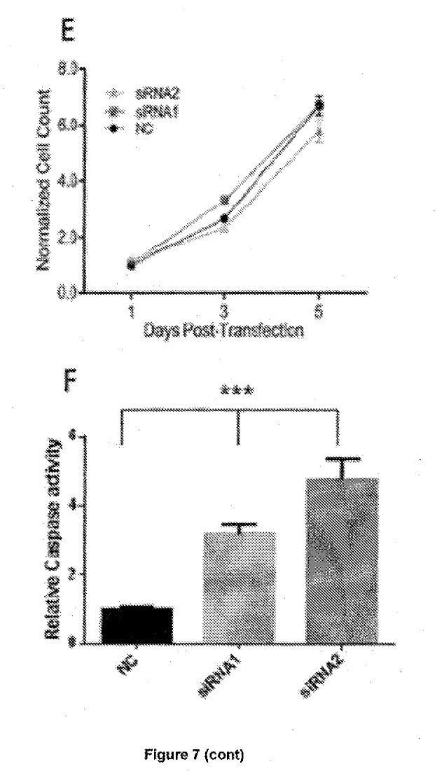

Two small-interfering RNAs (siRNAs) (siRNA1 and siRNA2, the sequences of which are set out in Table 11) were used in a human prostate cancer cell line (C4-2) to silence JUPTER/PCAT18 expression. These two siRNAs induced greater than 80% gene knockdown at a 2 nM concentration (FIG. 8(E)) (Kim D H, et al. Synthetic dsRNA Dicer substrates enhance RNAi potency and efficacy. Nature biotechnology. 2005; 23(2):222-226). PCAT18 silencing (24-48 h) significantly inhibited PCa cell invasion and migration (FIGS. 7(A) and 7(B); see also FIG. 3(D)). At later time points (5 days), PCAT18 silencing induced a significant growth inhibition in both LNCaP and C4-2 cells (FIGS. 7(C) and 7(D)) (for C4-2 cells, see also FIG. 3(E)), with no effect on non-neoplastic BPH1 cells (FIG. 7(E)). Prolonged PCAT18 silencing (5 days) also triggered caspase 3/7 activation (FIG. 7(F)).

TABLE 11
Nucleotide Sequences of siRNA1 and siRNA2.

| | SEQ | |
|---|---|---|
| | ID | |
| siRNA | NO. | Sequence |
| siRNA1 | 22 | AGCAGGAACAUUCCAAUAGAAGAAAUAUUGGAAUGU |
| siRNA2 | 23 | GCAACAUGACCUACAGUUAAUGAGUAACUGUAGGUC |

### Discussion of Examples

The above Examples revealed 153 up-regulated and 77 down-regulated lncRNAs in the metastatic versus non-metastatic xenografts. The most up-regulated transcript was an uncharacterized lncRNA, PCAT18 (also referred to as JUPITER), is characterized herein. Database analysis revealed that PCAT18 is specifically expressed in normal prostate compared to 11 normal tissues (p<0.05) and specifically up-regulated in PCa compared to 15 other neoplasms (p<0.001). Cancer-specific up-regulation of PCAT18 was confirmed on an independent dataset of PCa and benign prostatic hyperplasia samples (p<0.001). In addition, PCAT18 was detectable in plasma samples and increased incrementally from normal individuals to those with localized and metastatic PCa (p<0.01). Co-expression analysis allowed us to identify a PCAT18-associated expression signature (PES or JES for J PCAT18-associated expression), which is highly PCa-specific and is activated in metastatic vs. primary PCa samples (p<1E-4, odds ratio>2). Pathway analysis revealed that PES is significantly associated with androgen receptor activation. PCAT18 expression was experimentally confirmed to be dramatically decreased upon PCa xenograft castration.

Due to the slow-growing nature of this disease, PCa samples are often composed of multi-clonal subpopulations, each with a different mutational spectrum and metastatic potential (18). Molecular analysis of PCa samples is affected by this heterogeneity, which often masks the aggressive signature of truly metastatic cells. As a consequence, the development of gene expression profile-based diagnostic and prognostic algorithms is particularly challenging in PCa.

As described herein, the transcriptome of tumor tissue cell lines derived from two PCa biopsies of the same patient were analyzed. When engrafted in the sub-enal capsule of immunocompromised mice, one tumor tissue line invariably gave rise to localized and poorly-invasive tumors; the other line was reproducibly able to generate highly invasive tumors, producing distant metastases through predictable routes. Since the two tumor tissue lines are derived from the same patient and share most of the genetic alterations with the donor tissue, they represent an ideal model to study gene expression changes related to PCa progression to a metastatic state. A similar model had been successfully exploited for the identification of PCa-associated miRNAs and protein coding genes (10, 14).

Data from 4 independent datasets and more than 600 human samples confirmed that this gene is prostate-specific and highly up-regulated in PCa. Even though the PCAT18 transcript has been reported before (Ota T, et al. Complete sequencing and characterization of 21.243 full-length human cDNAs. Nature genetics. 2004; 36(1):40-45), its function and expression profile and utility has not been previously determined. Two previous studies performed whole-genome lncRNA expression profiling in PCa. However, due to the highly heterogeneous nature of lncRNAs, and to the fact that most lncRNAs have not been characterized, the available analysis tools do not cover all the lncRNAs expressed by a cell. Chinnaiyan and co-workers previously published a list of 121 unannotated lncRNAs expressed in PCa (19). Since they deliberately filtered out transcripts present in the RefSeq database they likely excluded PCAT18 from their analysis. More recently. Ren and co-workers published a list of lncRNAs expressed in PCa based on the fRNAdb database (20). All the sequences matching PCAT18 were actively searched for in this database, and it was found that this locus is covered by just 4 short sequences that span <10% of the entire transcript (www.genecode.com). Moreover, PCAT18-matching sequences were not found in Ren's list of PCa-associated lncRNAs Since Ren et al. only analyzed sequences longer than 200 bp, it was assumed they filtered out the short PCAT18-matching sequences present in fRNAdb database.

The data herein indicates that PCAT18 is more over-expressed in PCa than PCGEM1 and that a set of patients over-expressing this gene does not express PCA3 (cBio portal, data not shown). Since PCAT18 is so frequently over-expressed in PCa cells and PCa-specific, its measurement in plasma samples (alone or in combination with other non-coding RNAs) can allow earlier and more accurate detection of PCa progression to a metastatic and drug-resistant stage. lncRNA is detectable in plasma samples from PCa patients and can discriminate between localized and mCRPC.

### REFERENCES

1. Kirby M. Hirst C, Crawford E D. Characterising the castration-resistant prostate cancer population: a systematic review. International journal of clinical practice. 2011; 65:1180-92.
2. Rodrigues G, Warde P, Pickles T, Crook J, Brundage M, Souhami L, et al. Pre-treatment risk stratification of prostate cancer patients: A critical review. Canadian Urological Association journal=Journal de l'Association des urologues du Canada. 2012; 6:121-7.
3. Kapranov P. Cheng J, Dike S, Nix D A, Duttagupta R, Willingham A T, et al. RNA maps reveal new RNA classes and a possible function for pervasive transcription. Science 2007; 316:1484-8.
4. Watahiki A, Macfarlane R J, Gleave M E, Crea F, Wang Y, Helgason C D, et al. Plasma miRNAs as Biomarkers to Identify Patients with Castration-Resistant Metastatic Prostate Cancer. International journal of molecular sciences. 2013; 14:7757-70.
5. Chen G. Wang Z, Wang D, Qiu C, Liu M, Chen X, et al. LncRNADisease: a database for long-non-coding RNA-associated diseases. Nucleic acids research. 2013; 41:D983-6.
6. Gibb E A, Brown C J, Lam W L. The functional role of long non-coding RNA in human carcinomas. Molecular cancer. 2011; 10:38.
7. Bussemakers M J, van Bokhoven A, Verhaegh G W, Smit F P, Karthaus H F, Schalken J A, et al. DD3: a new prostate-specific gene, highly overexpressed in prostate cancer. Cancer research. 1999; 59:5975-9.
8. Srikantan V, Zou Z, Petrovics G, Xu L, Augustus M, Davis L, et al. PCGEM1, a prostate-specific gene, is overexpressed in prostate cancer. Proceedings of the National Academy of Sciences of the United States of America. 2000; 97:12216-21.
9. Leyton G H, Hessels D, Jannink S A, Smit F P, de Jong H, Cornel E B, et al. Prospective Multicentre Evaluation of PCA3 and TMPRSS2-ERG Gene Fusions as Diagnostic and Prognostic Urinary Biomarkers for Prostate Cancer. European urology. 2012.
10. Watahiki A, Wang Y, Morris J, Dennis K, O'Dwyer H M, Gleave M, et al. MicroRNAs associated with metastatic prostate cancer. PloS one. 2011; 6:e24950.
11. Mortazavi A, Williams B A, McCue K, Schaeffer L, Wold B. Mapping and quantifying mammalian transcriptomes by RNA-Seq. Nature methods. 2008; 5:621-8.
12. Cerami E. Gao J, Dogrusoz U, Gross B E, Sumer S O, Aksoy B A, et al. The cBio cancer genomics portal: an open platform for exploring multidimensional cancer genomics data. Cancer discovery. 2012; 2:401-4.
13. Taylor B S, Schultz N, Hieronymus H. Gopalan A, Xiao Y, Carver B S, et al. Integrative genomic profiling of human prostate cancer. Cancer cell. 2010; 18:11-22.
14. Lin D, Watahiki A, Bayani J, Zhang F, Liu L, Ling V, et al. ASAP1, a gene at 8q24, is associated with prostate cancer metastasis. Cancer research. 2008; 68:4352-9.
15. Luo M, Li Z, Wang W, Zeng Y, Liu Z. Qiu J. Long non-coding RNA H19 increases bladder cancer metastasis by associating with EZH2 and inhibiting E-cadherin expression. Cancer letters. 2013.
16. Poliseno L, Salmena L, Zhang J, Carver B, Haveman W J, Pandolfi P P. A coding-independent function of gene and pseudogene mRNAs regulates tumour biology. Nature. 2010; 465:1033-8
17. Fickett J W. Recognition of protein coding regions in DNA sequences. Nucleic acids research. 1982; 10:5303-18.
18. Lin D, Bayani J, Wang Y, Sadar M D, Yoshimoto M, Gout P W, et al. Development of metastatic and non-metastatic tumor lines from a patients prostate cancer specimen-identification of a small subpopulation with metastatic potential in the primary tumor. The Prostate. 2010; 70:1636-44.
19. Prensner J R, Iyer M K, Balbin O A, Dhanasekaran S M, Cao Q, Brenner J C, et al. Transcriptome sequencing across a prostate cancer cohort identifies PCAT-1, an unannotated lincRNA implicated in disease progression. Nature biotechnology. 2011; 29:742-9.
20. Ren S, Peng Z, Mao J H, Yu Y, Yin C, Gao X, et al. RNA-seq analysis of prostate cancer in the Chinese population identifies recurrent gene fusions, cancer-associated long noncoding RNAs and aberrant alternative splicings. Cell research. 2012; 22:806-21.

1. A method for diagnosing prostate cancer in a subject suspected of having prostate cancer comprising:
(a) assessing the expression level of PCAT18 in a biological sample obtained from the subject;
(b) comparing the expression level of PCAT18 in the biological sample to a reference expression level; and
(c) identifying the subject as having prostate cancer when the expression level of PCAT18 in the biological sample is greater than the reference expression level, or identifying the subject as not having prostate cancer when the expression level of PCAT18 in the biological sample is not greater than the reference expression level.

2. The method of claim 1, wherein the expression level of PCAT18 is assessed by evaluating the amount of PCAT18 RNA in the biological sample.

3. The method of claim 2, wherein the PCAT18 RNA comprises the nucleotide sequence corresponding to SEQ ID NO:1.

4. The method of claim 1, wherein the method for diagnosing prostate cancer is used in combination with an assessment of one or more than one additional prostate cancer biomarker.

5. The method of claim 4, wherein the one or more than one additional prostate cancer biomarker is PSA, PCGEM1, PCA3, or a combination thereof.

6. (canceled)

7. A method for monitoring a treatment for prostate cancer in a subject diagnosed with prostate cancer comprising:
(a) obtaining a baseline level by assessing the expression level of PCAT18 in a biological sample obtained from the subject prior to administration of the treatment;
(b) administering the treatment to the subject for a treatment period;
(c) after the treatment period, assessing the expression level of PCAT18 in a second biological sample obtained from the subject;
(d) comparing the expression level of PCAT18 in the second biological sample to the baseline level; and
(e) identifying a poor response to the treatment when the expression level of PCAT18 in the second biological sample is greater than the baseline level, or identifying a good response to the treatment when the expression level of PCAT18 in the second biological sample is not greater than the baseline level.

8. A method of treating a subject diagnosed with prostate cancer by administering a therapeutically effective amount of the pharmaceutical composition of claim 11.

9. The method of claim 8, wherein the siRNA comprises an antisense nucleotide sequence corresponding to SEQ ID NO:22 or SEQ ID NO:23.

10. The method of claim 8, wherein the antisense oligonucleotide comprises a nucleotide sequence corresponding to SEQ ID NO:24 or SEQ ID NO:25.

11. A pharmaceutical composition comprising a therapeutic agent effective to reduce an amount of PCAT18 in cancerous prostate cells exposed to the therapeutic agent, and a pharmaceutically acceptable carrier, wherein the therapeutic agent is an antisense oligonucleotide, an siRNA, or a combination thereof.

12. The pharmaceutical composition of claim 11, wherein the siRNA comprises an antisense nucleotide sequence corresponding to SEQ ID NO:22 or SEQ ID NO:23.

13. The pharmaceutical composition of claim 11, wherein the antisense oligonucleotide comprises a nucleotide sequence corresponding to SEQ ID NO:24 or SEQ ID NO:25.

14-17. (canceled)


SEQUENCE LISTING (26 sequences)

SEQ ID NO: 1 - DNA, 2598 bp, Homo sapiens
ctttttcttcgtaacttcatggcaacatgacctacagttactcatttttcaggcctgagttgatcgttaatcatcttaattatgttcattctgaagccaacaggagaaccaagaccaaaactttattgtctctgctttcatttcttgatgaaacctctggactaagcacacatcttccttgtttatctctctcaaaggagtgtggagtgcttcatctggacatccacgggaagaaggaagacatgagggaatgctggaagaggagacaggccccagatttgggcaggaagtaaacagttttcaggctgaggccaatctgagcaggaacattccaatatttcttcagctacgttgtcccagcacttcactggttaaccttttatgtccaccatttgtggatttcacagctacttgtcaatggtgaatattgatcatcatcattatctactgagctgctaccatatcccagctactccttgcatgttgttcattattttctcaacactcagcatatttgcaatatgttatgtaatatcacagacaaggaaactgaacgcagaaatgttttatttcttgccaaacatcacatgaggatgaacaatgaaaccgatttgaaaccaggattgtctgattccaacatctctgggtcctttttcactctgatatgctgcaattaaaaagccatttctaagactgtacgttccaagattgtttctatagttggttgtgagagaagtttctctgaacttgcagagcacgggaaaccaggaggaggaggctcagggtgctctccacacgtgtgaaaccggctgtgggtgttgctttcctgtgactgccatttgccactgatgatcttctcttcctctggaagagtaagagggggaaggacacagtttcagtgcttcccatttataattaattaattaattaagggcaggtgcaggggctcacacttgtaaccccagcactttaggagattgagcaaggaggatcccctgagcctgggagtttgagatcagcctgggaagcagagtgagaccatgacttaaataaaaatttttttgtgttttgttctaatttttacttttttgagacagggtctcgctctgttgcccaggctggagtgcagtagcgtgatctcagctcactgcaacctctgcctctgggttcaaacaattctcatgcctcagcctcccaagtagctgggattacaggtgcctgccaccatgcccaactaatttttttgtacttttagtagagacgggttttcaccatgttggttggccaggctggtctcgaactcctgacctcaagtgatccgcccacctcagcctcccaaagtgctgggattacaggcatgagccaccacgcccgaccaaaaaaaaatttttttttttcaattagccagacattgtggcatgtgcctgtagtcgcagctaccatgggggctgaggtgggaggatagcttgagcccaggagtttgaaaccagcctgggcagcaaagtgagaccctgcctcaaaaataaaaaataaaaaaaaagctatttcttacagatattcattgagtggaaagcaatttaattttacgtataataagttatttcaaaaagaattgtagttatgtgtggcaatttttcataaatacctgatacaaatcaagatctaccatggtaagtgtgctccagagatacccttcttgaccaaaacaaagcagggcagggattgggaggccttttgggctgggctccaggacacaatgactagaagatgatgcaaagtgcatgacagagtagagagctacacagtttacacatattagactctgcacatattagagtctacacatattagactctgcaaaagtagttcttaaatgttagctcacatgcaccccagtcacgttactgagcagattcctgggctccagtcctgggtgattcttgcaggtggtccattgaccagattaggagaatcatcatccaaggtctcaaagccaaatttaggacgcagagagaaagaatcctaagtggttgaatgggaaactgattctcctcttgagcacaccttagattcaaataattgattggagaagatcattccatacaaaaatcttctgacctgcagagtttctacaaaaagtcttgagaaagaaaaagaaaaacatatatttttggaaaagattgaatttaattataattttgcaaattatactgtgggaaaggtcaacaaaaacaagcgtattgcggatgatcttataaaaagaaccttttgtaaagtaaacaattaccttctgatttgtctactctgaacccttgatgtttatttaaattggatgttctcatagcaggacacacctgatacctggcttgtctaaacacttagaaagtcaaattgtctagttatcaaagcccaggcttattttctaaataagtatggatggtctttcaggagtcatttcacagtgatgtgcagagagaaaaacaatacaaacaaacaaacaaacaaacaaaaaacaaatcattgttcctaagtgtgttttccagcctatgtctaactttctagcagataatggcaataaaaatgtttgggtccaacatt

SEQ ID NO: 2 - DNA, 22 bp, Artificial sequence
  Forward Primer - HPRT1
ggtcaggcagtataatccaaag

SEQ ID NO: 3 - DNA, 23 bp, Artificial sequence
  Reverse Primer - HPRT1
cgatgtcaataggactccagatg

SEQ ID NO: 4 - DNA, 21 bp, Artificial sequence
  Forward Primer GAPDH
caccagggctgcttttaactc

SEQ ID NO: 5 - DNA, 20 bp, Artificial sequence
  Reverse primer - GAPDH
gacaagcttcccgttctcag

SEQ ID NO: 6 - DNA, 20 bp, Artificial sequence
  Forward primer - PCAT18
aggagacaggccccagattt

SEQ ID NO: 7 - DNA, 20 bp, Artificial sequence
  Reverse primer - PCAT18
tgaagtgctgggacaacgta

SEQ ID NO: 8 - DNA, 20 bp, Artificial sequence
  Forward primer - PCGEM1
ttgccctatgccgtaacctg

SEQ ID NO: 9 - DNA, 20 bp, Artificial sequence
  Reverse primer - PCGEM1
acgttgagtcccagtgcatc

SEQ ID NO: 10 - DNA, 21 bp, Artificial sequence
  Forward primer - H19
ccagtgaggagtgtggagtag

SEQ ID NO: 11 - DNA, 20 bp, Artificial sequence
  Reverse primer - H19
cagctgccacgtcctgtaac

SEQ ID NO: 12 - DNA, 20 bp, Artificial sequence
  Forward primer - Linc461_1
aggaaacagctctggcatcc

SEQ ID NO: 13 - DNA, 20 bp, Artificial sequence
  Reverse primer - Linc461_1
cagattccccaccccctttc

SEQ ID NO: 14 - DNA, 20 bp, Artificial sequence
  Forward primer - Linc461_3
gttcctgcccagctggattt

SEQ ID NO: 15 - DNA, 20 bp, Artificial sequence
  Reverse primer - Linc461_3
tcagagtagtccacgccaga

SEQ ID NO: 16 - DNA, 22 bp, Artificial sequence
  Forward primer - LOC285419
tgactcaacttctggtgcagat

SEQ ID NO: 17 - DNA, 24 bp, Artificial sequence
  Reverse primer - LOC285419
ggatgtggcatatctcttggttta

SEQ ID NO: 18 - DNA, 22 bp, Artificial sequence
  Forward primer - NCRNA116
gagactgctcagaggaagagaa

SEQ ID NO: 19 - DNA, 20 bp, Artificial sequence
  Reverse primer - NCRNA116
cagacagcccagtgtcttgg

SEQ ID NO: 20 - DNA, 21 bp, Artificial sequence
  Forward primer - KLK3 (PSA)
agtgcgagaagcattcccaac

SEQ ID NO: 21 - DNA, 22 bp, Artificial sequence
  Reverse primer - KLK3 (PSA)
ccagcaagatcacgcttttgtt

SEQ ID NO: 22 - RNA, 36 nt, Artificial sequence
  siRNA1
agcaggaacauuccaauagaagaaauauuggaaugu

SEQ ID NO: 23 - RNA, 36 nt, Artificial sequence
  siRNA2
gcaacaugaccuacaguuaaugaguaacuguagguc

SEQ ID NO: 24 - DNA, 20 bp, Artificial sequence
  Antisense oligonucleotide ASO2
tcctgctcagattggcctca

SEQ ID NO: 25 - DNA, 20 bp, Artificial sequence
  Antisense oligonucleotide ASO7
acgcgcgactatacgcgcct

SEQ ID NO: 26 - DNA, 20 bp, Artificial sequence
  Antisense oligonucleotide NC
caacacccacagccggtttc